SEQUENCE LISTING (72 sequences)

SEQ ID NO: 1 - DNA, 22 bp, synthetic construct
  source 1..22: mol_type = other DNA
gcacaatggcacggttgaatgt

SEQ ID NO: 2 - DNA, 22 bp, synthetic construct
  source 1..22: mol_type = other DNA
aaaggagtgaacagggtgtggg

SEQ ID NO: 3 - DNA, 22 bp, synthetic construct
  source 1..22: mol_type = other DNA
tggtcagacaggaaaccagtgg

SEQ ID NO: 4 - DNA, 22 bp, synthetic construct
  source 1..22: mol_type = other DNA
gggaggcgggtagtaagaatgc

SEQ ID NO: 5 - DNA, 19 bp, synthetic construct
  source 1..19: mol_type = other DNA
caaggtgtacagtgacgca

SEQ ID NO: 6 - DNA, 21 bp, synthetic construct
  source 1..21: mol_type = other DNA
aaggagagaacctctagctcc

SEQ ID NO: 7 - DNA, 22 bp, synthetic construct
  source 1..22: mol_type = other DNA
atgttgacaatgcgtctctggc

SEQ ID NO: 8 - DNA, 22 bp, synthetic construct
  source 1..22: mol_type = other DNA
ctgttcctgaatctgagcctgc

SEQ ID NO: 9 - DNA, 22 bp, synthetic construct
  source 1..22: mol_type = other DNA
gtcagcatgcggtatgaggaag

SEQ ID NO: 10 - DNA, 22 bp, synthetic construct
  source 1..22: mol_type = other DNA
ccagttgtcttcttcccagtcc

SEQ ID NO: 11 - DNA, 20 bp, synthetic construct
  source 1..20: mol_type = other DNA
tgggaagccgtctgtggaag

SEQ ID NO: 12 - DNA, 26 bp, synthetic construct
  source 1..26: mol_type = other DNA
cctacgtttctcaatgactgcagtct

SEQ ID NO: 13 - DNA, 21 bp, synthetic construct
  source 1..21: mol_type = other DNA
cgtcacctcaagtgccacaac

SEQ ID NO: 14 - DNA, 22 bp, synthetic construct
  source 1..22: mol_type = other DNA
ttcttcaggtgggactccagag

SEQ ID NO: 15 - DNA, 22 bp, synthetic construct
  source 1..22: mol_type = other DNA
ccattctcccatcccaggaagg

SEQ ID NO: 16 - DNA, 22 bp, synthetic construct
  source 1..22: mol_type = other DNA
tgctggaggtttcagagatggg

SEQ ID NO: 17 - DNA, 29 bp, synthetic construct
  source 1..29: mol_type = other DNA
gaagtagctgaggcatacgaggtattatc

SEQ ID NO: 18 - DNA, 23 bp, synthetic construct
  source 1..23: mol_type = other DNA
tggaatgtgaagccgtactcaca

SEQ ID NO: 19 - DNA, 22 bp, synthetic construct
  source 1..22: mol_type = other DNA
tcagggtagaaaaggcacaggg

SEQ ID NO: 20 - DNA, 22 bp, synthetic construct
  source 1..22: mol_type = other DNA
cctcttgaacttttgcgagggc

SEQ ID NO: 21 - DNA, 20 bp, synthetic construct
  source 1..20: mol_type = other DNA
aagggtcatcatctctgccc

SEQ ID NO: 22 - DNA, 20 bp, synthetic construct
  source 1..20: mol_type = other DNA
catggactgtggtcatgagt

SEQ ID NO: 23 - DNA, 16 bp, synthetic construct
  source 1..16: mol_type = other DNA
gccgccagctcaccat

SEQ ID NO: 24 - DNA, 20 bp, synthetic construct
  source 1..20: mol_type = other DNA
tcgtcgcccacataggaatc

SEQ ID NO: 25 - DNA, 24 bp, synthetic construct
  source 1..24: mol_type = other DNA
ggtaagactcggaccaaggacaag

SEQ ID NO: 26 - DNA, 22 bp, synthetic construct
  source 1..22: mol_type = other DNA
tccgccggattgtgatgtaacg

SEQ ID NO: 27 - DNA, 23 bp, synthetic construct
  source 1..23: mol_type = other DNA
gttaacgtgacgggagatgaggt

SEQ ID NO: 28 - DNA, 21 bp, synthetic construct
  source 1..21: mol_type = other DNA
cttctgagaccaggacgcagg

SEQ ID NO: 29 - DNA, 22 bp, synthetic construct
  source 1..22: mol_type = other DNA
tgaaggaaccgctagaagcaca

SEQ ID NO: 30 - DNA, 22 bp, synthetic construct
  source 1..22: mol_type = other DNA
cacaggcaacccaggctaaatg

SEQ ID NO: 31 - DNA, 22 bp, synthetic construct
  source 1..22: mol_type = other DNA
tcagggcgagatcaccatcttg

SEQ ID NO: 32 - DNA, 23 bp, synthetic construct
  source 1..23: mol_type = other DNA
gacaccattgaacttgtccgctg

SEQ ID NO: 33 - DNA, 22 bp, synthetic construct
  source 1..22: mol_type = other DNA
agacctcacctgcatggacttc

SEQ ID NO: 34 - DNA, 22 bp, synthetic construct
  source 1..22: mol_type = other DNA
agctcgatgaagtggttgctga

SEQ ID NO: 35 - DNA, 22 bp, synthetic construct
  source 1..22: mol_type = other DNA
ggagactgggagaaaactcgct

SEQ ID NO: 36 - DNA, 22 bp, synthetic construct
  source 1..22: mol_type = other DNA
ggtgacacatgaaagctggtcg

SEQ ID NO: 37 - DNA, 22 bp, synthetic construct
  source 1..22: mol_type = other DNA
cagggtagtgaacagacagcca

SEQ ID NO: 38 - DNA, 22 bp, synthetic construct
  source 1..22: mol_type = other DNA
aagtcgtctactccctccagga

SEQ ID NO: 39 - DNA, 22 bp, synthetic construct
  source 1..22: mol_type = other DNA
ctcacaggacccaaacatccct

SEQ ID NO: 40 - DNA, 22 bp, synthetic construct
  source 1..22: mol_type = other DNA
caagaacctcacttcgctgctg

SEQ ID NO: 41 - DNA, 22 bp, synthetic construct
  source 1..22: mol_type = other DNA
taaccgtggagtctgaggagga

SEQ ID NO: 42 - DNA, 22 bp, synthetic construct
  source 1..22: mol_type = other DNA
ccctcttggagaacctcactgg

SEQ ID NO: 43 - DNA, 22 bp, synthetic construct
  source 1..22: mol_type = other DNA
gcttcggagatggagtcccttt

SEQ ID NO: 44 - DNA, 22 bp, synthetic construct
  source 1..22: mol_type = other DNA
gaccaagcccctaggagacttg

SEQ ID NO: 45 - DNA, 25 bp, synthetic construct
  source 1..25: mol_type = other DNA
cagtgacaaagacactgaccagagg

SEQ ID NO: 46 - DNA, 23 bp, synthetic construct
  source 1..23: mol_type = other DNA
tcctccacggacactatctcagg

SEQ ID NO: 47 - DNA, 22 bp, synthetic construct
  source 1..22: mol_type = other DNA
atcccgactgtaaatcctccgc

SEQ ID NO: 48 - DNA, 22 bp, synthetic construct
  source 1..22: mol_type = other DNA
gaggctgggagacgagttttga

SEQ ID NO: 49 - DNA, 24 bp, synthetic construct
  source 1..24: mol_type = other DNA
ggaagagagcaagttcaagcaagc

SEQ ID NO: 50 - DNA, 23 bp, synthetic construct
  source 1..23: mol_type = other DNA
actctctccacttcatctgcagc

SEQ ID NO: 51 - DNA, 22 bp, synthetic construct
  source 1..22: mol_type = other DNA
caggtccataaagctcgggtga

SEQ ID NO: 52 - DNA, 22 bp, synthetic construct
  source 1..22: mol_type = other DNA
tgccttaggtccagctgtacac

SEQ ID NO: 53 - DNA, 22 bp, synthetic construct
  source 1..22: mol_type = other DNA
tagcgatggagtccttgttgca

SEQ ID NO: 54 - DNA, 22 bp, synthetic construct
  source 1..22: mol_type = other DNA
tgttcagatgaccccagttgct

SEQ ID NO: 55 - DNA, 22 bp, synthetic construct
  source 1..22: mol_type = other DNA
gtctccaggctccgtgttctag

SEQ ID NO: 56 - DNA, 22 bp, synthetic construct
  source 1..22: mol_type = other DNA
cccacagtctccccgagatttt

SEQ ID NO: 57 - DNA, 21 bp, synthetic construct
  source 1..21: mol_type = other DNA
ggcatgcaagcagcttagagg

SEQ ID NO: 58 - DNA, 22 bp, synthetic construct
  source 1..22: mol_type = other DNA
accaaggggaggaaccattctg

SEQ ID NO: 59 - DNA, 22 bp, synthetic construct
  source 1..22: mol_type = other DNA
caagggtgtgagaacatgtgcc

SEQ ID NO: 60 - DNA, 22 bp, synthetic construct
  source 1..22: mol_type = other DNA
tgagtcaaaacaagcaagggcc

SEQ ID NO: 61 - DNA, 22 bp, synthetic construct
  source 1..22: mol_type = other DNA
atggcatgaggaggagcaagag

SEQ ID NO: 62 - DNA, 22 bp, synthetic construct
  source 1..22: mol_type = other DNA
tcatcgccccgtttcctacttt

SEQ ID NO: 63 - DNA, 22 bp, synthetic construct
  source 1..22: mol_type = other DNA
cacatccaccaaactcctgcct

SEQ ID NO: 64 - DNA, 24 bp, synthetic construct
  source 1..24: mol_type = other DNA
tattgacaggcggcattgttcctt

SEQ ID NO: 65 - DNA, 22 bp, synthetic construct
  source 1..22: mol_type = other DNA
gctgttgctgtgccatcgaata

SEQ ID NO: 66 - DNA, 22 bp, synthetic construct
  source 1..22: mol_type = other DNA
gttgtcatctaggagccgctga

SEQ ID NO: 67 - DNA, 23 bp, synthetic construct
  source 1..23: mol_type = other DNA
gccctttgcattgttcgtaccac

SEQ ID NO: 68 - DNA, 22 bp, synthetic construct
  source 1..22: mol_type = other DNA
aagaaagtggaatcgcctccca

SEQ ID NO: 69 - DNA, 22 bp, synthetic construct
  source 1..22: mol_type = other DNA
gcatctgtggctcttgtgcaat

SEQ ID NO: 70 - DNA, 22 bp, synthetic construct
  source 1..22: mol_type = other DNA
atactgctgcttgccttcctga

SEQ ID NO: 71 - DNA, 22 bp, synthetic construct
  source 1..22: mol_type = other DNA
tgccagggaagactacaaggtg

SEQ ID NO: 72 - DNA, 22 bp, synthetic construct
  source 1..22: mol_type = other DNA
agtgctcctcaaagggcttctt

(19) United States
(12) Patent Application Publication
(10) Pub. No.: US 2026/0317703 A1
(43) Pub. Date: Oct. 15, 2026

(54) METHOD FOR IN VITRO GENERATION OF HUMAN SPERMATIDS

(71) Applicant: PATERNA BIOSCIENCES INC., SALT LAKE CITY, UT (US)

(72) Inventors: Xichen Nie, Salt Lake City, UT (US); Qianhui Du, Salt Lake City, UT (US); Alek Dylan Peterlin, Millcreek, UT (US); David Aaron Kircher, Salt Lake City, UT (US); Graham McGuire Hickey, Saratoga Springs, UT (US); Bingqian Guo, Millcreek, UT (US); Igal Steven Sterin, Salt Lake City, UT (US); James Morris Hoteling, Salt Lake City, UT (US); Bradley Robert Cairns, Salt Lake City, UT (US); Gabriela Ligia Cosma, Philadelphia, PA (US); Joseph Raymond Casalini, Salt Lake City, UT (US); Alexander Wojciech Pastuszak, Salt Lake City, UT (US)

(21) Appl. No.: 19/670,886

(22) Filed: May 7, 2026

Related U.S. Application Data
(63) Continuation of application No. 19/257,274, filed on Jul. 1, 2025.
(60) Provisional application No. 63/666,348, filed on Jul. 1, 2024.

(51) Int. Cl. C12N 5/076

(52) U.S. Cl. CPC C12N 5/061; C12N 2500/32; C12N 2500/44; C12N 2500/84; C12N 2501/125; C12N 2501/155; C12N 2501/16; C12N 2501/31; C12N 2501/33; C12N 2501/392; C12N 2501/415; C12N 2501/999; C12N 2502/02; C12N 2506/04

(57) ABSTRACT

A sequential cell culture system and methods for the in vitro generation of human spermatozoa from differentiating spermatogonia (dSPGs) and spermatogonial stem cells (SSCs) is provided. The instant disclosure encompasses the discovery and optimization of distinct, stage-specific culture media and conditions that enable, for the first time, the complete progression of human spermatogenesis outside the body. The process includes: (1) inducing entry of dSPGs and SSCs into meiosis; (2) supporting meiotic progression and completion to round spermatids; and (3) promoting spermiogenesis and the formation of elongated, spermatozoa. The invention further provides compositions, systems, and protocols for each stage, as well as markers and methods for identifying successful progression through each developmental transition. This system enables, for the first time, the complete in vitro recapitulation of human spermatogenesis, providing a platform for research, infertility treatment, and assisted reproductive technologies.

### CROSS-REFERENCE TO RELATED APPLICATIONS

This application is a continuation of U.S. application Ser. No. 19/257,274 filed on Jul. 1, 2025, titled “METHOD FOR IN VITRO GENERATION OF HUMAN SPERMATIDS,” which claims the benefit of U.S. Provisional Application No. 63/666,348, filed Jul. 1, 2024, both of which is incorporated by reference herein in its entirety.

### FIELD OF THE INVENTION

The present disclosure provides cultures and culture methods for in vitro generation of human spermatids.

### INCORPORATION OF SEQUENCE LISTING

The present application contains a Sequence Listing that has been submitted in .XML format via PatentCenter and is hereby incorporated herein by reference in its entirety. Said .XMl was created on Jul. 1, 2025, is named PCT3_Sequence_Listing.xml, and is 64,585 bytes in size.

### FIELD OF THE INVENTION

The present disclosure relates to cell culture systems and methods for the in vitro generation of human spermatogenic cells, including the sequential differentiation of human spermatogonial stem cells and differentiating spermatogonia to produce spermatozoa.

### BACKGROUND OF THE INVENTION

The field of reproductive biology and regenerative medicine has long sought to understand and replicate the complex process of spermatogenesis, the development of mature spermatozoa from undifferentiated germ cells. Spermatogenesis is a highly orchestrated, multi-stage process involving the proliferation and differentiation of spermatogonial stem cells, entry into and progression through meiosis, and the final transformation of haploid round spermatids into elongated, flagellated spermatozoa. This process is essential for male fertility and the transmission of genetic information to the next generation.

While significant advances have been made in elucidating the molecular and cellular mechanisms underlying spermatogenesis in model organisms such as mice, the ability to recapitulate the full process of human spermatogenesis in vitro has remained elusive. In particular, the successful induction of meiotic entry, progression through the complex stages of meiosis, and the subsequent differentiation of human germ cells into functional spermatids and spermatozoa have not been achieved in a controlled culture environment. Previous attempts have been limited to inefficient and partial differentiation or have failed to support the complete sequence of developmental transitions required for the generation of mature human sperm cells.

The inability to achieve human spermatogenesis in vitro has posed a major barrier to both basic research and clinical applications, including the study of male infertility, the development of new contraceptive strategies, and the creation of novel assisted reproductive technologies. The lack of robust, reproducible culture systems has also hindered efforts to investigate the genetic and epigenetic regulation of human germ cell development, as well as the impact of environmental and pharmacological factors on male reproductive health.

Accordingly, there is a need for cell culture systems that enables the in vitro recapitulation of spermatogenesis of human spermatogonia to spermatozoa.

### SUMMARY OF THE INVENTION

One aspect of the instant disclosure encompasses a method of generating human spermatozoa from differentiating spermatogonia (dSPGs) or spermatogonial stem cells (SSCs) in vitro. The method comprises culturing human dSPGs or SSCs in a first culture medium of a first culture system; transferring the cells resulting from culturing in the first culture medium to a second culture medium of a second culture system and culturing the cells using the second culture system; followed by transferring the cells resulting from culturing in the second culture system to a third culture medium of a third culture system and culturing the cells in the third culture system to obtain spermatozoa.

The first culture medium comprises a nutrient-rich basal medium and a serum or serum substitute, wherein the nutrient-rich basal medium is present at a sufficiently low concentration to provide a nutrient-restricted medium, wherein the sufficiently low concentration of nutrient-rich basal medium ranges from about 8% to about 12% of the total medium volume and the balance comprising a diluent or buffered salt solution at a concentration ranging from about 88% to about 92%; retinoic acid at a concentration ranging from about 4 uM to about 6 uM; BMP2, BMP4 and BMP7 each at a concentration ranging from about 15 ng/ml to about 25 ng/ML; activin A at a concentration ranging from about 90 ng/ml to about 110 ng/mL; insulin at a concentration ranging from about 40 ug/mL to about 60 ug/mL; and IWR-endo-1 at a concentration ranging from about 2 uM to about 4 uM.

The dSPGs and SSCs are cultured using the first culture medium at a temperature ranging from about 31° C. to about 33° C. for a first culture period sufficient for entry of the human dSPGs or SSCs into meiosis. In some aspects, the first culture period is about 1 day to about 4 days.

Entry of the human dSPGs and SSCs into meiosis is evidenced by EdU incorporation indicating DNA replication and the appearance of spermatocytes arrested before the pachytene stage of meiosis I as characterized by the presence, absence, or localization of HORMAD1, γH2AX, DMC1, RAD51, STRA8, or any combination thereof. In some aspects, entry of the human dSPGs and SSCs into meiosis can be further evidenced by the appearance of spermatocytes arrested before the pachytene stage of meiosis I characterized by EdU incorporation indicating DNA replication; the presence of HORMAD1 on unsynapsed chromosome axes and presence of γH2AX diffusely throughout the nucleus, prior to its restriction to the sex body; absence, reduction, or relocalization of HORMAD1 and γH2AX from autosomes; or any combination thereof.

The second culture system comprises a second culture medium and MEF feeder cells. The second culture medium comprises a nutrient-rich basal medium and a serum or serum substitute, wherein the nutrient-rich basal medium is present at a sufficiently high concentration to provide a nutrient-rich medium, wherein the sufficiently high concentration of nutrient-rich basal medium ranges from about 85% to about 95% of the total medium volume; FSH at a concentration ranging from about 15 ng/ml to about 25 ng/mL; testosterone at a concentration ranging from about 9 uM to about 11 uM; bovine pituitary extract (BPE) at a concentration ranging from about 40 ug/mL to about 60 ug/mL; L-Glutamine or a L-alanyl-L-glutamine dipeptide at a concentration ranging from about 0.5 mM to about 5 mM; and optionally c-KIT receptor ligand (KITLG) at a concentration ranging from about 0.5 ng/ml to about 1.5 ng/mL.

The cells resulting from culturing in the first culture medium are cultured at a temperature ranging from about 31° C. to about 33° C. for a second culture period sufficient for the progression of the human spermatocytes arrested before the pachytene stage of meiotic prophase I through completion of meiosis and formation of round spermatids. In some aspects, the second culture period ranges from about 8 days to about 12 days.

Progression of the human spermatocytes arrested before the pachytene stage of meiotic prophase I through completion of meiosis and formation of round spermatids is evidenced b by the appearance of cells expressing ACRV1.

The third culture medium comprises a nutrient-rich basal medium and a serum or serum substitute, wherein the nutrient-rich basal medium is present at a sufficiently high concentration to provide a nutrient-rich medium, wherein the sufficiently high concentration of nutrient-rich basal medium ranges from about 85% to about 95% of the total medium volume; 2-mercaptoethanol at a concentration ranging from about 45 uM to about 55 uM; testosterone at a concentration ranging from about 3 uM to about 7 uM; retinoic acid at a concentration ranging from about 4 uM to about 6 uM; and conditioned media comprising MEF-conditioned third culture medium, lactate at a concentration ranging from about 0.8 mM to about 1.2 mM, or a combination thereof. In some aspects, the third culture medium comprises MEF-conditioned third culture medium at a rate of one part MEF-conditioned third cell culture medium to three parts third culture medium.

The third culture system is used to culture the cells in the third culture system at a temperature ranging from about 31° C. to about 33° C. for a third culture period sufficient for generation of human spermatozoa from the round spermatids. Generation of human spermatozoa from the round spermatids is evidenced by the appearance of flagellar cells. In some aspects, the third culture period is about 5 to about 8 days.

In some aspects, the first culture medium comprises αMEM basal medium at a concentration ranging from about 8% to about 12%. In some aspects, the first culture medium comprises KSR serum substitute at a concentration ranging from about 0.1% to about 2%. In some aspects, the first culture medium comprises αMEM basal medium at a concentration ranging from about 8% to about 12%, EBSS at a concentration ranging from about 88% to about 92%, KSR serum substitute at a concentration ranging from about 0.1% to about 2%, BMP2, BMP4, and BMP7 each at a concentration ranging from about 15 ng/ml to about 25 ng/ml, retinoic acid at a concentration ranging from about 4 uM to about 6 uM, activin A at a concentration ranging from about 90 ng/ml to about 110 ng/ml, insulin at a concentration ranging from about 40 ug/mL to about 60 ug/mL, and IWR-endo-1 at a concentration ranging from about 2 uM to about 4 uM.

### BRIEF DESCRIPTION OF THE FIGURES

The following drawings form part of the present specification and are included to further demonstrate certain embodiments of the present disclosure. Certain embodiments can be better understood by reference to one or more of these drawings in combination with the detailed description of specific embodiments presented herein.

FIG. 1 diagrammatically depicts the stages during differentiation of adult spermatogonial stem cells (SSCs) into mature spermatozoa.

FIG. 2 diagrammatically depicts the stages during differentiation of adult spermatogonial stem cells (SSCs) into mature spermatozoa.

FIG. 3 depicts a diagrammatic overview of a process of isolating and purifying differentiating SPGs from harvested testicular tissue. In brief, testicular tissue was processed into a single cell suspension, followed by MACS enrichment of KIT+ cells and culturing in meiotic entry (ME) media.

FIG. 4 are photomicrographs depicting emergence of primary spermatocytes after 7 days in culture as identified by the spermatocyte marker SYCP3 (red), germ cell marker DDX4 (green) and DNA marker Hoechst 33342 (Blue).

FIG. 5 are plots quantifying the results shown in FIG. 39. The number and percentage of SYCP3+DDX4+primary spermatocytes increase over time in ME culture are shown.

FIG. 6 Diagrammatic representation of the stages of meiosis of human germ cells during meiosis.

FIG. 7. Violin plots of gene expression across spermatogenesis. Differential expression analyses of single cell RNA sequencing data produced a set of target genes to allow for interrogation of transcriptional changes during in vitro spermatogenesis.

FIG. 8A Fold change expression of genes associated with spermatogenesis in Bulk cells, Kit+ cells Day 1, Day3 and Day 7 after exposure to Meiotic Entry (ME) media.

FIG. 8B Comparison of fold change expression of genes associated with spermatogenesis in Kit+sorted cells in Day 1 and Day 7 after ME media exposure. Gene list reads from left to right for each condition. 2-way ANOVA, *p<0.05, ** p<0.01, *** p<0.001, **** p<0.0001

FIG. 9 are photomicrographs of meiotic chromosome spreads of spermatocytes. EdU (green), SYCP3 (yellow), H2AX (red), and DNA (DAPI, blue in merged images).

FIG. 10 depicts microfluidic spiral channel separated fluorescent particles based on size. Panel A Left: Commercially available microfluidic spiral channel with 4 distinct spiral channel geometries. Syringe pump is connected to sorting unit 2 inlet (arrow) and sorted particles are collected from outlets (Outlet IDs listed). Right: Image of sorting unit #2 outlets. Outlet ID #1 is the outermost outlet and ID #8 is the inner-most outlet. Panel B: Fluorescent microscopy of focused fluorescent particles at sorting unit #2 outlets (6 μm blue, 10 μm green, 15 μm red). Outlet IDs listed. Particles were run at 1.5 mL/minute. Panel C: Particles were harvested from each outlet (labeled on image) and imaged with fluorescent microscopy.

FIG. 11A Spiral channel microfluidic approach enriches haploid round spermatids by separating cells based on size. Cell diameter measurements (μm) of each outlet immediately after cell sorting, with smaller cells observed in outlets 4 and 5 and larger cells observed in outlets 7 and 8. One-way ANOVA, **** p<0.0001, n=2.

FIG. 11B Spiral channel microfluidic approach enriches haploid round spermatids by separating cells based on size. Cells were cultured overnight and stained with ACRV1, a haploid round spermatid marker. Fluorescent microscopy was used to identify ACRV1+ cells. One-way ANOVA, **** p<0.0001, n=2.

FIG. 12A The formation of human round spermatids in vitro. Representative fluorescent microscopy images of DDX4/ACRV1 staining on day 1 and day 10. Increased ACRV1+/DDX4-cells on day 10 (grey arrow heads show examples) demonstrate increased round spermatids after 10 days in ME media.

FIG. 12B The formation of human round spermatids in vitro. Quantification of ACRV1+ cells across listed conditions over the course of 20 days. All conditions result in increased round spermatid formation after 10 days of culture in vitro. n=1 in technical duplicate.

FIG. 13 Fold change in expression of genes associated with spermatogenesis in Kit+ cells Day 1 after exposure to Meiotic Entry (ME) media and Day 7 after exposure to MP media.

FIG. 14A. Representative immunofluorescence images showing human differentiating spermatogonia (dSPGs) after culture in meiotic entry conditions. Cells were stained for DNA (blue), γH2AX (red), EdU (green), and HORMAD1 (white) to identify cells that have initiated meiotic prophase. Arrows indicate EdU+ primary spermatocytes positive for early meiotic markers, demonstrating successful meiotic entry under optimized culture conditions. Arrows: IVS-derived primary spermatocytes derived from dSPGs.

FIG. 14B. Quantification of meiotic entry efficiency in human dSPGs cultured under different conditions. Bar plot shows the percentage of cells entering early meiosis (EdU+ and positive for meiotic markers) for meiotic entry media (ME; control) and for the optimized meiotic entry media of the instant disclosure. Data represent mean±SEM from independent donor samples, demonstrating a substantial increase in meiotic entry efficiency with the optimized conditions compared to control.

FIG. 14C. Quantification of meiotic entry efficiency in human dSPGs cultured under different conditions. Bar plot shows the percentage of cells entering early meiosis (EdU+ and positive for meiotic markers) for meiotic entry media (ME; control) and for meiotic entry media supplemented with insulin and IWR-endo-1, a WNT regulator. Data represent mean±SEM from independent donor samples, demonstrating a substantial increase in meiotic entry efficiency with the optimized conditions compared to control.

FIG. 15 is a plot showing the results of a screen of reagents used to identify compounds that facilitate entry of spermatogonial stem cells (SSCs) into meiosis. Compounds of interest are highlighted with orange boxes.

FIG. 16. Representative immunofluorescence image showing a human spermatocyte at the pachytene stage of meiotic prophase I, after culture dSPGs in meiotic progression conditions. Cells were stained for DNA (blue), γH2AX (red), EdU (green), and HORMAD1 (white) and quantification demonstrate that co-culture of human differentiating spermatogonia (dSPGs) with MEF feeder cells enables progression beyond early meiotic prophase. Cells were stained for EdU (green), HORMAD1 (white), and gH2AX to identify late-meiotic stage spermatocytes.

FIG. 17. Representative immunofluorescence images show cells stained for DNA (blue), EdU (green), ACRV1 (red), and DDX4 (white), highlighting the emergence of EdU+ACRV1+DDX4+haploid spermatids. These results demonstrate successful completion of meiosis and production of haploid germ cells under the optimized culture system utilizing nutrient-rich progression media and MEF feeder cells.

FIG. 18A shows representative photomicrographs of round spermatids cultured in SP medium for 1 day and 6 days. Arrows indicate cells exhibiting flagella.

FIG. 18B is a bar plot quantifying the production of flagellar cells in cultures of round spermatids grown in base medium, SP1 medium, SP2 medium, and SP3 medium. Arrows indicate cells exhibiting flagella. Black arrows indicate spermatids with flagella.

FIG. 19. Sequential images of a human spermatozoon generated in vitro, captured at different time points following the addition of Pentoxyfiline. The images show the changing position of the flagellum over time, providing visual evidence of active flagellar movement and motility in the in vitro-derived spermatozoon.

FIG. 20. Schematic overview of the sequential in vitro differentiation protocol for human spermatogenesis, illustrating the stepwise progression from dSPGs and SSCs through meiotic entry, meiotic progression, meiotic completion, and spermiogenesis to the formation of flagellated spermatids then spermatozoa. The diagram summarizes the media formulations and transitions required to recapitulate the full developmental pathway from dSPGs and SSCs to spermatozoa in vitro.

### DETAILED DESCRIPTION

The present disclosure encompasses methods and cell culture systems for supporting the complete in vitro development of human testicular germ cells through all major stages of spermatogenesis. The invention is based on the discovery that distinct and sequentially tailored culture conditions are required for each critical transition, including meiotic entry of dSPGs and SSCs, through to meiotic progression, meiotic completion, and spermiogenesis. Through extensive systematic experimentation and optimization, new media formulations and combinations of supplements were identified that enable, for the first time, the efficient and reproducible differentiation of human dSPGs and SSCs to spermatozoa in vitro. The invention further provides compositions and protocols for each stage, including nutrient-restricted and nutrient-rich media, specific growth factors, hormones, and signaling pathway modulators, as well as the use of feeder cells and conditioned media where appropriate. These discoveries enable the faithful recapitulation of human spermatogenesis in vitro and provide powerful tools for research, infertility treatment, and the development of assisted reproductive technologies.

### I. Culture Systems

One aspect of the instant disclosure encompasses sequential and stage-specific cell culture systems that enable the complete in vitro differentiation of human dSPGs and SSCs through multiple defined steps, including induction of meiotic entry, progression through meiotic prophase and completion of meiosis to form haploid round spermatids, and subsequent spermiogenesis resulting in the production of elongated, flagellated spermatids and then spermatozoa.

### (a) Spermatogenesis

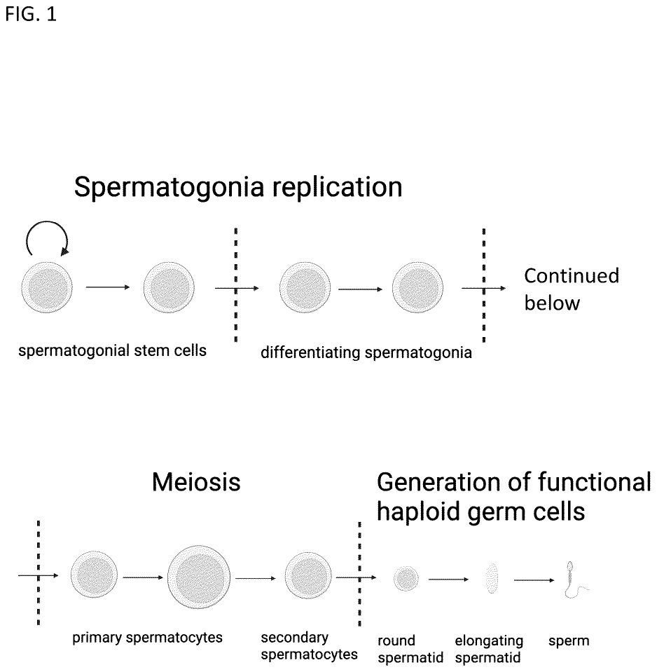

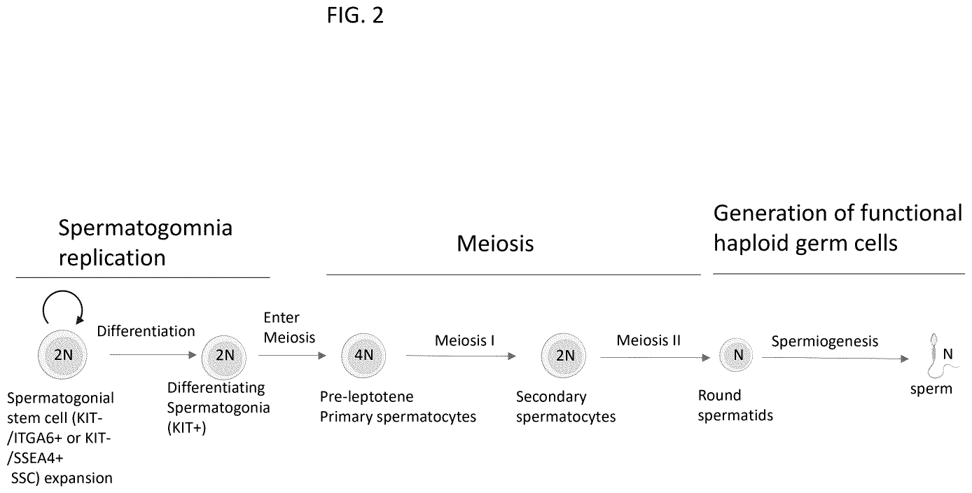

Mammalian spermatogenesis is a complex, multi-stage developmental process in which SSCs undergo a series of tightly regulated transitions to ultimately form mature spermatozoa. In humans, this process involves the sequential progression of SSCs to dSPGs through meiotic entry, meiotic progression to primary and secondary spermatocytes, completion of meiosis to generate haploid round spermatids, and subsequent spermiogenesis resulting in the formation of elongated, flagellated spermatids, and spermatozoa (see FIG. 1 and FIG. 2). Each of these stages is characterized by distinct cellular and molecular events, including chromatin remodeling, homologous chromosome pairing and recombination, and dramatic morphological changes.

The term “differentiating spermatogonia” (dSPGs) refers to male germ cells that have committed to the spermatogenic lineage and are poised to enter meiosis. dSPGs are developmentally downstream of spermatogonial stem cells (SSCs), which are the most primitive, undifferentiated spermatogonia responsible for the long-term maintenance and self-renewal of the germline. SSCs are defined by their capacity for self-renewal and the ability to repopulate the seminiferous epithelium following transplantation, whereas dSPGs have lost self-renewal capacity and are committed to differentiation, ultimately giving rise to primary spermatocytes. SSCs are typically quiescent or slowly cycling, reside on the basement membrane of the seminiferous tubules, and are capable of long-term self-renewal. In contrast, dSPGs are more proliferative, form chains or clusters as they undergo transit-amplifying divisions, and are committed to differentiation and meiotic entry.

The distinction between SSCs and dSPGs is reflected in their marker expression profiles. SSCs are characterized by the expression of markers such as GFRα1 (GDNF family receptor alpha-1), ID4 (Inhibitor of DNA binding 4), UTF1 (Undifferentiated embryonic cell transcription factor 1), PLZF (Promyelocytic leukemia zinc finger, also known as ZBTB16), SALL4, LIN28A, TSPAN33, PIWIL4, EGR4, MSL3, TSPAN8, ITGA6 (CD49f), THY1 (CD90), and SSEA4. These markers are associated with the undifferentiated, self-renewing state of SSCs. As SSCs transition to dSPGs, they lose expression of many of these stem cell markers and begin to express markers indicative of differentiation and commitment to meiosis. dSPGs are identified by the expression of KIT (CD117), which is a canonical marker of differentiating spermatogonia and is not expressed in SSCs. Additional markers of dSPGs include STRA8 (Stimulated by retinoic acid gene 8), which is upregulated in response to retinoic acid and associated with meiotic initiation, as well as DMRT1, SOHLH1, SOHLH2, SYCP3 (at low levels in late dSPGs), MK167 (a proliferation marker), CCNE1, DMRTB1, and DAZL. The expression of NANOS2 is downregulated as cells differentiate, and DAZL is present in both undifferentiated and differentiating spermatogonia but persists in dSPGs.

The transition from SSCs to dSPGs is marked by a shift in marker expression and cellular behavior, which can be reliably identified using the markers listed above. The ability to distinguish between these populations is critical for understanding and recapitulating the stages of human spermatogenesis in vitro, as described in the present disclosure.

dSPGs then progress to meiosis to generate round spermatids, passing through a series of tightly regulated developmental checkpoints that ensure the fidelity of gamete formation. The first major checkpoint is meiotic entry, where differentiating spermatogonia initiate meiosis and become primary spermatocytes. As these cells enter meiotic prophase I, they encounter the pachytene checkpoint, a surveillance mechanism that monitors the successful pairing (synapsis) and recombination of homologous chromosomes. Failure to achieve proper synapsis or repair of programmed DNA double-strand breaks at this stage results in arrest and elimination of defective cells. Additional checkpoints occur at the transitions from metaphase I to anaphase I and from metaphase II to anaphase II, where the correct alignment and segregation of chromosomes and sister chromatids are verified by the spindle assembly apparatus. Only cells that successfully navigate these checkpoints complete both meiotic divisions to form haploid round spermatids, which then undergo spermiogenesis to become elongated, flagellated spermatids, then spermatozoa. These developmental checkpoints are essential for maintaining genomic integrity and ensuring the production of functional spermatozoa.

Following the completion of meiosis and the formation of haploid round spermatids, these cells undergo spermiogenesis, the final phase of spermatogenesis. Spermiogenesis is a highly orchestrated process during which round spermatids undergo extensive morphological and biochemical changes to become elongated, flagellated spermatids, and spermatozoa. This transformation involves nuclear condensation, acrosome formation, cytoplasmic reduction, and the development of the flagellum, which is essential for sperm motility. Throughout spermiogenesis, specific molecular markers and structural changes can be used to identify the progression of spermatids, including the appearance of acrosomal proteins, changes in chromatin packaging, and the emergence of flagellar structures. The successful completion of spermiogenesis is critical for the production of mature, motile spermatozoa capable of fertilization, and defects in this process can result in abnormal sperm morphology and impaired fertility.

The inventors discovered that successful in vitro human spermatogenesis required the development and application of three distinct, sequential culture conditions, each tailored to a specific stage of germ cell development. First, a unique set of conditions was established to induce meiotic entry, enabling differentiating spermatogonia (dSPGs) to initiate meiosis and become primary spermatocytes. Next, a second, nutrient-rich culture environment was optimized to support meiotic progression and completion, allowing primary spermatocytes to advance through the critical stages of prophase I, complete both meiotic divisions, and form haploid round spermatids. Finally, a third specialized medium was developed to promote spermiogenesis, facilitating the transformation of round spermatids into elongated, spermatozoa. Each of these steps required precise modulation of the culture environment, with specific combinations of nutrients, growth factors, and signaling molecules, underscoring the necessity of stage-specific conditions to faithfully recapitulate the entire process of human spermatogenesis in vitro.

Accordingly, aspects of the instant disclosure encompass a first culture system for inducing meiotic entry of dSPGs up to, but not including, metaphase; a second culture system for supporting the progression and completion of meiosis to generate round spermatids; and a third culture system for promoting spermiogenesis from round spermatids to spermatozoa. The first, second, and third culture systems are described in Sections I (b), I (c), and I(d), respectively. By combining these systems sequentially, the inventors were able to recapitulate the entire process of human spermatogenesis in vitro, as described in Section I (e).

### (b) Meiotic Entry

One aspect of the instant disclosure encompasses a cell culture system for inducing entry of human dSPGs or SSCs into meiosis in vitro. The system comprises a first cell culture medium for culturing human dSPGs or SSCs for a first culture period.

The dSPGs or SSCs can be isolated directly from human testicular tissue, which can be obtained from a living or cadaveric donor using methods recognized by individuals of skill in the art. Alternatively, the human dSPGs or SSCs can be derived from cells previously cultured in vitro. In some aspects, the cells are freshly isolated or can be obtained from cryopreserved or previously frozen testicular tissue or cell preparations. The invention is compatible with a wide range of cell sources, including cells from prepubertal or adult individuals, fertile or infertile subjects, and tissue or cells that have been stored for future use, such as for fertility preservation prior to gonadotoxic treatments.

In some aspects, the cells are human SSCs. In some aspects, the cells are human SSCs isolated from testicular tissue. In some aspects, the cells are human SSCs from cells previously cultured in vitro. In other aspects, the cells are human dSPCs. In some aspects, the cells are human dSPCs isolated from testicular tissue. In some aspects, the cells are human dSPCs from cells previously cultured in vitro.

The first culture medium comprises a nutrient-rich basal medium, wherein the nutrient-rich basal medium is present at a sufficiently low concentration to provide a nutrient-restricted environment. As used herein, a “nutrient-restricted medium” refers to a cell culture medium in which the concentration of a nutrient-rich basal medium is substantially reduced relative to standard culture conditions, typically by dilution with a buffered salt solution. Nutrient-restricted media comprise significantly lower concentrations of amino acids, vitamins, glucose, and other nutrients compared to conventional complete media. Without wishing to be bound by theory, this environment is designed to limit nutrient availability and thereby influence cell signaling, differentiation, or developmental processes.

As used herein, the term “nutrient-rich basal medium” refers to a cell culture medium formulation that provides a comprehensive supply of essential nutrients required to support the growth, viability, proliferation, and differentiation of mammalian cells in vitro. A nutrient-rich basal medium typically contains a balanced mixture of amino acids, vitamins, inorganic salts, glucose or other energy sources, and may also include additional components such as nucleosides, lipids, and trace elements. These media are formulated to mimic the nutrient composition of physiological fluids and provide the necessary building blocks for cellular metabolism, biosynthesis, and maintenance of cellular homeostasis.

Nutrient-rich basal media are known by individuals of skill in the art. Examples of nutrient-rich basal media include, but are not limited to, Minimum Essential Medium Alpha (αMEM), Advanced Protein-free Eukaryotic cell Line medium 2 (APEL2), Medium 199 (M199), and Dulbecco's Modified Eagle Medium/Nutrient Mixture F-12 (DMEM/F12). Such media are distinguished from simple buffered salt solutions (such as Earle's Balanced Salt Solution (EBSS) or Hanks' Balanced Salt Solution (HBSS)), which lack amino acids, vitamins, and energy sources and are not sufficient to support long-term cell growth or proliferation on their own.

In some aspects, the nutrient-rich basal medium of a first culture medium of the instant disclosure is Minimum Essential Medium Alpha (αMEM), Advanced Protein-free Eukaryotic cell Line medium 2 (APEL2), Medium 199 (M199), Dulbecco's Modified Eagle Medium/Nutrient Mixture F-12 (DMEM/F12), or any combination thereof. In some aspects, the nutrient-rich basal medium is αMEM.

A nutrient-rich basal medium is generally used at concentrations sufficient to provide optimal nutrient availability for mammalian cell culture, often comprising 80 to 100% of the total medium volume in standard culture conditions. The specific formulation and concentration of each component may vary depending on the cell type and application, but the defining characteristic is the presence of a full complement of nutrients necessary for robust cellular function. A first culture medium of the instant disclosure is a nutrient-restricted medium, comprising a sufficiently low concentration to provide a nutrient restriction medium. As used herein, the terms “nutrient-restricted media” or “nutrient restriction media” are used interchangeably and refer to a cell culture medium in which the concentration of a nutrient-rich basal medium is substantially reduced compared to standard culture conditions. This reduction is typically achieved by diluting the nutrient-rich basal medium with a buffered salt solution, such as Earle's Balanced Salt Solution (EBSS), Hanks' Balanced Salt Solution (HBSS), or phosphate-buffered saline (PBS), so that the nutrient-rich basal medium constitutes a minor fraction of the total medium volume. As a result, the overall concentrations of amino acids, vitamins, glucose, and other nutrients are significantly lower than in conventional complete media, creating an environment of limited nutrient availability that can influence cell signaling, differentiation, or developmental processes.

A nutrient restricted first culture medium can comprise a nutrient-rich basal medium at concentrations ranging from about 1% to about 60%, from about 5% to about 40%, from about 5% to about 30%, from about 5% to about 20%, from about 5% to about 15%, or from about 8% to about 12%. In some aspects, the first culture medium comprises αMEM at a concentration ranging from about 8% to about 12%. In some aspects, the sufficiently low concentration of nutrient rich basal media is achieved by diluting the nutrient-rich basal medium with EBSS, HBSS, PBS, or any combination thereof. In some aspects, the first culture medium comprises a sufficiently low concentration αMEM diluted in EBSS to provide a nutrient restriction medium. In some aspects, the first culture medium comprises αMEM at a concentration ranging from about 8% to about 12% diluted in about 88% to about 92% EBSS.

A first medium of the instant disclosure also comprises a serum or serum substitute. Serums or serum substitutes are known by individuals of skill in the art. A serum or serum substitute refers to a component of cell culture medium that provides essential growth factors, hormones, proteins, lipids, vitamins, trace elements, and other nutrients necessary to support the survival, proliferation, and differentiation of cells in vitro. Serum is typically derived from animal sources, such as fetal bovine serum (FBS), and contains a complex mixture of undefined biological components. Serum substitutes, in contrast, are chemically defined or semi-defined formulations designed to replace animal-derived serum, providing a more controlled and reproducible environment for cell culture.

In some aspects, a first culture medium of the instant disclosure comprises an animal-derived serum. Non-limiting examples of animal-derived serums commonly used in vitro include fetal bovine serum (FBS), newborn calf serum (NCS), adult bovine serum, horse serum, goat serum, rabbit serum, porcine serum, and human serum. These serums can be collected from the blood of the respective animal source and processed to provide a rich mixture of growth factors, hormones, proteins, and other nutrients that support cell growth and viability in culture. Concentrations of animal derived serum are recognized by individuals of skill in the art and can range from about 1% to about 50%, from about 2% to about 40%, from about 3% to about 30%, or from about 4% to about 25.

In some aspects, a first culture medium of the instant disclosure comprises a serum substitute. Non-limiting examples of serum substitutes include Knockout Serum Replacement (KSR), B27 supplement, N2 supplement, Serum Replacement 1 (SR1), Serum Replacement 2 (SR2), StemPro Serum Replacement, ITS or ITS-X supplement (insulin, transferrin, selenium), Essential 8 (E8) supplement, TeSR™-E8™, mTeSR™1, chemically defined lipid concentrate, and any combination thereof.

In some aspects, a first culture medium of the instant disclosure comprises a serum or serum substitute selected from KSR, B27 supplement, N2 supplement, SR1, SR2, StemPro Serum Replacement, ITS or ITS-X supplement, E8 supplement, TeSR™-E8™ mTeSR™1, chemically defined lipid concentrate, or any combination thereof. A nutrient restricted first culture medium of the instant disclosure can comprise a nutrient-rich basal medium at concentrations ranging from about 0.01% to about 30%, from about 0.1% to about 20%, from about 0.1% to about 10%, from about 0.1% to about 5%, or from about 0.1% to about 2%. In some aspects, a first culture medium of the instant disclosure comprises KSR serum substitute. In some aspects, the first culture medium comprises KSR at a concentration ranging from about 0.1% to about 2%. In some aspects, the first culture medium comprises KSR at a concentration of about 1%.

The first culture medium further comprises a retinoid or retinoic acid receptor agonist. As used herein, a “retinoid or retinoic acid receptor agonist” refers to any natural or synthetic compound that is capable of binding to and activating retinoic acid receptors (RARs) or retinoid X receptors (RXRs), thereby modulating gene expression and cellular processes regulated by retinoic acid signaling pathways. Retinoids include vitamin A (retinol), its natural metabolites (such as retinal and retinoic acid), and a wide range of structurally related synthetic analogs. Retinoic acid receptor agonists specifically activate RARs or RXRs and can promote cellular differentiation, proliferation, and developmental processes, including the induction of meiosis in germ cells. Non-limiting examples of retinoids or retinoic acid receptor agonists include all-trans retinoic acid, 9-cis retinoic acid, 13-cis retinoic acid, retinol, retinal, retinyl acetate, retinyl palmitate, AM580, TTNPB, adapalene, tazarotene, bexarotene, and acitretin.

In some aspects, the retinoid or retinoic acid receptor agonist is all-trans retinoic acid, 9-cis retinoic acid, 13-cis retinoic acid, retinol, retinal, retinyl acetate, retinyl palmitate, AM580, TTNPB, adapalene, tazarotene, bexarotene, acitretin, or any combination thereof. In some aspects, the retinoid or retinoic acid receptor agonist is retinoic acid. The retinoic acid can be used at concentrations ranging from about 0.01 uM to about 10 uM, from about 0.1 uM to about 9 uM, from about 1 uM to about 8 uM, or from about 4 uM to about 6 uM. In some aspects, the first culture medium comprises retinoic acid at a concentration ranging from about 4 uM to about 6 uM.

The first culture medium also comprises a TGF-β superfamily ligand. As used herein, a “TGF-β superfamily ligand” refers to any member of the transforming growth factor beta (TGF-β) superfamily of cytokines, which are a large group of structurally related proteins that regulate a wide variety of cellular processes, including cell growth, differentiation, apoptosis, and development. TGF-β superfamily ligands include, but are not limited to, the TGF-β isoforms (TGF-β1, TGF-β2, TGF-β3), activins (such as activin A, activin B, activin AB, activin C, activin E), bone morphogenetic proteins (BMPs, such as BMP1, BMP2, BMP3, BMP4, BMP5, BMP6, BMP7, BMP8a, BMP8b, BMP9, BMP10, BMP11, BMP12, BMP13, BMP14, BMP15), growth differentiation factors (GDFs, such as GDF5, GDF6, GDF7, GDF9, GDF11, myostatin/GDF8), nodal, and other related proteins. These ligands exert their effects by binding to specific cell surface receptors and activating intracellular signaling pathways that are critical for germ cell development and spermatogenesis. In some aspects, the TGF-β superfamily ligand is activin A, activin B, activin AB, activin C, activin E, transforming growth factor beta 1 (TGF-β1), transforming growth factor beta 2 (TGF-β2), transforming growth factor beta 3 (TGF-β3), bone morphogenetic proteins (BMPs), growth differentiation factor 5 (GDF5), growth differentiation factor 6 (GDF6), growth differentiation factor 7 (GDF7), growth differentiation factor 9 (GDF9), growth differentiation factor 11 (GDF11), nodal, myostatin (GDF8), or any combination thereof.

In some aspects, the TGF-β superfamily ligand is a BMP and an activin. In some aspects, the BMP is BMP1, BMP2, BMP3, BMP4, BMP5, BMP6, BMP7, BMP8a, BMP8b, BMP9, BMP10, BMP11, BMP12, BMP13, BMP14, BMP15, or any combination thereof. In some aspects, the BMP is BMP2, BMP4 and BMP7. In some aspects, the first culture medium comprises BMP2, BMP4 and BMP7 each at a concentration ranging from about 15 ng/ml to about 25 ng/ml and activin A at a concentration ranging from about 90 ng/mL to about 110 ng/ml. In some aspects, the first culture medium comprises BMP2, BMP4 and BMP7 each at a concentration ranging from about 15 ng/ml to about 25 ng/ml.

In some aspects, the activin is activin A, activin B, activin AB, activin C, activin E, or any combination thereof. In some aspects, the activin is activin A. In some aspects, the first culture medium comprises activin A at a concentration ranging from about 90 ng/ml to about 110 ng/ml. In some aspects, the first culture medium comprises BMP2, BMP4 and BMP7 each at a concentration ranging from about 15 ng/ml to about 25 ng/ml and activin A at a concentration ranging from about 90 ng/ml to about 110 ng/ml.

A first culture medium of the instant disclosure comprises insulin. Through extensive experimentation described in the examples herein below, the inventors discovered that the addition of insulin significantly improved the efficiency of entry of cells into meiosis. The insulin in the first culture medium can be human insulin, porcine insulin, bovine insulin, insulin-like growth factor 1 (IGF-1), insulin-like growth factor 2 (IGF-2), or any combination thereof. In some aspects, the insulin in the first culture medium is human insulin. Insulin can be used in culture media at concentrations ranging from about 1 ug/mL to about 100 ug/mL, from about 10 ug/mL to about 80 ug/mL, from about 20 ug/mL to about 70 ug/mL, from about 30 g/mL to about 60 ug/mL, or from about 40 ug/mL to about 60 ug/mL. In some aspects, the first culture medium comprises insulin at a concentration ranging from about 40 ug/mL to about 60 ug/mL. In some aspects, the first culture medium comprises insulin at a concentration of about 50 ug/mL.

Through extensive experimentation described in the examples herein below, the inventors also discovered that the addition of a WNT signaling pathway regulator significantly improved the efficiency of entry of cells into meiosis. Accordingly, the first culture medium of the instant disclosure also comprises a WNT signaling pathway regulator. The WNT signaling pathway is a highly conserved cellular signaling cascade that plays a critical role in regulating cell fate, proliferation, differentiation, and tissue homeostasis. In the context of germ cell development and spermatogenesis, WNT signaling influences the balance between self-renewal and differentiation of germ cells. WNT pathway regulators are compounds that modulate the activity of this pathway, either by activating (agonists) or inhibiting (antagonists) WNT signaling.

Non-limiting examples of WNT pathway regulators include small molecule inhibitors and protein antagonists such as IWR-endo-1, DKK1, XAV939, ICG-001, C59, LGK974, PRI-724, PNU-74654, FH535, Wnt-C59, DKK2, and sFRP1. In some aspects, the first culture medium of the instant disclosure comprises a WNT pathway inhibitor. Non-limiting examples of WNT pathway inhibitors include IWR-endo-1, DKK1, XAV939, ICG-001, C59, LGK974, PRI-724, PNU-74654, FH535, Wnt-C59, DKK2, and sFRP1. In some aspects, the WNT pathway inhibitor is IWR-endo-1.

A first culture medium of the instant disclosure can comprise IWR-endo-1 at concentrations ranging from about 0.1 uM to about 10 UM, from about 1 uM to about 8 UM, from about 2 uM to about 6 UM, or from about 2 uM to about 4 uM. In some aspects, the first culture medium comprises IWR-endo-1 at a concentration ranging from about 2 uM to about 4 uM. In some aspects, the first culture medium comprises IWR-endo-1 at a concentration of about 5 uM.

In some aspects, the first culture medium comprises αMEM, KSR, BMP2, BMP4, BMP7, retinoic acid, activin A, insulin, and IWR-endo-1. In some aspects, the first culture medium comprises αMEM at a concentration ranging from about 8% to about 12%, KSR at a concentration ranging from about 0.1% to about 2%, BMP2 at a concentration ranging from about 15 ng/ml to about 25 ng/ml, BMP4 at a concentration ranging from about 15 ng/ml to about 25 ng/ml, BMP7 at a concentration ranging from about 15 ng/ml to about 25 ng/ml, retinoic acid at a concentration ranging from about 4 uM to about 6 uM, activin A at a concentration ranging from about 90 ng/ml to about 110 ng/ml, insulin at a concentration ranging from about 40 ug/mL to about 60 ug/mL, and IWR-endo-1 at a concentration ranging from about 2 uM to about 4 uM.

As it will be recognized by individuals of skill in the art, the first culture medium can further comprise one or more additional common cell culture ingredients selected from the group consisting of a diluent or buffered salt solution such as Earle's Balanced Salt Solution (EBSS), Hanks' Balanced Salt Solution (HBSS), or phosphate-buffered saline (PBS); an antibiotic or antimicrobial agent such as penicillin-streptomycin (Pen/Strep), gentamicin, or amphotericin B; a pH buffer such as HEPES or sodium bicarbonate; and other standard cell culture supplements. In some aspects, the first culture medium further comprises EBSS. In some aspects, the first culture medium further comprises EBSS at a concentration ranging from about 40% to about 99%, from about 60% to about 95%, from about 70% to about 95%, from about 80% to about 95%, or from about 88% to about 92%. In some aspects, the first culture medium comprises αMEM at a concentration ranging from about 8% to about 12% diluted in about 88% to about 92% EBSS.

In some aspects, the first cell culture medium further comprises Pen/Strep. In some aspects, the first cell culture medium further comprises Pen/Strep at a concentration ranging from bout 0.5% to about 1.5%.

In some aspects, the first cell culture medium further comprises EBSS and Pen/Strep. In some aspects, the first cell culture medium further comprises Pen/Strep at a concentration ranging from bout 0.5% to about 1.5%.

The first culture medium of the instant disclosure can be used to culture dSPGs or SSCs for a first period of time. The first period of time can be sufficient to allow the cells to enter meiosis. As described in the examples herein below, the inventors tried a number of culture periods to identify culture periods sufficient to allow the cells to enter meiosis. The inventors discovered that the first culture period can range from about 12 hrs to about 5 days or longer. For instance, the first culture period can be 12, hrs, 1 day, 2 days, 3 days, 4 days, 5 days, or longer. In some aspects, the first culture period ranges from about 12 hrs to about 5 days. In some aspects, the first culture period is about 1 day to about 4 days. In some aspects, the first culture period is about 1 day. In some aspects, the first culture period is about 2 day. In some aspects, the first culture period is about 2 day. In some aspects, the first culture period is about 4 days.

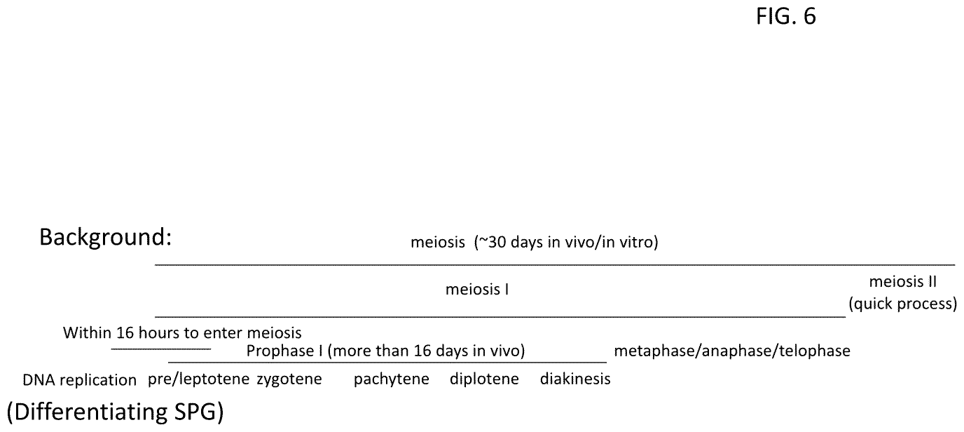

Entry of human dSPGs and SSCs into meiosis is evidenced by the appearance of spermatocytes arrested before the pachytene stage of meiosis I, as characterized by the presence, absence, or localization of cell markers indicative of cells before the pachytene stage, after culturing the dSPGs or SSCs in the first culture medium for the first culture period (See FIG. 6 for meiotic stages of development of meiosis of human germ cells). Methods of determining the stage of development of a germ cell, including entry of a dSPG or SSC into meiosis are known to individuals of skill in the art. In general, the presence of spermatocytes arrested before the pachytene stage of meiosis I can be determined by the presence of cells characterized by the presence, absence, or localization of cell markers indicative of cells before the pachytene stage, after culturing the dSPGs or SSCs in the first culture medium for the first culture period. For instance, the appearance of spermatocytes arrested before the pachytene stage of meiosis I can be characterized by EdU incorporation indicating DNA replication; the presence of HORMAD1 on unsynapsed chromosome axes; presence of γH2AX diffusely throughout the nucleus, prior to its restriction to the sex body; presence of SYCP1 as punctate or partial linear staining, prior to full synapsis; presence of DMC1 and/or RAD51 as nuclear foci, with the number of foci decreasing as cells progress to pachytene; presence of MEIOB, REC8, PRDM9, or STRA8, with STRA8 expression being downregulated as cells progress through prophase I; absence, reduction, or relocalization of HORMAD1 and γH2AX from autosomes, or disappearance of DMC1 and RAD51 foci, indicating progression beyond the pre-pachytene stage; or any combination thereof. In some aspects, entry of the human dSPGs or SSCs into meiosis is evidenced by the appearance of spermatocytes arrested before the pachytene stage of meiosis I characterized by EdU incorporation indicating DNA replication; the presence of HORMAD1 on unsynapsed chromosome axes and presence of γH2AX diffusely throughout the nucleus, prior to its restriction to the sex body; absence, reduction, or relocalization of HORMAD1 and γH2AX from autosomes; or any combination thereof.

In some aspects, a cell culture system for entry of human dSPGs or SSCs into meiosis in vitro comprises a first cell culture medium for culturing human dSPGs or SSCs for about 4 days. The first cell culture medium can comprise: EBSS at a concentration of about 90% (v/v); αMEM at a concentration of about 10% (v/v); KSR at a concentration of about 1% (v/v); Pen-Strep at a concentration of about 1%; BMP2, BMP4, and BMP7 each at a concentration of about 20 ng/ml; retinoic acid at a concentration of about 5 uM; activin A at a concentration of about 100 ng/ml; insulin at a concentration of about 50 ug/mL; and IWR-endo-1 at a concentration of about 3 uM. In some aspects, entry of the human dSPGs or SSCs into meiosis is evidenced by the appearance of spermatocytes arrested before the pachytene stage of meiosis I after culturing the dSPGs or SSCs in the cell culture system during the first culture period, wherein the spermatocytes are characterized by the presence of HORMAD1 on unsynapsed chromosome axes and presence of γH2AX diffusely throughout the nucleus, prior to its restriction to the sex body; absence, reduction, or relocalization of HORMAD1 and γH2AX from autosomes; or any combination thereof.

### (c) Meiotic Progression and Completion

Another aspect of the instant disclosure encompasses a second cell culture system for supporting the progression of human spermatocytes arrested before the pachytene stage of meiotic prophase I through completion of meiosis and formation of round spermatids in vitro. Human spermatocytes arrested before the pachytene (pre-pachytene cells) stage of meiotic prophase I can be isolated directly from human testicular tissue, which can be obtained from a living or cadaveric donor using methods recognized by individuals of skill in the art. Alternatively, the human pre-pachytene cells can be derived from cells previously cultured in vitro. In some aspects, the cells are freshly isolated or can be obtained from cryopreserved or previously frozen testicular tissue or cell preparations. The invention is compatible with a wide range of cell sources, including cells from prepubertal or adult individuals, fertile or infertile subjects, and tissue or cells that have been stored for future use, such as for fertility preservation prior to gonadotoxic treatments.

In some aspects, the cells are human pre-pachytene spermatocytes are human pre-pachytene spermatocytes isolated from testicular tissue. In some aspects, the human pre-pachytene spermatocytes are derived from cells previously cultured in vitro in a first culture system as described in Section I(b) herein above, using methods described in Section III herein below.

The second cell culture system comprises a second cell culture medium for culture of human spermatocytes arrested before the pachytene stage of meiotic prophase I; and feeder cells.

### A. Second Cell Culture Medium

The second cell culture system comprises a second cell culture medium for culture of human spermatocytes arrested before the pachytene stage of meiotic prophase I for a second culture period and feeder cells. The second culture medium comprises a nutrient-rich basal medium, wherein the nutrient-rich basal medium is present at a sufficiently high concentration to provide a nutrient-rich environment. As used herein, a “nutrient-rich medium” refers to a cell culture medium in which the concentration of a nutrient-rich basal medium is comparable to or greater than standard culture conditions. Such media contain high concentrations of amino acids, vitamins, glucose, and other nutrients necessary to support robust cell growth, viability, and differentiation.

Nutrient rich basal medium can be as described in Section I(b) herein above. In some aspects, the nutrient-rich basal medium of a second culture medium of the instant disclosure is Minimum Essential Medium Alpha (αMEM), Advanced Protein-free Eukaryotic cell Line medium 2 (APEL2), Medium 199 (M199), Dulbecco's Modified Eagle Medium/Nutrient Mixture F-12 (DMEM/F12), or any combination thereof. In some aspects, the nutrient-rich basal medium is αMEM.

A nutrient-rich basal medium is generally used at concentrations sufficient to provide optimal nutrient availability for mammalian cell culture, often comprising 80 to 100% of the total medium volume in standard culture conditions. The specific formulation and concentration of each component may vary depending on the cell type and application, but the defining characteristic is the presence of a full complement of nutrients necessary for robust cellular function. A second culture medium of the instant disclosure comprises a sufficiently high concentration to provide a nutrient rich medium. As used herein, the term “nutrient-rich media” refers to a cell culture medium in which the concentration of a nutrient-rich basal medium is comparable to or greater than that used in standard cell culture conditions. As it will be recognized by individuals of skill in the art, a nutrient rich medium can comprise about 70% to about 100% of the total medium volume. For instance, it can comprise about 70%, 75%, 80%, 85%, 90%, 95% or 100% of the total medium volume. Such media provide high levels of amino acids, vitamins, glucose, and other essential nutrients required to support robust cell growth, viability, proliferation, and differentiation.

Accordingly, a nutrient rich second culture medium can comprise a nutrient-rich basal medium at concentrations ranging from about 70% to about 100%, from about 75% to about 100%, from about 80% to about 95% or more, from about 80% to about 95% or more, or from about 85% to about 95% or more. In some aspects, the second culture medium comprises αMEM at a concentration ranging from about 85% to about 95%. In some aspects, the second culture medium comprises αMEM at a concentration of about 90%.

A second medium of the instant disclosure also comprises a serum or serum substitute. Serums or serum substitutes are known by individuals of skill in the art and can be as described in Section I(b) herein above. In some aspects, a second culture medium of the instant disclosure comprises an animal-derived serum. In some aspects, a second culture medium of the instant disclosure comprises a serum substitute. In some aspects, a second culture medium of the instant disclosure comprises a serum or serum substitute selected from KSR, B27 supplement, N2 supplement, SR1, SR2, StemPro Serum Replacement, ITS or ITS-X supplement, E8 supplement, TeSR™-E8™, mTeSR™1, chemically defined lipid concentrate, or any combination thereof. A nutrient rich second culture medium of the instant disclosure can comprise a nutrient-rich basal medium at concentrations ranging from about 1% to about 20%, from about 5% to about 15%, or from about 8% to about 12%. In some aspects, a second culture medium of the instant disclosure comprises KSR serum substitute. In some aspects, the second culture medium comprises KSR at a concentration ranging from about 8% to about 12%. In some aspects, the second culture medium comprises KSR at a concentration of about 10%.

A second culture medium of the instant disclosure also comprises a gonadotropin. Gonadotropins are a class of glycoprotein hormones that are secreted by the pituitary gland (or, in some cases, the placenta) and act on the gonads (testes or ovaries) to regulate reproductive processes, including gametogenesis, steroidogenesis, and sexual maturation. Gonadotropins function by binding to specific receptors on target cells in the gonads, thereby stimulating the production and maturation of germ cells and the synthesis of sex steroids. Non-limiting examples of gonadotropins include follicle-stimulating hormone (FSH), luteinizing hormone (LH), human chorionic gonadotropin (hCG), and equine chorionic gonadotropin (eCG, also known as pregnant mare serum gonadotropin or PMSG). In some aspects, the gonadotropin is follicle-stimulating hormone (FSH), luteinizing hormone (LH), chorionic gonadotropin (CG), or any combination thereof. In some aspects, the gonadotropin is FSH. For instance, a second culture medium of the instant disclosure can comprise FSH at concentrations ranging from about 1 ng/ml to about 200 ng/ml, from about 10 ng/ml to about 50 ng/mL, from about 10 ng/mL to about 25 ng/ml, from about 100 ng/ml to about 200 ng/ml, or from about 15 ng/mL to about 25 ng/ml. In some aspects, the second culture medium comprises FSH at a concentration ranging from about 15 ng/ml to about 25 ng/ml. In some aspects, the second culture medium comprises FSH at a concentration of about 20 ng/mL.

A second cell culture medium of the instant disclosure further comprises an androgen. As used herein, the term “androgen” refers to any natural or synthetic steroid hormone that binds to and activates the androgen receptor, thereby regulating the development, maintenance, and function of male reproductive tissues and secondary sexual characteristics. Androgens play a critical role in spermatogenesis, promoting the proliferation and differentiation of germ cells and supporting the function of somatic cells within the testis. In some aspects, the second cell culture medium comprises a high concentration of an androgen. Non-limiting examples of androgens include testosterone, dihydrotestosterone (DHT), androstenedione, dehydroepiandrosterone (DHEA), methyltestosterone, and fluoxymesterone.

In some aspects, the androgen is testosterone, dihydrotestosterone (DHT), androstenedione, dehydroepiandrosterone (DHEA), methyltestosterone, fluoxymesterone, or any combination thereof. In some aspects, the androgen is testosterone. Testosterone can be used in a cell culture medium at a concentration ranging from about 0.1 uM to about 20 uM, from about 5 uM to about 15 uM, of from about 9 uM to about 11 uM. In some aspects, the second culture medium comprises testosterone at a concentration ranging from about 9 uM to about 11 uM. In some aspects, the second culture medium comprises testosterone at a concentration of about 10 uM.

A second culture medium of the instant disclosure also comprises a pituitary extract or a supplement comprising pituitary-derived hormones and growth factors. A pituitary extract or a supplement comprising pituitary-derived hormones and growth factors is a preparation derived from the pituitary gland of an animal or human source, or a composition containing purified, recombinant, or synthetic hormones and growth factors that are normally produced by the pituitary gland. These preparations provide a complex mixture of biologically active proteins, peptides, and other factors that support cell growth, proliferation, differentiation, and function in vitro. Non-limiting examples of pituitary extracts include bovine pituitary extract (BPE), porcine pituitary extract, ovine pituitary extract, human pituitary extract, and equine pituitary extract. Supplements comprising pituitary-derived hormones and growth factors may include purified or recombinant forms of follicle-stimulating hormone (FSH), luteinizing hormone (LH), growth hormone (GH), prolactin, adrenocorticotropic hormone (ACTH), thyroid-stimulating hormone (TSH), and other pituitary hormones, either alone or in combination.

In some aspects, the pituitary extract or a supplement comprising pituitary-derived hormones and growth factors is bovine pituitary extract (BPE), porcine pituitary extract, ovine pituitary extract, human pituitary extract, equine pituitary extract, a supplement comprising purified or recombinant pituitary hormones and growth factors, or any combination thereof. In some aspects, the pituitary extract or a supplement comprising pituitary-derived hormones and growth factors is BPE. BPE can be used in cell culture at concentrations ranging from about 10 ug/mL to about 100 ug/mL, from about 20 ug/mL to about 80 ug/mL, from about 30 ug/mL to about 70 ug/mL, or from about 40 ug/mL to about 60 ug/mL. In some aspects, the second culture medium comprises BPE at a concentration ranging from about 40 ug/mL to about 60 ug/mL. In some aspects, the second culture medium comprises BPE at a concentration of about 50 ug/mL.

The second cell culture medium also comprises a conditionally essential amino acid. As used herein, a “conditionally essential amino acid” refers to an amino acid that is normally synthesized by the body but may become essential under certain physiological or pathological conditions, such as rapid cell growth, cellular stress, or in vitro culture, where endogenous synthesis may be insufficient to meet cellular demands. In cell culture, these amino acids can be supplemented to support optimal cell growth, viability, and function. Non-limiting examples of conditionally essential amino acids used in media include glutamine, arginine, cysteine, tyrosine, proline, glycine, serine, ornithine, and histidine, as well as stabilized forms or derivatives thereof. In some aspects, the conditionally essential amino acid is glutamine, arginine, cysteine, tyrosine, proline, glycine, serine, ornithine, histidine, stabilized forms thereof, or combinations thereof. In some aspects, the conditionally essential amino acid is L-Glutamine or a stabilized form thereof. In some aspects, the second culture medium comprises L-Glutamine, a L-alanyl-L-glutamine dipeptide, or a combination thereof. In some aspects, the second culture medium comprises L-Glutamine or a L-alanyl-L-glutamine dipeptide at a concentration ranging from about 0.5 mM to about 5 mM.

The second culture medium can optionally further comprise an agonist of a c-KIT receptor. As used herein, an “agonist of a c-KIT receptor” refers to any molecule, peptide, protein, or compound that binds to and activates the c-KIT receptor (also known as CD117), thereby stimulating downstream signaling pathways involved in cell survival, proliferation, and differentiation, particularly in germ cells and hematopoietic cells. Non-limiting examples of c-KIT receptor agonists include stem cell factor (SCF; also known as KIT ligand or KITLG), recombinant human stem cell factor, recombinant mouse stem cell factor, granulocyte-macrophage colony-stimulating factor (GM-CSF), FLT3 ligand, and interleukin-3 (IL-3), as well as any functionally equivalent peptide, protein, small molecule agonist of the c-KIT receptor, or any combination thereof.

In some aspects, the agonist of a c-KIT receptor is KIT ligand (KITLG; also known as stem cell factor (SCF)), granulocyte-macrophage colony-stimulating factor (GM-CSF), FLT3 ligand, or interleukin-3 (IL-3). In some aspects, the agonist of a c-KIT receptor is KITLG. KITLG can be used in cell culture at concentrations ranging from about 0.1 ng/ml to about 100 ng/ml, from about 0.1 ng/ml to about 90 ng/ml, from about 0.1 ng/mL to about 80 ng/ml, from about 0.1 ng/ml to about 70 ng/ml, from about 0.1 ng/ml to about 60 ng/ml, from about 0.1 ng/mL to about 50 ng/ml, from about 0.1 ng/ml to about 40 ng/ml, from about 0.1 ng/ml to about 30 ng/ml, from about 0.1 ng/ml to about 20 ng/ml, from about 0.1 ng/ml to about 10 ng/ml, from about 0.1 ng/ml to about 5 ng/mL, from about 0.5 ng/mL to about 100 ng/ml, from about 5 ng/ml to about 50 ng/ml, or from about 0.5 ng/ml to about 1.5 ng/ml. In some aspects, the second culture medium comprises KITLG at a concentration ranging from about 0.5 ng/ml to about 1.5 ng/ml. In some aspects, the second culture medium comprises KITLG at a concentration of about 1 ng/ml.

The second cell culture medium can further comprise one or more additional common cell culture ingredients selected from the group consisting of a diluent or buffered salt solution such as Earle's Balanced Salt Solution (EBSS), Hanks' Balanced Salt Solution (HBSS), or phosphate-buffered saline (PBS); an antibiotic or antimicrobial agent such as penicillin-streptomycin (Pen/Strep), gentamicin, or amphotericin B; a pH buffer such as HEPES or sodium bicarbonate; and other standard cell culture supplements. In some aspects, the second cell culture medium further comprises Pen/Strep. In some aspects, the second cell culture medium further comprises Pen/Strep at a concentration ranging from bout 0.5% to about 1.5%. the second cell culture medium further comprises Pen/Strep at a concentration of about 1%.

The second culture medium of the instant disclosure can be used to culture human spermatocytes arrested before the pachytene stage of meiotic prophase I for a second period of time. The second period of time can be sufficient to allow human spermatocytes arrested before the pachytene stage of meiotic prophase I to progress through completion of meiosis and formation of round spermatids. As described in the examples herein below, the inventors tried a number of culture periods to identify culture periods sufficient to allow the cells to progress through, and complete meiosis. The inventors discovered that the second culture period can range from about 5 days to about 15 days or longer. For instance, the second culture period can be about 5 days, about 6 days, about 7 days, about 8 days, about 9 days, about 10 days, about 11 days, about 12 days, about 13 days, about 14 days or about 15 days, or longer. In some aspects, the second culture period ranges from about 5 days to about 15 days. In some aspects, the second culture period ranges from about 8 days to about 12 days. In some aspects, the second culture period is about 5 days. In some aspects, the second culture period is about 10 days.

Completion of meiosis and formation of round spermatids in the second culture medium is evidenced by the appearance of cells expressing one or more round spermatid-specific markers after culturing the spermatocytes in the second culture medium for the second culture period. Methods for determining the developmental stage of a germ cell, including progression through meiosis and the identification of round spermatids, are well known to those skilled in the art. In general, the presence of round spermatids can be determined by the appearance of cells characterized by the expression of spermatid-specific markers such as ACRV1, TNP1, PRM1, CCDC185, DNAJB7, or any combination thereof, and optionally by the loss or reduction of markers characteristic of diploid or tetraploid meiotic cells, such as SYCP3 and SYCP1. In some aspects, the appearance of round spermatids is further evidenced by the appearance of cells expressing ACRV1. The transition from pachytene spermatocytes to round spermatids may also be accompanied by the reduction, absence, or relocalization of meiotic prophase markers such as HORMAD1 and γH2AX from autosomes, or by the restriction of γH2AX to the sex body, and by the disappearance of DMC1 and RAD51 foci, indicating completion of meiotic divisions and the formation of haploid cells.

In some aspects, completion of meiosis and formation of round spermatids further comprises progression of human spermatocytes arrested before the pachytene stage of meiotic prophase I through the pachytene stage as evidenced by the appearance of cells characterized by the presence, absence, or localization of a cell marker indicative of spermatocytes at or beyond the pachytene stage selected from HORMAD1, γH2AX, SYCP1, SYCP3, DMC1, RAD51, MEIOB, REC8, PRDM9, STRA8, or any combination thereof. In some aspects, completion of meiosis and formation of round spermatids further comprises progression of human spermatocytes arrested before the pachytene stage of meiotic prophase I through the pachytene stage as evidenced by the appearance of cells characterized by the presence, absence, or localization of HORMAD1 and γH2AX from autosomes, or by the restriction of γH2AX to the sex body, and optionally by the absence of cells expressing ACRV1.

### B. Feeder Cells

In cell culture systems, support materials are often used to provide a microenvironment that promotes cell attachment, survival, proliferation, and differentiation. Common support systems include extracellular matrix components such as Matrigel, Geltrex, or laminin, as well as synthetic or natural substrates that mimic the in vivo cellular niche. These materials can facilitate cell adhesion and provide biochemical cues that influence cell behavior. In addition to matrix-based supports, co-culture systems with other cell types, such as feeder layers, are frequently employed to supply essential growth factors, extracellular matrix proteins, and cell-cell interactions that are critical for the maintenance and development of specialized cell populations.

Importantly, according to the invention described herein, only feeder cells were effective as a support system for promoting the progression and completion of meiosis and the formation of round spermatids in vitro. Other support materials, such as Matrigel or Geltrex, were tested but did not enable successful meiotic progression or spermatid formation. Accordingly, a second culture system of the instant disclosure further comprises feeder cells.

Feeder cells, as used in the context of this invention, refer to a layer of living or mitotically inactivated cells that are co-cultured with the target germ cells to provide trophic support, secrete growth factors, and help maintain an appropriate microenvironment. Feeder cells can be derived from a variety of sources and may include mouse embryonic fibroblasts (MEFs), human embryonic fibroblasts, human foreskin fibroblasts, STO cells, SNL cells, or any combination thereof. In some aspects, the feeder cells are MEFs. These feeder cells can be used alone or in combination and are typically mitotically inactivated to prevent overgrowth while still providing the necessary support for germ cell development.

In some aspects, the feeder cells are MEFs. In some aspects, the feeder cells are MEFs

### C. Aspects of Second Culture Systems

In some aspects, a second cell culture system for supporting the progression of human spermatocytes arrested before the pachytene stage of meiotic prophase I through completion of meiosis and formation of round spermatids in vitro comprises: a second cell culture medium for culture of human spermatocytes arrested before the pachytene stage of meiotic prophase I for about 10 days, the third culture medium comprising αMEM at a concentration ranging from about 80% to about 95%; gonadotropin at a concentration ranging from about 15 ng/mL to about 25 ng/ml; testosterone at a high concentration ranging from about 9 uM to about 11 uM; BPE at a concentration ranging from about 40 ug/mL to about 60 ug/mL; L-Glutamine or a L-alanyl-L-glutamine dipeptide at a concentration ranging from about 0.5 mM to about 5 mM; and optionally KITLG at a concentration ranging from about 0.5 ng/ml to about 1.5 ng/mL.

The second culture system also comprises MEF feeder cells. The appearance of cells expressing one or more round spermatid-specific markers is evidenced by the appearance of cells expressing ACRV1.

### (d) Spermiogenesis

An additional aspect of the instant disclosure encompasses a third cell culture system for the generation of human spermatozoa from round spermatids in vitro.

The round spermatids used for in vitro culture and differentiation to spermatozoa in the third culture system can be isolated directly from human testicular tissue, which may be obtained from a living or cadaveric donor using methods recognized by those skilled in the art. Alternatively, the round spermatids can be derived from cells previously cultured in vitro, including those generated through earlier stages of in vitro spermatogenesis. In some aspects, the round spermatids are freshly isolated, while in other aspects, the round spermatids can be obtained from cryopreserved or previously frozen testicular tissue or cell preparations. The invention is compatible with a wide range of cell sources for spermatid culture, including cells from prepubertal or adult individuals, fertile or infertile subjects, and tissue or cells that have been stored for future use, such as for fertility preservation prior to gonadotoxic treatments.

In some aspects, the round spermatids used for in vitro spermiogenesis are round spermatids isolated from testicular tissue. In other aspects, the round spermatids are obtained from cells previously cultured in vitro. In some aspects, the round spermatids are obtained from cells previously cultured in vitro as described in Section I(c) herein above, using methods described in Section III herein below. In some aspects, the round spermatids are obtained from cells previously cultured in vitro as described by sequential differentiation of SSCs or dSPGs under the culture conditions described herein above in Sections I(b) and I(c).

The third culture system comprises a third cell culture medium for culturing round spermatids for a third culture period. The third culture medium comprises a nutrient-rich basal medium at a sufficiently high concentration to provide a nutrient rich medium. A nutrient-rich medium can be as described in Section I(c) A. In some aspects, the nutrient-rich basal medium of a second culture medium of the instant disclosure is Minimum Essential Medium Alpha (αMEM), Advanced Protein-free Eukaryotic cell Line medium 2 (APEL2), Medium 199 (M199), Dulbecco's Modified Eagle Medium/Nutrient Mixture F-12 (DMEM/F12), or any combination thereof. In some aspects, the nutrient-rich basal medium is αMEM.

A nutrient rich third culture medium of the instant disclosure can comprise a nutrient-rich basal medium at concentrations ranging from about 70% to about 100%, from about 75% to about 100%, from about 80% to about 95% or more, from about 80% to about 95% or more, or from about 85% to about 95% or more. In some aspects, the third culture medium comprises αMEM at a concentration ranging from about 85% to about 95%. In some aspects, the third culture medium comprises αMEM at a concentration of about 90%.

A third medium of the instant disclosure also comprises a serum or serum substitute. Serums or serum substitutes are known by individuals of skill in the art and can be as described in Section I(b) herein above. In some aspects, a third culture medium of the instant disclosure comprises an animal-derived serum. In some aspects, a third culture medium of the instant disclosure comprises a serum substitute. In some aspects, a third culture medium of the instant disclosure comprises a serum or serum substitute selected from KSR, B27 supplement, N2 supplement, SR1, SR2, StemPro Serum Replacement, ITS or ITS-X supplement, E8 supplement, TeSR™-E8TM, mTeSR™1, chemically defined lipid concentrate, or any combination thereof. A nutrient rich third culture medium of the instant disclosure can comprise a nutrient-rich basal medium at concentrations ranging from about 1% to about 20%, from about 5% to about 15%, or from about 8% to about 12%. In some aspects, a third culture medium of the instant disclosure comprises KSR serum substitute. In some aspects, the third culture medium comprises KSR at a concentration ranging from about 8% to about 12%. In some aspects, the third culture medium comprises KSR at a concentration of about 10%.

The third culture medium also comprises a reducing agent or antioxidant. As used herein, a “reducing agent or antioxidant” refers to a compound that helps maintain a reduced (non-oxidizing) environment in cell culture media by neutralizing reactive oxygen species (ROS) and preventing oxidative damage to cellular components. Reducing agents and antioxidants are commonly added to culture media to protect cells from oxidative stress, enhance cell viability, and support cellular processes that are sensitive to redox balance, such as differentiation and maturation. Non-limiting examples of reducing agents and antioxidants used in cell culture include 2-mercaptoethanol (β-mercaptoethanol), dithiothreitol (DTT), cysteine, glutathione, N-acetylcysteine, ascorbic acid (vitamin C), and any combination thereof.

In some aspects, the reducing agent or antioxidant is 2-mercaptoethanol, dithiothreitol (DTT), cysteine, glutathione, N-acetylcysteine, ascorbic acid, or any combination thereof. In some aspects, the reducing agent or antioxidant is 2-mercaptoethanol. 2-mercaptoethanol can be used in culture media at concentrations ranging from about 25 uM to about 100 uM or concentrations between 40 uM and 55 uM. the third culture medium comprises 2-mercaptoethanol at a concentration ranging from about 45 uM to about 55 uM.

A third cell culture medium of the instant disclosure further comprises an androgen. An adnreogen can be as described in Section I(c) A.

In some aspects, the androgen is testosterone, dihydrotestosterone (DHT), androstenedione, dehydroepiandrosterone (DHEA), methyltestosterone, fluoxymesterone, or any combination thereof. In some aspects, the androgen is testosterone. Testosterone can be used in a cell culture medium at a concentration ranging from about 0.1 uM to about 20 uM, from about 5 uM to about 15 uM, of from about 9 uM to about 11 uM. In some aspects, the third culture medium comprises testosterone at a concentration ranging from about 3 uM to about 7 uM. In some aspects, the second culture medium comprises testosterone at a concentration of about 5 uM.

A third culture medium of the instant disclosure also comprises a retinoid or retinoic acid receptor agonist. Retinoid or retinoic acid receptor agonists can be as described in Section I(b). In some aspects, the retinoid or retinoic acid receptor agonist is all-trans retinoic acid, 9-cis retinoic acid, 13-cis retinoic acid, retinol, retinal, retinyl acetate, retinyl palmitate, AM580, TTNPB, adapalene, tazarotene, bexarotene, acitretin, or any combination thereof. In some aspects, the retinoid or retinoic acid receptor agonist is retinoic acid. In some aspects, the third culture medium comprises retinoic acid at a concentration ranging from about 4 uM to about 6 uM.

The third culture medium comprises conditioned media, a metabolizable monocarboxylate compound, or both. In some aspects, the third culture medium comprises conditioned media. In some aspects, the third culture medium comprises a metabolizable monocarboxylate compound. In some aspects, the third culture medium comprises conditioned media and a metabolizable monocarboxylate compound.

A third culture medium of the instant disclosure can also comprise conditioned media. Conditioned cell culture media are known to individuals of skill in the art. A conditioned cell culture medium is a cell culture medium that has been previously incubated with one or more cell types, allowing the medium to accumulate secreted factors, extracellular matrix components, metabolites, and other bioactive molecules produced by those cells. Conditioned media can be used to provide a supportive environment for the growth, survival, differentiation, or maturation of target cells by supplying trophic factors and signaling molecules that can mimic aspects of the in vivo cellular niche.

Methods of preparing conditioned media is known to individuals of skill in the art. In short, a base culture medium is incubated with a selected cell type such as mouse embryonic fibroblasts (MEFs), human embryonic fibroblasts, or other feeder cells, for a defined period that can be 24 to 72 hours. After incubation, the medium can be collected, centrifuged or filtered to remove cells and debris, and can be used directly or diluted with fresh medium before being applied to the target cell culture. In some aspects, the conditioned media comprises culture medium that has been previously incubated with one or more cell types selected from the group consisting of mouse embryonic fibroblasts (MEFs), human embryonic fibroblasts, human foreskin fibroblasts, STO cells, SNL cells, or any functionally equivalent feeder cell, such that the medium contains secreted factors, extracellular matrix components, or metabolites produced by said cells. In some aspects, the conditioned media comprises MEF-conditioned culture medium. In some aspects, the conditioned media comprises MEF-conditioned third culture medium. In some aspects, the third culture medium comprises MEF-conditioned third culture medium at a rate of one part MEF-conditioned third cell culture medium to three parts third medium.

As used herein, a “metabolizable monocarboxylate compound” refers to an organic molecule containing a single carboxyl group (—COOH) that can be taken up and metabolized by cells as an energy source or metabolic substrate. In cell culture media, metabolizable monocarboxylate compounds can be added to support cellular metabolism, provide alternative energy sources, and promote specific differentiation or maturation processes, such as spermiogenesis. Non-limiting examples of metabolizable monocarboxylate compounds include lactate, pyruvate, acetate, and β-hydroxybutyrate.

In some aspects, the metabolizable monocarboxylate compound is lactate, pyruvate, acetate, β-hydroxybutyrate, or any combination thereof. In some aspects, the metabolizable monocarboxylate compound is lactate. Lactate can be used in cell culture media at concentrations ranging from about 0.5 mM to about 2 mM or from about 0.8 mM to about 1.2 mM. In some aspects, the third culture medium optionally comprises lactate at a concentration ranging from about 0.8 mM to about 1.2 mM.

### (e) Complete Spermatogenesis Culture System

Yet another aspect of the instant disclosure encompasses a cell culture system for the generation of human spermatozoa from dSPGs or SSCs in vitro through the sequential culture of SSCs or dSPGs. The in vitro culture system induces entry of human SSCs or dSPGs into meiosis, supports the progression of human spermatocytes arrested before the pachytene stage of meiotic prophase I through completion of meiosis and formation of round spermatids produced in the preceding culture system, and subsequently enables the generation of human spermatozoa from the round spermatids produced in the preceding culture system.

The system comprises a first culture system for inducing entry of dSPGs or SSCs into meiosis and generating spermatocytes arrested before the pachytene stage of meiosis I. The first culture system comprises a first culture medium for culturing human dSPGs or SSCs for a first culture period. The first culture medium comprises a nutrient-rich basal medium and a serum or serum substitute, wherein the nutrient-rich basal medium is present at a sufficiently low concentration to provide a nutrient-restricted environment, a retinoid or retinoic acid receptor agonist, a TGF-β superfamily ligand, insulin, and a WNT pathway regulator. The first culture system can be as described in Section I(b) herein above.

The system further comprises a second culture system for supporting the progression of human spermatocytes arrested before the pachytene stage of meiotic prophase I through completion of meiosis and formation of round spermatids. The second culture system comprises a second culture medium for culturing human spermatocytes for a second culture period. The second culture medium comprises a nutrient-rich basal medium and a serum or serum substitute, wherein the nutrient-rich basal medium is present at a sufficiently high concentration to provide a nutrient-rich environment. The second culture medium further comprises a gonadotropin, an androgen, a pituitary extract or a supplement comprising pituitary-derived hormones and growth factors, a conditionally essential amino acid, optionally an agonist of a c-KIT receptor, and feeder cells. The second culture system can be as described in Section I(c) herein above.

The system further comprises a third culture system for the generation of human elongated spermatids from round spermatids. The third culture system comprises a third culture medium for culturing round spermatids for a third culture period. The third culture medium comprises a nutrient-rich basal medium and a serum or serum substitute, wherein the nutrient-rich basal medium is present at a sufficiently high concentration to provide a nutrient-rich environment. The third cell culture medium further comprises a reducing agent or antioxidant, a high concentration of an androgen, a retinoid or retinoic acid receptor agonist, and conditioned media, a metabolizable monocarboxylate compound, or both. The third culture system can be as described in Section I(d) herein above.

Entry of human dSPGs or SSCs into meiosis is evidenced by the appearance of spermatocytes arrested before the pachytene stage of meiosis I, as characterized by the presence, absence, or localization of cell markers indicative of cells before the pachytene stage after culturing the dSPGs or SSCs in the cell culture system during the first and second culture periods. Completion of meiosis and formation of round spermatids is evidenced by the appearance of cells expressing one or more round spermatid-specific markers. Generation of human spermatozoa from round spermatids is evidenced by the appearance of flagellar cells.

In some aspects, the culture system comprises: a first cell culture system for entry of human dSPGs and SSCs into meiosis; a second cell culture system for supporting the progression of human spermatocytes arrested before the pachytene stage of meiotic prophase I through completion of meiosis and formation of round spermatids; and a third cell culture system for generation of human spermatozoa from round spermatids.

The first culture system comprises a first cell culture medium for culturing human dSPGs and SSCs for about 4 days. The first cell culture medium comprises EBSS at a concentration of about 90% (v/v); αMEM at a concentration of about 10% (v/v); KSR at a concentration of about 1% (v/v); Pen-Strep at a concentration of about 1%; BMP2, BMP4, and BMP7 each at a concentration of about 20 ng/ml; retinoic acid at a concentration of about 5 uM; activin A at a concentration of about 100 ng/ml; insulin at a concentration of about 50 ug/mL; and IWR-endo-1 at a concentration of about 3 uM.

The second cell culture system comprises a second cell culture medium for culture of human spermatocytes arrested before the pachytene stage of meiotic prophase and MEF feeder cells. The second culture system comprises αMEM at a concentration ranging from about 80% to about 95%; gonadotropin at a concentration ranging from about 15 ng/ml to about 25 ng/ml; testosterone at a high concentration ranging from about 9 uM to about 11 uM; BPE at a concentration ranging from about 40 ug/mL to about 60 ug/mL; L-Glutamine or a L-alanyl-L-glutamine dipeptide at a concentration ranging from about 0.5 mM to about 5 mM; and optionally KITLG at a concentration ranging from about 0.5 ng/ml to about 1.5 ng/mL.

The third culture system comprises a third cell culture medium for culture of round spermatids for a third culture period, the third culture medium comprising αMEM at a concentration ranging from about 80% to about 95%; KSR at a concentration ranging from about 8% to about 12%; 2-mercaptoethanol at a concentration ranging from about 45 uM to about 55 uM; testosterone at a concentration ranging from about 3 uM to about 7 uM; retinoic acid at a concentration ranging from about 4 uM to about 6 uM; and one part MEF-conditioned third cell culture medium to three parts third culture medium, lactate at a concentration ranging from about 0.8 mM to about 1.2 mM, or both.

Entry of the human dSPGs and SSCs into meiosis is evidenced by the appearance of spermatocytes arrested before the pachytene stage of meiosis I after culturing the dSPGs and SSCs in the cell culture system during the first culture period, wherein the spermatocytes are characterized by the presence of HORMAD1 on unsynapsed chromosome axes and presence of γH2AX diffusely throughout the nucleus, prior to its restriction to the sex body; absence, reduction, or relocalization of HORMAD1 and γH2AX from autosomes; or any combination thereof; wherein the appearance of cells expressing one or more round spermatid-specific markers is evidenced by the appearance of cells expressing ACRV1; and wherein generation of human spermatozoa from round spermatids is evidenced by the appearance of flagellar cells.

### II. Cell Cultures

One aspect of the instant disclosure encompasses sequential and stage-specific cell cultures that enable the complete in vitro differentiation of human dSPGs and SSCs through multiple defined steps, including induction of meiotic entry, progression through meiotic prophase and completion of meiosis to form haploid round spermatids, and subsequent spermiogenesis resulting in the production of elongated, flagellated spermatids, then spermatids.

### (f) Meiotic Entry

An additional aspect of the instant disclosure encompasses a first cell culture for inducing entry of human dSPGs or SSCs into meiosis in vitro. The first cell culture comprises isolated human dSPGs or SSCs and a first culture system comprising a first cell culture medium for culturing human dSPGs or SSCs for a first culture period. The human dSPGs, human SSCs, and first culture system can be as described in Section I(b). In some aspects, the first cell culture further comprises human spermatocytes arrested before the pachytene stage of meiotic prophase I.

### (g) Meiotic Progression and Completion

Yet another aspect of the instant disclosure encompasses a second cell culture for supporting the progression of human spermatocytes arrested before the pachytene stage of meiotic prophase I through completion of meiosis and formation of round spermatids in vitro. The second cell culture comprises isolated human pre-pachytene spermatocytes and a second culture system comprising a second cell culture medium and feeder cells for culturing the human pre-pachytene spermatocytes for a second culture period. The human pre-pachytene spermatocytes and second culture system can be as described in Section I(c). In some aspects, the second cell culture further comprises human round spermatids.

### (h) Spermiogenesis

One aspect of the instant disclosure encompasses a third cell culture for the generation of human spermatozoa from round spermatids in vitro. The third cell culture comprises a third culture system comprising a third cell culture medium for culturing round spermatids for a third culture period. The round spermatids and the third culture system can be as described in Section I(d). In some aspects, the third cell culture further comprises human flagellar spermatocytes.

### III. Method of In Vitro Culture

One aspect of the instant disclosure encompasses a method of generating human spermatozoa from differentiating spermatogonia (dSPGs) or spermatogonial stem cells (SSCs) in vitro. As described herein and in the examples, the inventors developed culture systems that guide the development of cells through the successive stages of spermatogenesis. Accordingly, the method comprises sequentially culturing dSPGs or SSCs in three distinct culture systems, each specifically optimized to support a different stage of spermatogenesis, such that the cells are guided through meiotic entry, meiotic progression and completion, and spermiogenesis, ultimately resulting in the formation of spermatozoa.

Importantly, in addition to identifying appropriate culture systems and media for cell development, the inventors discovered that an optimal temperature for growing these cells is 32° C., which is lower than the 35° C. typically used by individuals of skill in the art for culturing testicular germ cells.

Methods of the instant disclosure comprise using a first culture system for culturing human dSPGs or SSCs in a first culture medium for a first culture period sufficient for entry of human the dSPGs or SSCs into meiosis as evidenced by the appearance of spermatocytes arrested before the pachytene stage of meiosis I, as characterized by the presence, absence, or localization of cell markers indicative of cells before the pachytene stage, after culturing the dSPGs and SSCs in the first culture medium for the first culture period. A first culture system can be as described in Section I(b).

The methods further comprise transferring the cells resulting from the first culture period to a second culture medium of a second culture system, and culturing the cells for a second culture period sufficient for the progression of human spermatocytes arrested before the pachytene stage of meiotic prophase I through completion of meiosis and formation of round spermatids as evidenced by the appearance of cells expressing one or more round spermatid-specific markers. A second culture system can be as described in Section I(c).

According to methods of the instant disclosure, methods of the instant disclosure further comprise transferring the cells resulting from the second culture period to a third culture medium of a third culture system, and culturing for a third culture period sufficient for generation of human spermatozoa from round spermatids as evidenced by the appearance of flagellar cells. A third culture system can be as described in Section I(d).

In some aspects, the cells are cultured in the three culture systems at a temperature ranging from about 30° C. to about 34° C., from about 31° C. to about 33° C., or at a temperature of about 32° C. In some aspects, the cells are cultured at a temperature of about 32° C.

Another aspect of the instant disclosure encompasses a method of inducing or promoting entry of human differentiating spermatogonia (dSPGs) and spermatogonial stem cells (SSCs) into meiosis in vitro. The method comprises using a first culture system to culture dSPGs or SSCs in a first culture system for a first culture period sufficient for the progression of human spermatocytes arrested before the pachytene stage of meiotic prophase I through completion of meiosis and formation of round spermatids as evidenced by the appearance of cells expressing one or more round spermatid-specific markers. A first culture system can be as described in Section I(b). In some aspects, the cells are cultured in the three culture systems at a temperature ranging from about 30° C. to about 34° C., from about 31° C. to about 33° C., or at a temperature of about 32° C. In some aspects, the cells are cultured at a temperature of about 32° C.

An additional aspect of the instant disclosure encompasses inducing or promoting progression of human spermatocytes arrested before the pachytene stage of meiotic prophase I through completion of meiosis and formation of round spermatids in vitro. The method comprises using a second culture system to culture the pre-pachytene spermatids for a second culture period sufficient for the progression of human spermatocytes arrested before the pachytene stage of meiotic prophase I through completion of meiosis and formation of round spermatids as evidenced by the appearance of cells expressing one or more round spermatid-specific markers. A second culture system can be as described in Section I(c). In some aspects, the cells are cultured in the three culture systems at a temperature ranging from about 30° C. to about 34° C., from about 31° C. to about 33° C., or at a temperature of about 32° C. In some aspects, the cells are cultured at a temperature of about 32° C.

Yet another aspect of the instant disclosure encompasses a method for generation of human spermatozoa from round spermatids in vitro. The method comprises using a third culture system to culture the round spermatids for a third culture period sufficient for the generation of spermatozoa as evidenced by the appearance of the spermatozoa. A third culture system can be as described in Section I(d). In some aspects, the cells are cultured in the three culture systems at a temperature ranging from about 30° C. to about 34° C., from about 31° C. to about 33° C., or at a temperature of about 32° C. In some aspects, the cells are cultured at a temperature of about 32° C.

One aspect of the instant disclosure encompasses a method of generating human spermatozoa from differentiating spermatogonia (dSPGs) or spermatogonial stem cells (SSCs) in vitro. The method comprises culturing human dSPGs or SSCs in a first culture medium of a first culture system; transferring the cells resulting from culturing in the first culture medium to a second culture medium of a second culture system and culturing the cells using the second culture system; followed by transferring the cells resulting from culturing in the second culture system to a third culture medium of a third culture system and culturing the cells in the third culture system to obtain spermatozoa.

The first culture medium comprises a nutrient-rich basal medium and a serum or serum substitute, wherein the nutrient-rich basal medium is present at a sufficiently low concentration to provide a nutrient-restricted medium, wherein the sufficiently low concentration of nutrient-rich basal medium ranges from about 8% to about 12% of the total medium volume and the balance comprising a diluent or buffered salt solution at a concentration ranging from about 88% to about 92%; retinoic acid at a concentration ranging from about 4 uM to about 6 uM; BMP2, BMP4 and BMP7 each at a concentration ranging from about 15 ng/mL to about 25 ng/ml; activin A at a concentration ranging from about 90 ng/ml to about 110 ng/ML; insulin at a concentration ranging from about 40 ug/mL to about 60 ug/mL; and IWR-endo-1 at a concentration ranging from about 2 uM to about 4 uM.

The dSPGs and SSCs are cultured using the first culture medium at a temperature ranging from about 31° C. to about 33° C. for a first culture period sufficient for entry of the human dSPGs or SSCs into meiosis. In some aspects, the first culture period is about 1 day to about 4 days.

Entry of the human dSPGs and SSCs into meiosis is evidenced by EdU incorporation indicating DNA replication and the appearance of spermatocytes arrested before the pachytene stage of meiosis I as characterized by the presence, absence, or localization of HORMAD1, γH2AX, DMC1, RAD51, STRA8, or any combination thereof. In some aspects, entry of the human dSPGs and SSCs into meiosis can be further evidenced by the appearance of spermatocytes arrested before the pachytene stage of meiosis I characterized by EdU incorporation indicating DNA replication; the presence of HORMAD1 on unsynapsed chromosome axes and presence of γH2AX diffusely throughout the nucleus, prior to its restriction to the sex body; absence, reduction, or relocalization of HORMAD1 and γH2AX from autosomes; or any combination thereof.

The second culture system comprises a second culture medium and MEF feeder cells. The second culture medium comprises a nutrient-rich basal medium and a serum or serum substitute, wherein the nutrient-rich basal medium is present at a sufficiently high concentration to provide a nutrient-rich medium, wherein the sufficiently high concentration of nutrient-rich basal medium ranges from about 85% to about 95% of the total medium volume; FSH at a concentration ranging from about 15 ng/ml to about 25 ng/ml; testosterone at a concentration ranging from about 9 uM to about 11 uM; bovine pituitary extract (BPE) at a concentration ranging from about 40 ug/mL to about 60 ug/mL; L-Glutamine or a L-alanyl-L-glutamine dipeptide at a concentration ranging from about 0.5 mM to about 5 mM; and optionally c-KIT receptor ligand (KITLG) at a concentration ranging from about 0.5 ng/ml to about 1.5 ng/ml.

The cells resulting from culturing in the first culture medium are cultured at a temperature ranging from about 31° C. to about 33° C. for a second culture period sufficient for the progression of the human spermatocytes arrested before the pachytene stage of meiotic prophase I through completion of meiosis and formation of round spermatids. In some aspects, the second culture period ranges from about 8 days to about 12 days.

Progression of the human spermatocytes arrested before the pachytene stage of meiotic prophase I through completion of meiosis and formation of round spermatids is evidenced b by the appearance of cells expressing ACRV1.

The third culture medium comprises a nutrient-rich basal medium and a serum or serum substitute, wherein the nutrient-rich basal medium is present at a sufficiently high concentration to provide a nutrient-rich medium, wherein the sufficiently high concentration of nutrient-rich basal medium ranges from about 85% to about 95% of the total medium volume; 2-mercaptoethanol at a concentration ranging from about 45 uM to about 55 uM; testosterone at a concentration ranging from about 3 uM to about 7 uM; retinoic acid at a concentration ranging from about 4 uM to about 6 uM; and conditioned media comprising MEF-conditioned third culture medium, lactate at a concentration ranging from about 0.8 mM to about 1.2 mM, or a combination thereof. In some aspects, the third culture medium comprises MEF-conditioned third culture medium at a rate of one part MEF-conditioned third cell culture medium to three parts third culture medium.

The third culture system is used to culture the cells in the third culture system at a temperature ranging from about 31° C. to about 33° C. for a third culture period sufficient for generation of human spermatozoa from the round spermatids. Generation of human spermatozoa from the round spermatids is evidenced by the appearance of flagellar cells. In some aspects, the third culture period is about 5 to about 8 days.

In some aspects, the first culture medium comprises αMEM basal medium at a concentration ranging from about 8% to about 12%. In some aspects, the first culture medium comprises KSR serum substitute at a concentration ranging from about 0.1% to about 2%. In some aspects, the first culture medium comprises αMEM basal medium at a concentration ranging from about 8% to about 12%, EBSS at a concentration ranging from about 88% to about 92%, KSR serum substitute at a concentration ranging from about 0.1% to about 2%, BMP2, BMP4, and BMP7 each at a concentration ranging from about 15 ng/ml to about 25 ng/ml, retinoic acid at a concentration ranging from about 4 uM to about 6 uM, activin A at a concentration ranging from about 90 ng/ml to about 110 ng/ml, insulin at a concentration ranging from about 40 ug/mL to about 60 ug/mL, and IWR-endo-1 at a concentration ranging from about 2 uM to about 4 uM.

### IV. Kits

A further aspect of the present disclosure encompasses a kit for entry of human differentiating spermatogonia (dSPGs) and spermatogonial stem cells (SSCs) into meiosis in vitro; a kit for supporting the progression of human spermatocytes arrested before the pachytene stage of meiotic prophase I through completion of meiosis and formation of round spermatids in vitro; a kit for generation of human spermatozoa from round spermatids in vitro; or any combination thereof.

A kit for entry of human differentiating spermatogonia (dSPGs) and spermatogonial stem cells (SSCs) into meiosis in vitro comprises a first culture system. The first culture system can be as described in Section I(b).

A kit for supporting the progression of human spermatocytes arrested before the pachytene stage of meiotic prophase I through completion of meiosis and formation of round spermatids in vitro comprises a second culture system. The second culture system can be as described in Section I(c).

A kit for generation of human spermatozoa from round spermatids in vitro comprises a third culture system. The third culture system can be as described in Section I(d).

Another aspect of the instant disclosure encompasses a kit for generation of human falgellated spermatids from dSPGs and SSCs in vitro. The kit comprises a first culture system for entry of dSPGs and SSCs into meiosis to generate spermatocytes arrested before the pachytene stage of meiosis I, a second culture system for supporting the progression of human spermatocytes arrested before the pachytene stage of meiotic prophase I through completion of meiosis and formation of round spermatids, and a third cell culture system for generation of human elongated spermatids from the round spermatids. The first culture system can be as described in Section (b). The second culture system can be as described in Section I(c). The third culture system can be as described in Section I(d).

Kits according to the present disclosure can include one or more additional reagents useful for culturing testicular tissue and germ cells according to the present disclosure. A kit generally includes a package with one or more containers holding the reagents, as one or more separate compositions or, optionally, as admixture where the compatibility of the reagents will allow. The test kit can also include other material(s), which may be desirable from a user standpoint, such as a buffer(s), a diluent(s), a standard(s), and/or any other material useful in processing or conducting any other step of the tagging method.

Kits according to the present disclosure preferably include instructions for culturing testicular tissue and germ cells. Instructions included in kits of the present disclosure can be affixed to packaging material or can be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

### V. Aspects

1. A first cell culture system for entry of human differentiating spermatogonia (dSPGs) and spermatogonial stem cells (SSCs) into meiosis in vitro, the first culture system comprising a first cell culture medium for culture of human dSPGs and SSCs for a first culture period, the first culture medium comprising:
   a. a nutrient-rich basal medium and a serum or serum substitute, wherein the nutrient-rich basal medium is at a sufficiently low concentration to provide a nutrient restriction medium;
   b. a retinoid or retinoic acid receptor agonist;
   C. a TGF-β superfamily ligand;
   d. insulin; and
   e. a WNT pathway regulator; and
   wherein entry of human dSPGs and SSCs into meiosis is evidenced by the appearance of spermatocytes arrested before the pachytene stage of meiosis I, as characterized by the presence, absence, or localization of cell markers indicative of cells before the pachytene stage, after culturing the dSPGs and SSCs in the first culture medium for the first culture period.
2. The first culture system of claim 1, wherein the WNT pathway regulator is IWR-endo-1, DKK1, XAV939, ICG-001, C59, LGK974, PRI-724, PNU-74654, FH535, Wnt-C59, DKK2, sFRP1, or any combination thereof.
3. The culture first system of claim 1 or claim 2, wherein the WNT pathway regulator is a WNT pathway antagonist selected from IWR-endo-1, DKK1, XAV939, ICG-001, C59, LGK974, PRI-724, PNU-74654, FH535, Wnt-C59, DKK2, sFRP1, or any combination thereof.
4. The first culture system of any one of the preceding claims, wherein the WNT pathway regulator is IWR-endo-1.
5. The first culture system of any one of the preceding claims, wherein the first culture medium comprises IWR-endo-1 at a concentration ranging from about 2 uM to about 4 uM.
6. The first culture system of any of the preceding claims, wherein the insulin in the first culture medium is human insulin, porcine insulin, bovine insulin, insulin-like growth factor 1 (IGF-1), insulin-like growth factor 2 (IGF-2), or any combination thereof.
7. The first culture system of any of the preceding claims, wherein the insulin in the first culture medium is human insulin.
8. The first culture system of any one of the preceding claims, wherein the first culture medium comprises insulin at a concentration ranging from about 40 ug/mL to about 60 ug/mL.
9. The first culture system of any one of the preceding claims, wherein the nutrient-rich basal medium is Minimum Essential Medium Alpha (αMEM), Advanced Protein-free Eukaryotic cell Line medium 2 (APEL2), Medium 199 (M199), Dulbecco's Modified Eagle Medium/Nutrient Mixture F-12 (DMEM/F12), or any combination thereof.
10. The first culture system of any one of the preceding claims, wherein the nutrient-rich basal medium is αMEM.
11. The first culture system of any one of the preceding claims, wherein the first culture medium comprises αMEM at a concentration ranging from about 8% to about 12%.
12. The first culture system of any one of the preceding claims, wherein the serum or serum substitute is Knockout Serum Replacement (KSR), B27 supplement, N2 supplement, Serum Replacement 1 (SR1), Serum Replacement 2 (SR2), StemPro Serum Replacement, ITS or ITS-X supplement, Essential 8 (E8) supplement, TeSR™-E8™ mTeSR™1, chemically defined lipid concentrate, or any combination thereof.
13. The first culture system of any one of the preceding claims, wherein the serum or serum substitute is KSR.
14. The first culture system of any one of the preceding claims, wherein the first culture medium comprises KSR at a concentration ranging from about 0.1% to about 2%.
15. The first culture system of any one of the preceding claims, wherein the retinoid or retinoic acid receptor agonist is all-trans retinoic acid, 9-cis retinoic acid, 13-cis retinoic acid, retinol, retinal, retinyl acetate, retinyl palmitate, AM580, TTNPB, adapalene, tazarotene, bexarotene, acitretin, or any combination thereof.
16. The first culture system of any one of the preceding claims, wherein the retinoid or retinoic acid receptor agonist is retinoic acid.
17. The first culture system of any one of the preceding claims, wherein the first culture medium comprises retinoic acid at a concentration ranging from about 4 uM to about 6 uM.
18. The first culture system of any one of the preceding claims, wherein the TGF-β superfamily ligand is activin A, activin B, activin AB, activin C, activin E, transforming growth factor beta 1 (TGF-(1), transforming growth factor beta 2 (TGF-β2), transforming growth factor beta 3 (TGF-(3), bone morphogenetic proteins (BMPs), growth differentiation factor 5 (GDF5), growth differentiation factor 6 (GDF6), growth differentiation factor 7 (GDF7), growth differentiation factor 9 (GDF9), growth differentiation factor 11 (GDF11), nodal, myostatin (GDF8), or any combination thereof.
19. The first culture system of any one of the preceding claims, wherein the TGF-β superfamily ligand is a BMP and an activin.
20. The first culture system of claim 19, wherein the BMP is BMP1, BMP2, BMP3, BMP4, BMP5, BMP6, BMP7, BMP8a, BMP8b, BMP9, BMP10, BMP11, BMP12, BMP13, BMP14, BMP15, or any combination thereof.
21. The first culture system of claim 20, wherein the BMP is BMP2, BMP4 and BMP7.
22. The first culture system of claim 19, wherein the activin is activin A, activin B, activin AB, activin C, activin E, or any combination thereof.
23. The first culture system of claim 22, wherein the activin is activin A.
24. The first culture system of claim 23, wherein the first culture medium comprises activin A at a concentration ranging from about 90 ng/ml to about 110 ng/ml.
25. The first culture system of claim 19, wherein the first culture medium comprises BMP2, BMP4 and BMP7 each at a concentration ranging from about 15 ng/ml to about 25 ng/ml and activin A at a concentration ranging from about 90 ng/ml to about 110 ng/mL.
26. The first culture system of any one of the preceding claims, wherein the first culture medium comprises αMEM, KSR, BMP2, BMP4, BMP7, retinoic acid, activin A, insulin, and IWR-endo-1.
27. The first culture system of any one of the preceding claims, wherein the first culture medium comprises αMEM at a concentration ranging from about 8% to about 12%, KSR at a concentration ranging from about 0.1% to about 2%, BMP2 at a concentration ranging from about 15 ng/ml to about 25 ng/ml, BMP4 at a concentration ranging from about 15 ng/ml to about 25 ng/ml, BMP7 at a concentration ranging from about 15 ng/ml to about 25 ng/ml, retinoic acid at a concentration ranging from about 4 uM to about 6 uM, activin A at a concentration ranging from about 90 ng/mL to about 110 ng/ml, insulin at a concentration ranging from about 40 ug/mL to about 60 ug/mL, and IWR-endo-1 at a concentration ranging from about 2 uM to about 4 uM.
28. The first culture system of any one of the preceding claims, wherein the first cell culture medium further comprises one or more additional common cell culture ingredients selected from the group consisting of a diluent or buffered salt solution such as Earle's Balanced Salt Solution (EBSS), Hanks' Balanced Salt Solution (HBSS), or phosphate-buffered saline (PBS); an antibiotic or antimicrobial agent such as penicillin-streptomycin (Pen/Strep), gentamicin, or amphotericin B; a pH buffer such as HEPES or sodium bicarbonate; and other standard cell culture supplements.
29. The first culture system of any one of the preceding claims, wherein the first cell culture medium further comprises EBSS.
30. The first culture system of any one of the preceding claims, wherein the first cell culture medium further comprises EBSS at a concentration ranging from about 80% to about 95.
31. The first culture system of any one of the preceding claims, wherein the first cell culture medium further comprises Pen/Strep.
32. The first culture system of any one of the preceding claims, wherein the first cell culture medium further comprises Pen/Strep at a concentration ranging from bout 0.5% to about 1.5%.
33. The first culture system of any one of the preceding claims, wherein the first cell culture medium further comprises EBSS and Pen/Strep.
34. The first culture system of any one of the preceding claims, wherein the first cell culture medium further comprises EBSS at a concentration ranging from about 80% to about 95%, and Pen/Strep at a concentration ranging from bout 0.5% to about 1.5%.
35. The first cell culture system of any one of the preceding claims, wherein the first culture period ranges from about 12 hrs to about 5 days.
36. The first cell culture system of any one of the preceding claims, wherein the first culture period is about 1 day to about 4 days.
37. The first cell culture system of any one of the preceding claims, wherein entry of the human dSPGs and SSCs into meiosis is evidenced by DNA replication and the appearance of spermatocytes arrested before the pachytene stage of meiosis I characterized by the presence, absence, or localization of HORMAD1, γH2AX, DMC1, RAD51, STRA8, or any combination thereof.
38. The first cell culture system of any one of the preceding claims, wherein entry of the human dSPGs and SSCs into meiosis is evidenced by the appearance of spermatocytes arrested before the pachytene stage of meiosis I characterized by EdU incorporation indicating DNA replication; the presence of HORMAD1 on unsynapsed chromosome axes; presence of γH2AX diffusely throughout the nucleus, prior to its restriction to the sex body; presence of SYCP1 as punctate or partial linear staining, prior to full synapsis; presence of DMC1 and/or RAD51 as nuclear foci, with the number of foci decreasing as cells progress to pachytene; presence of MEIOB, REC8, PRDM9, or STRA8, with STRA8 expression being downregulated as cells progress through prophase I; absence, reduction, or relocalization of HORMAD1 and γH2AX from autosomes, or disappearance of DMC1 and RAD51 foci, indicating progression beyond the pre-pachytene stage; or any combination thereof.
39. The first cell culture system of any one of the preceding claims, wherein entry of the human dSPGs and SSCs into meiosis is evidenced by the appearance of spermatocytes arrested before the pachytene stage of meiosis I characterized by EdU incorporation indicating DNA replication; the presence of HORMAD1 on unsynapsed chromosome axes and presence of γH2AX diffusely throughout the nucleus, prior to its restriction to the sex body; absence, reduction, or relocalization of HORMAD1 and YH2AX from autosomes; or any combination thereof.
40. A first cell culture system for entry of human dSPGs and SSCs into meiosis in vitro, the culture system comprising a first cell culture medium for culturing human dSPGs and SSCs for about 4 days, the first cell culture medium comprising:
   a. EBSS at a concentration of about 90% (v/v);
   b. αMEM at a concentration of about 10% (v/v);
   c. KSR at a concentration of about 1% (v/v);
   d. Pen-Strep at a concentration of about 1%;
   e. BMP2, BMP4, and BMP7 each at a concentration of about 20 ng/ml;
   f. retinoic acid at a concentration of about 5 uM;
   g. activin A at a concentration of about 100 ng/ml;
   h. insulin at a concentration of about 50 ug/mL; and
   i. IWR-endo-1 at a concentration of about 3 uM;
   wherein entry of the human dSPGs and SSCs into meiosis is evidenced by the appearance of spermatocytes arrested before the pachytene stage of meiosis I after culturing the dSPGs and SSCs in the cell culture system during the first culture period, wherein the spermatocytes are characterized by the presence of HORMAD1 on unsynapsed chromosome axes and presence of γH2AX diffusely throughout the nucleus, prior to its restriction to the sex body; absence, reduction, or relocalization of HORMAD1 and γH2AX from autosomes; or any combination thereof.
41. A second cell culture system for supporting the progression of human spermatocytes arrested before the pachytene stage of meiotic prophase I through completion of meiosis and formation of round spermatids in vitro, the second culture system comprising:
   a. a second cell culture medium for culture of human spermatocytes arrested before the pachytene stage of meiotic prophase I for a second culture period, the second culture medium comprising:
      i. a nutrient-rich basal medium and a serum or serum substitute, wherein the nutrient-rich basal medium is at a sufficiently high concentration to provide a nutrient rich medium;
      ii. a gonadotropin;
      iii. an androgen;
      iv. a pituitary extract or a supplement comprising pituitary-derived hormones and growth factors;
      v. a conditionally essential amino acid; and
      vi. optionally an agonist of a c-KIT (CD117) receptor; and
   b. feeder cells;
   wherein completion of meiosis and formation of round spermatids is evidenced by the appearance of cells expressing one or more round spermatid-specific markers.
42. The second culture system of claim 41, wherein the nutrient-rich basal medium is Minimum Essential Medium Alpha (αMEM), Advanced Protein-free Eukaryotic cell Line medium 2 (APEL2), Medium 199 (M199), Dulbecco's Modified Eagle Medium/Nutrient Mixture F-12 (DMEM/F12), or any combination thereof.
43. The second culture system of claim 41 or claim 42, wherein the nutrient-rich basal medium is αMEM.
44. The second culture system of any one of claims 41-43, wherein the second culture medium comprises αMEM at a concentration ranging from about 85% to about 95%.
45. The second culture system of any one of claims 41-44, wherein the serum or serum substitute is Knockout Serum Replacement (KSR), B27 supplement, N2 supplement, Serum Replacement 1 (SR1), Serum Replacement 2 (SR2), StemPro Serum Replacement, ITS or ITS-X supplement, Essential 8 (E8) supplement, TeSR™-E8™, mTeSR™1, chemically defined lipid concentrate, or any combination thereof.
46. The second culture system of any one of claims 41-45, wherein the serum or serum substitute is KSR.
47. The second culture system of any one of claims 41-46, wherein the second culture medium comprises KSR at a concentration ranging from about 8% to about 12%.
48. The second culture system of any one of claims 41-47, wherein the gonadotropin is follicle-stimulating hormone (FSH), luteinizing hormone (LH), chorionic gonadotropin (CG), or any combination thereof.
49. The second culture system of any one of claims 41-48, wherein the gonadotropin is FSH.
50. The second culture system of any one of claims 41-49, wherein the second culture medium comprises FSH at a concentration ranging from about 15 ng/mL to about 25 ng/mL.
51. The second culture system of any one of claims 41-50, wherein the androgen is testosterone, dihydrotestosterone (DHT), or any combination thereof.
52. The second culture system of any one of claims 41-51, wherein the androgen is testosterone.
53. The second culture system of any one of claims 41-52, wherein the second culture medium comprises testosterone at a concentration ranging from about 9 uM to about 11 uM 54. The second culture system of any one of claims 41-53, wherein the pituitary extract or a supplement comprising pituitary-derived hormones and growth factors is bovine pituitary extract (BPE), porcine pituitary extract, ovine pituitary extract, human pituitary extract, equine pituitary extract, a supplement comprising purified or recombinant pituitary hormones and growth factors, or any combination thereof.
55. The second culture system of any one of claims 41-54, wherein the pituitary extract or a supplement comprising pituitary-derived hormones and growth factors is BPE.
56. The second culture system of any one of claims 41-55, wherein the second culture medium comprises BPE at a concentration ranging from about 40 ug/mL to about 60 ug/mL.
57. The second culture medium of any of claims 41-56, wherein the second culture medium comprises αMEM, KSR, gonadotropin, testosterone at a high concentration, and BPE.
58. The second culture system of any one of claims 41-57, wherein the second culture medium comprises αMEM at a concentration ranging from about 80% to about 95%, KSR at a concentration ranging from about 8% to about 12%, gonadotropin at a concentration ranging from about 15 ng/ml to about 25 ng/ml, testosterone at a high concentration ranging from about 9 uM to about 11 uM, and BPE at a concentration ranging from about 40 ug/mL to about 60 ug/mL.
59. The second culture system of any one of claims 41-58, wherein the conditionally essential amino acid is glutamine, arginine, cysteine, tyrosine, proline, glycine, serine, ornithine, histidine, stabilized forms thereof, or combinations thereof.
60. The second culture system of any one of claims 41-59, wherein the conditionally essential amino acid is L-Glutamine or a stabilized form thereof.
61. The second culture system of any one of claims 41-60, wherein the second culture medium comprises L-Glutamine, a L-alanyl-L-glutamine dipeptide, or a combination thereof.
62. The second culture system of any one of claims 41-61, wherein the second culture medium comprises L-Glutamine or a L-alanyl-L-glutamine dipeptide at a concentration ranging from about 0.5 mM to about 5 mM.
63. The second culture system of any one of claims 41-62, wherein the agonist of a c-KIT receptor is KIT ligand (KITLG; also known as stem cell factor (SCF)), granulocyte-macrophage colony-stimulating factor (GM-CSF), FLT3 ligand, or interleukin-3 (IL-3).
64. The second culture system of any one of claims 41-63, wherein the agonist of a c-KIT receptor is KITLG.
65. The culture system of any one of claims 41-64, wherein the second culture medium comprises KITLG at a concentration ranging from about 0.5 ng/ml to about 1.5 ng/mL.
66. The culture system of any one of claims 41-65, wherein the feeder cells are mouse embryonic fibroblasts (MEFs), human embryonic fibroblasts, human foreskin fibroblasts, STO cells, SNL cells, or any combination thereof.
67. The culture system of any one of claims 41-66, wherein the feeder cells are MEFs.
68. The culture system of any one of claims 41-67, wherein the second culture period ranges from about 5 days to about 15 days.
69. The culture system of any one of claims 41-68, wherein the second cell culture medium further comprises one or more additional common cell culture ingredients selected from the group consisting of a diluent or buffered salt solution such as Earle's Balanced Salt Solution (EBSS), Hanks' Balanced Salt Solution (HBSS), or phosphate-buffered saline (PBS); an antibiotic or antimicrobial agent such as penicillin-streptomycin (Pen/Strep), gentamicin, or amphotericin B; a pH buffer such as HEPES or sodium bicarbonate; and other standard cell culture supplements.
70. The culture system of any one of claims 41-69, wherein the second cell culture medium further comprises Pen/Strep.
71. The culture system of any one of claims 41-70, wherein the second cell culture medium further comprises Pen/Strep at a concentration ranging from about 0.5% to about 1.5%.
72. The culture system of any one of claims 41-71, wherein the appearance of cells expressing one or more round spermatid-specific markers is evidenced by the appearance of cells expressing one or more spermatid-specific markers selected from the group consisting of ACRV1, TNP1, PRM1, CCDC185, DNAJB7, and any combination thereof, and optionally the loss or reduction of one or more markers characteristic of diploid or tetraploid meiotic cells selected from the group consisting of SYCP3, SYCP1, and any combination thereof.
73. The cell culture system of any one of claims 41-72, wherein the appearance of cells expressing one or more round spermatid-specific markers is evidenced by the appearance of cells expressing ACRV1.
74. The culture system of any one of claims 41-73, wherein completion of meiosis and formation of round spermatids further comprises progression of human spermatocytes arrested before the pachytene stage of meiotic prophase I through the pachytene stage as evidenced by the appearance of cells characterized by the presence, absence, or localization of a cell marker indicative of spermatocytes beyond the pachytene stage selected from HORMAD1, γH2AX, SYCP1, SYCP3, DMC1, RAD51, MEIOB, REC8, PRDM9, STRA8, or any combination thereof.
75. The culture system of any one of claims 41-74, wherein completion of meiosis and formation of round spermatids further comprises progression of human spermatocytes arrested before the pachytene stage of meiotic prophase I through the pachytene stage as evidenced by the appearance of cells characterized by the presence, absence, or localization of HORMAD1 and γH2AX from autosomes, or by the restriction of γH2AX to the sex body, and optionally by the absence of cells expressing ACRV1.
76. A second cell culture system for supporting the progression of human spermatocytes arrested before the pachytene stage of meiotic prophase I through completion of meiosis and formation of round spermatids in vitro, the second culture system comprising:
   a. a second cell culture medium for culture of human spermatocytes arrested before the pachytene stage of meiotic prophase I for about 10 days, the third culture medium comprising αMEM at a concentration ranging from about 80% to about 95%; gonadotropin at a concentration ranging from about 15 ng/ml to about 25 ng/ml; testosterone at a high concentration ranging from about 9 uM to about 11 uM; BPE at a concentration ranging from about 40 ug/mL to about 60 ug/mL; L-Glutamine or a L-alanyl-L-glutamine dipeptide at a concentration ranging from about 0.5 mM to about 5 mM; and optionally KITLG at a concentration ranging from about 0.5 ng/ml to about 1.5 ng/ml; and
   b. MEF feeder cells;
   wherein the appearance of cells expressing one or more round spermatid-specific markers is evidenced by the appearance of cells expressing ACRV1.
77. A third cell culture system for generation of human spermatozoa from round spermatids in vitro, the culture system comprising: a third cell culture medium for culture of round spermatids for a third culture period, the third culture medium comprising:
   i. a nutrient-rich basal medium and a serum or serum substitute, wherein the nutrient-rich basal medium is at a sufficiently high concentration to provide a nutrient rich medium;
   ii. a reducing agent or antioxidant;
   iii. an androgen;
   iv. a retinoid or retinoic acid receptor agonist; and
   v. conditioned media, a metabolizable monocarboxylate compound, or both;
   wherein generation of human spermatozoa from round spermatids is evidenced by the appearance of spermatozoa.
78. The third culture system of claim 77, wherein the nutrient-rich basal medium is Minimum Essential Medium Alpha (αMEM), Advanced Protein-free Eukaryotic cell Line medium 2 (APEL2), Medium 199 (M199), Dulbecco's Modified Eagle Medium/Nutrient Mixture F-12 (DMEM/F12), or any combination thereof.
79. The third culture system of claim 77 or claim 78, wherein the nutrient-rich basal medium is αMEM.
80. The third culture system of any one of claims 77-79, wherein the third culture medium comprises αMEM at a concentration ranging from about 80% to about 95%.
81. The third culture system of any one of claims 77-80, wherein the serum or serum substitute is Knockout Serum Replacement (KSR), B27 supplement, N2 supplement, Serum Replacement 1 (SR1), Serum Replacement 2 (SR2), StemPro Serum Replacement, ITS or ITS-X supplement, Essential 8 (E8) supplement, TeSR™-E8™ mTeSR™1, chemically defined lipid concentrate, or any combination thereof.
82. The third culture system of any one of claims 77-81, wherein the serum or serum substitute is KSR.
83. The third culture system of any one of claims 77-82, wherein the third culture medium comprises KSR at a concentration ranging from about 8% to about 12%.
84. The third culture system of any one of claims 77-83, wherein the reducing agent or antioxidant is 2-mercaptoethanol, dithiothreitol (DTT), cysteine, glutathione, N-acetylcysteine, ascorbic acid, or any combination thereof.
85. The third culture system of any one of claims 77-84, wherein the reducing agent or antioxidant is 2-mercaptoethanol.
86. The third culture system of any one of claims 77-85, wherein the third culture medium comprises 2-mercaptoethanol at a concentration ranging from about 45 uM to about 55 uM.
87. The third culture system of any one of claims 77-86, wherein the androgen is testosterone, dihydrotestosterone (DHT), or any combination thereof.
88. The third culture system of any one of claims 77-87, wherein the androgen is testosterone.
89. The third culture system of any one of claims 77-88, wherein the third culture medium comprises testosterone at a concentration ranging from about 3 uM to about 7 uM.
90. The third culture system of any one of claims 77-89, wherein the retinoid or retinoic acid receptor agonist is all-trans retinoic acid, 9-cis retinoic acid, 13-cis retinoic acid, retinol, retinal, retinyl acetate, retinyl palmitate, AM580, TTNPB, adapalene, tazarotene, bexarotene, acitretin, or any combination thereof.
91. The third culture system of any one of claims 77-90, wherein the retinoid or retinoic acid receptor agonist is retinoic acid.
92. The third culture system of any one of claims 77-91, wherein the first culture medium comprises retinoic acid at a concentration ranging from about 4 uM to about 6 uM.
93. The third culture system of any one of claims 77-92, wherein the conditioned media comprises culture medium that has been previously incubated with one or more cell types selected from the group consisting of mouse embryonic fibroblasts (MEFs), human embryonic fibroblasts, human foreskin fibroblasts, STO cells, SNL cells, or any functionally equivalent feeder cell, such that the medium contains secreted factors, extracellular matrix components, or metabolites produced by said cells.
94. The third culture system of any one of claims 77-93, wherein the conditioned media comprises MEF-conditioned culture medium.
95. The third culture system of any one of claims 77-94, wherein the conditioned media comprises MEF-conditioned third culture medium.
96. The third culture system of any one of claims 77-95, wherein the third culture medium comprises MEF-conditioned third culture medium at a rate of one part MEF-conditioned third cell culture medium to three parts third medium.
97. The third culture system of any one of claims 77-96, wherein the metabolizable monocarboxylate compound is lactate, pyruvate, acetate, β-hydroxybutyrate, or any combination thereof.
98. The third culture system of any one of claims 77-97, wherein the metabolizable monocarboxylate compound is lactate.
99. The third culture system of any one of claims 77-98, wherein the third culture medium optionally comprises lactate at a concentration ranging from about 0.8 mM to about 1.2 mM.
100. The third culture system of any one of claims 77-99, wherein the third culture medium comprises αMEM, KSR, 2-mercaptoethanol, testosterone, retinoic acid, and MEF-conditioned third culture medium or lactate.
101. The third culture system of any one of claims 77-100, wherein the third culture medium comprises αMEM at a concentration ranging from about 80% to about 95%; KSR at a concentration ranging from about 8% to about 12%.;-mercaptoethanol at a concentration ranging from about 45 uM to about 55 uM; testosterone at a high concentration ranging from about 9 uM to about 11 uM; retinoic acid at a concentration ranging from about 4 uM to about 6 uM; and MEF-conditioned third culture medium at a rate of one part MEF-conditioned medium to three parts third medium or lactate at a concentration ranging from about 0.8 mM to about 1.2 mM.
102. The c third ell culture system of any one of claims 77-101, wherein the third culture period ranges from about 3 days to about 10 days.
103. The third cell culture system of any one of claims 77-102, wherein the third culture period is about 5 to about 8 days.
104. A third cell culture system for generation of human spermatozoa from round spermatids in vitro, the culture system comprising:
   a. a third cell culture medium for culture of round spermatids for a third culture period, the third culture medium comprising:
      i. αMEM at a concentration ranging from about 80% to about 95%;
      ii. KSR at a concentration ranging from about 8% to about 12%;
      iii. 2-mercaptoethanol at a concentration ranging from about 45 uM to about 55 uM;
      iv. testosterone at a concentration ranging from about 3 uM to about 7 uM;
      v. retinoic acid at a concentration ranging from about 4 uM to about 6 uM; and
      vi. one part MEF-conditioned third cell culture medium to three parts third culture medium, lactate at a concentration ranging from about 0.8 mM to about 1.2 mM, or both;
   wherein generation of human spermatozoa from round spermatids is evidenced by the appearance of spermatozoa.
105. A cell culture system for generation of human falgellated spermatids from dSPGs and SSCs in vitro, the culture system comprising:
   a. a first culture system for entry of dSPGs and SSCs into meiosis, the first cell culture system comprising a first culture medium for culture of human dSPGs and SSCs for a first culture period, the first culture medium comprising:
      i. a nutrient-rich basal medium and a serum or serum substitute, wherein the nutrient-rich basal medium is at a sufficiently low concentration to provide a nutrient restriction first medium;
      ii. a retinoid or retinoic acid receptor agonist;
      iii. a TGF-β superfamily ligand;
      iv. insulin; and
      v. a WNT pathway regulator;
   b. a second cell culture system for supporting the progression of human spermatocytes arrested before the pachytene stage of meiotic prophase I through completion of meiosis and formation of round spermatids, the second culture system comprising:
      i. a second medium for culture of human spermatocytes arrested before the pachytene stage of meiotic prophase I for a second culture period, the second culture medium comprising:
         1) a nutrient-rich basal medium and a serum or serum substitute, wherein the nutrient-rich basal medium is at a sufficiently high concentration to provide a nutrient rich second medium;
         2) a gonadotropin;
         3) a high concentration of an androgen;
         4) a pituitary extract or a supplement comprising pituitary-derived hormones and growth factors;
         5) a conditionally essential amino acid; and
         6) optionally an agonist of a c-KIT receptor; and
      ii. feeder cells;
   c. a third cell culture system for generation of human elongated spermatids from round spermatids, the third cell culture system comprising a third cell culture medium for culture of round spermatids for a third culture period, the third culture medium comprising:
      i. a nutrient-rich basal medium and a serum or serum substitute, wherein the nutrient-rich basal medium is at a sufficiently high concentration to provide a nutrient rich third medium;
      ii. a reducing agent or antioxidant;
      iii. a high concentration of an androgen;
      iv. a retinoid or retinoic acid receptor agonist; and
      v. conditioned media, a metabolizable monocarboxylate compound, or both;
   wherein entry of human dSPGs and SSCs into meiosis is evidenced by the appearance of spermatocytes arrested before the pachytene stage of meiosis I, wherein the spermatocytes are characterized by the presence, absence, or localization of cell markers indicative of cells before the pachytene stage of meiosis I after culturing the dSPGs and SSCs in the cell culture system during the first and second culture periods; wherein completion of meiosis and formation of round spermatids is evidenced by the appearance of cells expressing one or more round spermatid-specific markers; and wherein generation of human spermatozoa from round spermatids is evidenced by the appearance of flagellar cells.
106. A cell culture system for generation of human falgellated spermatids from dSPGs or SSCs in vitro, the culture system comprising:
   a. a first cell culture system for entry of human dSPGs and SSCs into meiosis in vitro, the culture system comprising a first cell culture medium for culturing human dSPGs and SSCs for about 4 days, the first cell culture medium comprising: EBSS at a concentration of about 90% (v/v); αMEM at a concentration of about 10% (v/v); KSR at a concentration of about 1% (v/v); Pen-Strep at a concentration of about 1%; BMP2, BMP4, and BMP7 each at a concentration of about 20 ng/ml; retinoic acid at a concentration of about 5 μM; activin A at a concentration of about 100 ng/ml; insulin at a concentration of about 50 g/mL; and IWR-endo-1 at a concentration of about 3 μM
   b. a second cell culture system for supporting the progression of human spermatocytes arrested before the pachytene stage of meiotic prophase I through completion of meiosis and formation of round spermatids in vitro, the second culture system comprising:
      i. a second cell culture medium for culture of human spermatocytes arrested before the pachytene stage of meiotic prophase I for about 10 days, the third culture medium comprising αMEM at a concentration ranging from about 80% to about 95%; gonadotropin at a concentration ranging from about 15 ng/ml to about 25 ng/mL; testosterone at a high concentration ranging from about 9 uM to about 11 uM; BPE at a concentration ranging from about 40 ug/mL to about 60 ug/mL; L-Glutamine or a L-alanyl-L-glutamine dipeptide at a concentration ranging from about 0.5 mM to about 5 mM; and optionally KITLG at a concentration ranging from about 0.5 ng/ml to about 1.5 ng/ml;
         1) and
      ii. MEF feeder cells;
   c. a third cell culture system for generation of human spermatozoa from round spermatids in vitro, the culture system comprising:
      i. a third cell culture medium for culture of round spermatids for a third culture period, the third culture medium comprising αMEM at a concentration ranging from about 80% to about 95%; KSR at a concentration ranging from about 8% to about 12%; 2-mercaptoethanol at a concentration ranging from about 45 uM to about 55 uM; testosterone at a concentration ranging from about 3 uM to about 7 uM; retinoic acid at a concentration ranging from about 4 uM to about 6 uM; and one part MEF-conditioned third cell culture medium to three parts third culture medium, lactate at a concentration ranging from about 0.8 mM to about 1.2 mM, or both;
   wherein entry of the human dSPGs and SSCs into meiosis is evidenced by the appearance of spermatocytes arrested before the pachytene stage of meiosis I after culturing the dSPGs and SSCs in the cell culture system during the first culture period, wherein the spermatocytes are characterized by the presence of HORMAD1 on unsynapsed chromosome axes and presence of γH2AX diffusely throughout the nucleus, prior to its restriction to the sex body; absence, reduction, or relocalization of HORMAD1 and γH2AX from autosomes; or any combination thereof; wherein the appearance of cells expressing one or more round spermatid-specific markers is evidenced by the appearance of cells expressing ACRV1; and wherein generation of human spermatozoa from round spermatids is evidenced by the appearance of spermatozoa.
107. A method of generating human spermatozoa from differentiating spermatogonia (dSPGs) or spermatogonial stem cells (SSCs) in vitro, the method comprising:
   a. culturing human dSPGs or SSCs in a first culture medium of a first culture system of any one of claims 1-x for a first culture period sufficient for entry of human the dSPGs or SSCs into meiosis as evidenced by the appearance of spermatocytes arrested before the pachytene stage of meiosis I, as characterized by the presence, absence, or localization of cell markers indicative of cells before the pachytene stage, after culturing the dSPGs and SSCs in the first culture medium for the first culture period;
   b. transferring the cells resulting from step (a) to a second culture medium of a second culture system of any one of claims x to x, and culturing for a second culture period sufficient for the progression of human spermatocytes arrested before the pachytene stage of meiotic prophase I through completion of meiosis and formation of round spermatids as evidenced by the appearance of cells expressing one or more round spermatid-specific markers;
   c. transferring the cells resulting from step (b) to a third culture medium of a third culture system of any one of claims x to x, and culturing for a third culture period sufficient for generation of human spermatozoa from round spermatids as evidenced by the appearance of flagellar cells.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred aspects(s) thereof, the articles “a”, “an”, “the” and “said” are intended to mean that there are one or more of the elements. The terms “comprising”, “including” and “having” are intended to be inclusive and mean that there may be additional elements other than the listed elements.

As used herein, the term “about” is intended to provide a range of values that are considered equivalent to the stated value as understood by one of ordinary skill in the art. In general, “about” modifies a stated value to encompass variations that are within ±20%, within ±10%, within ±5%, or within ±1% of the stated value, unless otherwise specified or dictated by the context.

As used herein, the term “culture system” refers to any in vitro setup configured to support the survival, maintenance, proliferation, or differentiation of cells. A culture system comprises at least a culture medium and may further include one or more of the following: feeder cells, extracellular matrix components, scaffolds (e.g., hydrogels or solid supports), culture vessels (e.g., tissue culture plates, flasks, bioreactors, or microfluidic devices), and equipment that regulates environmental conditions such as temperature, humidity, gas composition (e.g., CO₂, O₂), or mechanical stimulation. The term encompasses both static and dynamic systems, including but not limited to two-dimensional and three-dimensional cultures, perfusion systems, rotating wall vessels, and automated or semi-automated cell culture platforms.

“Mammalian,” as used herein refers to both human subjects (and cells sources) and non-human subjects (and cell sources or types), such as dog, cat, mouse, monkey, etc. (e.g., for veterinary purposes).

As used herein, the term “spermatogonia” refers to any germ cells before meiosis II, including diploid or tetraploid testicular germ cell, such as SSCs differentiating SPGs, and spermatocytes.

As used herein, the term “spermatids” refers to any haploid testicular germ cell resulting from meiosis II and subsequent differentiation, including round spermatids and spermatozoa.

The terms “medium”, “media”, “culture medium”, or “culture media,” as used herein, refers to an aqueous based solution that is provided for the growth, viability, or storage of cells used in carrying out the present invention.

A “base media,” as used herein, refers to a basal salt nutrient or an aqueous solution of salts and other elements that provide cells with water and certain bulk inorganic ions essential for normal cell metabolism and maintains intra-cellular and/or extra-cellular osmotic balance. Base media can comprise energy sources such as glucose or galactose, amino acids, vitamins, salts, a buffering system to maintain the medium within the physiological pH range, or any combination thereof. Base media for culturing mammalian cells are known in the art and can be available commercially. Non-limiting examples of base media include phosphate buffered saline (PBS), Dulbecco's Modified Eagle's Medium (DMEM), Minimal Essential Medium (MEM), Basal Medium Eagle (BME), Roswell Park Memorial Institute Medium (RPMI) 1640, MCDB 131, Click's medium, McCoy's 5A Medium, Medium 199, William's Medium E, insect media such as Grace's medium, Ham's Nutrient mixture F-10 (Ham's F-10), Ham's F-12, a-Minimal Essential Medium (αMEM), Glasgow's Minimal Essential Medium (G-MEM) and Iscove's Modified Dulbecco's Medium.

In the context of testicular cells, certain specialized media have been developed. For instance, F12/DMEM, often supplemented with fetal bovine serum (FBS), is commonly used. Other media, like M199, can also be utilized. In addition, specific supplements may be included depending on the cell type. For instance, for the culture of Sertoli cells (a type of testicular cell), supplements can include insulin, transferrin, and biotin, among others. Furthermore, in some instances, growth factors such as glial cell line-derived neurotrophic factor (GDNF) and fibroblast growth factor (FGF) might be used to support spermatogonial stem cell cultures. However, the specific formulation can and will vary widely depending on the specific requirements of the testicular cells in question.

As various changes could be made in the above-described cells and methods without departing from the scope of the invention, it is intended that all matter contained in the above description and in the examples given below, shall be interpreted as illustrative and not in a limiting sense.

### EXAMPLES

All patents and publications mentioned in the specification are indicative of the levels of those skilled in the art to which the present disclosure pertains. All patents and publications are herein incorporated by reference to the same extent as if each individual publication was specifically and individually indicated to be incorporated by reference.

The publications discussed throughout are provided solely for their disclosure before the filing date of the present application. Nothing herein is to be construed as an admission that the invention is not entitled to antedate such disclosure by virtue of prior invention.

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Example 1. Generation of Primary Spermatocytes from dSPGs

In this study, the inventors demonstrate that differentiating spermatogonia can be induced to continue the spermatogenesis in vitro to produce sperm cells using a process developed by the inventors (see FIG. 1 and FIG. 2 for process of spermatogenesis). The process starts by sorting and purifying differentiating spermatogonia. The differentiating spermatogonia can be obtained from testicular tissue harvested from male subjects, testicular tissue grown in vitro as described in International Application No PCT/US2023/069593, the disclosure of which is incorporated herein by reference. The differentiating spermatogonia can also be obtained using an in vitro process of SSC culture and production of differentiating spermatogonia as described in International Application No PCT/US2023/069593, the disclosure of which is incorporated herein by reference.

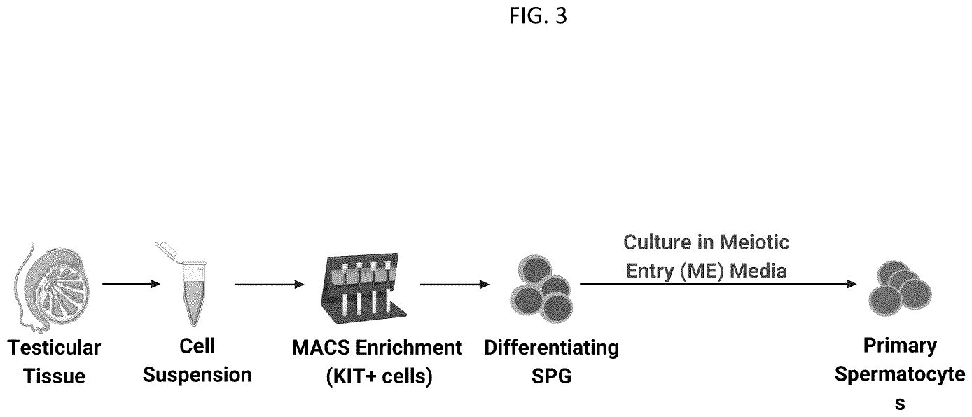

In this study, differentiating SPGs were isolated from testicular tissue harvested from human individuals (FIG. 3). In short, testicular tissue was placed on ice after harvesting and the outer layers of tissue, the epididymis, and the tunica albuginea were removed. The resulting testicular tissue was then minced with razor blades and incubated for 5 minutes at 37° C. in 1 mg/mL Collagenase type IV in PBS. Normally a higher concentration of enzyme is used. The resulting cells were then strained through a 40 μm cell strainer. The flow through was discarded and the tissue from the strainer was transferred into a fresh tube with 0.1% trypsin or TrypLE™ Express Enzyme (1×). The 0.1% trypsin and the use of TrypLE Express enzyme were critical for the performance of the cells and their ability to form round spermatids. The tissue was incubated for 30 minutes at 37° C. with gentle agitation. An equal volume of MEM-a media supplemented with 10% KSR and 1% Pen/strep was then added to the tissue and the contents of the tube were then strained through a 40 μm cell strainer. The flow through was then spun at 300 g for 5 minutes, and the cell pellet was resuspended in MACS buffer followed by MACS isolation of KIT+ (CD117) cells (differentiating spermatogonia) following the manufacturer's instructions.

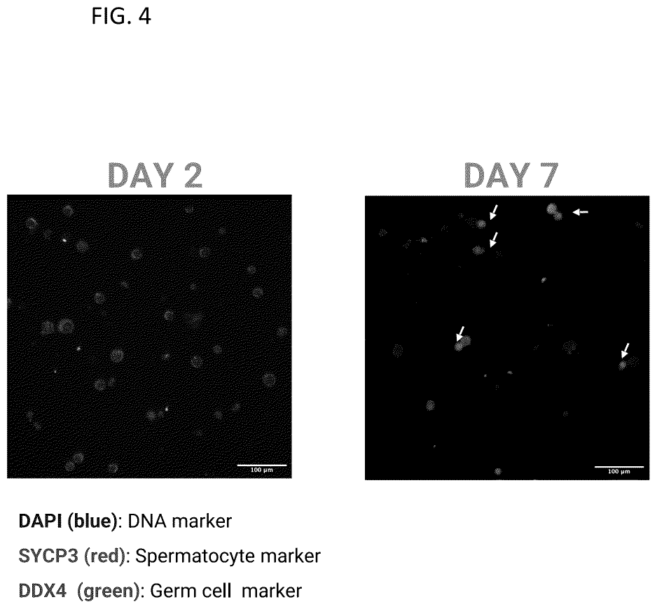

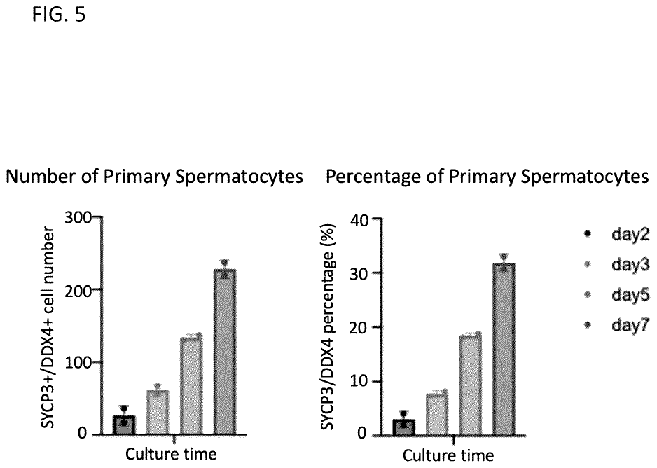

Cultures comprising the isolated differentiating spermatogonia were plated in 96 well plates in meiotic entry (ME) media with 0.25 μg/cm² recombinant laminin iMatrix 511 for 2, 3, 5, and 7 days designed to induce the cells to enter meiosis and form primary spermatocytes (4N). The wells were then stained and imaged with the EVOS m7000 imaging system and analyzed using CellProfiler and graphpad software (FIG. 4).

These results show that primary spermatocytes were generated from primary KIT+spermatogonia (FIG. 4 and FIG. 5). Therefore, the inventors show, for the first time, that human differentiating SPGs can be induced to enter meiosis. Importantly, primary spermatocytes were generated from primary KIT+spermatogonia even when these experiments were repeated with meiotic entry media without the three ligands: BMP2,4,7 (20 ng/mL each), Retinoic Acid (1 uM), Activin A (100 ng/ml).

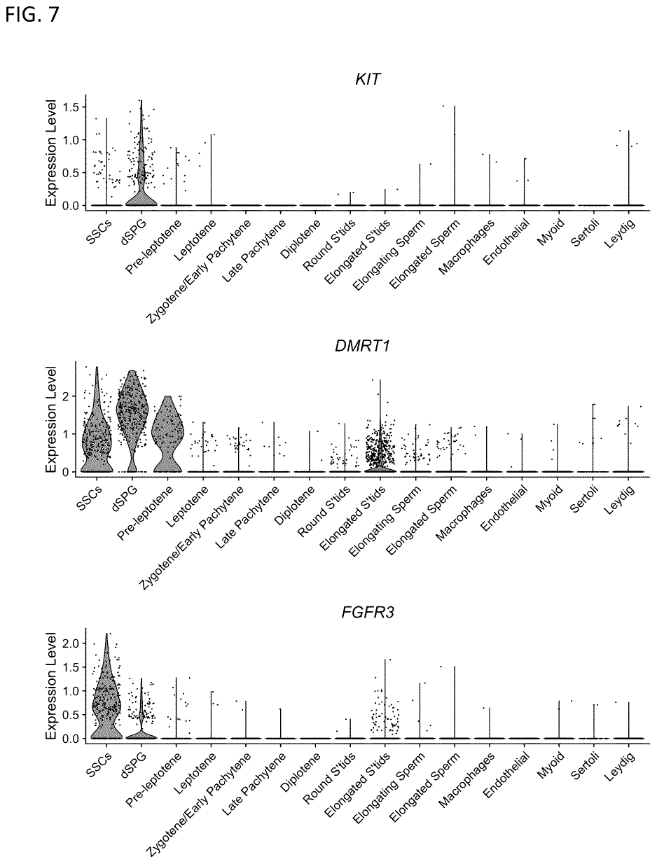

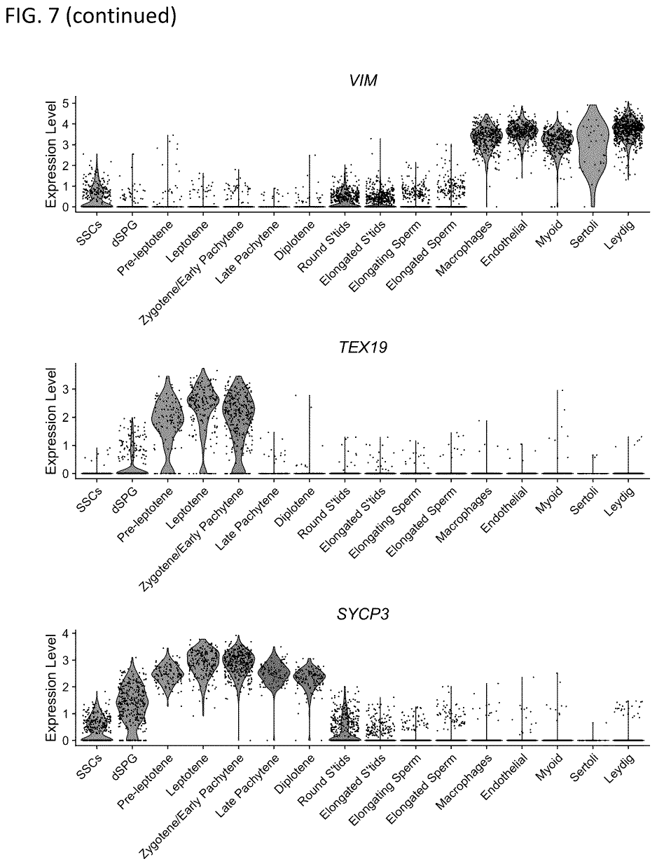

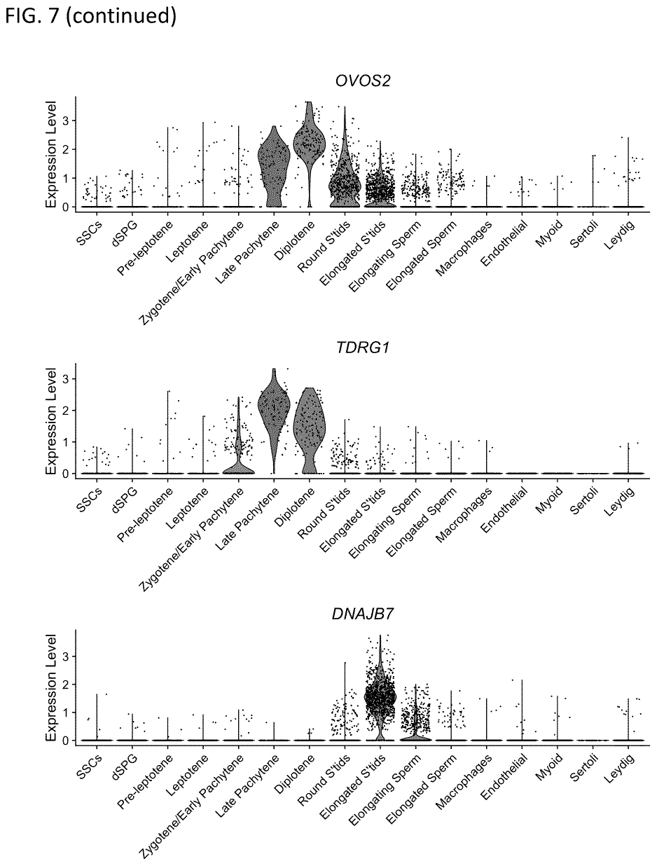

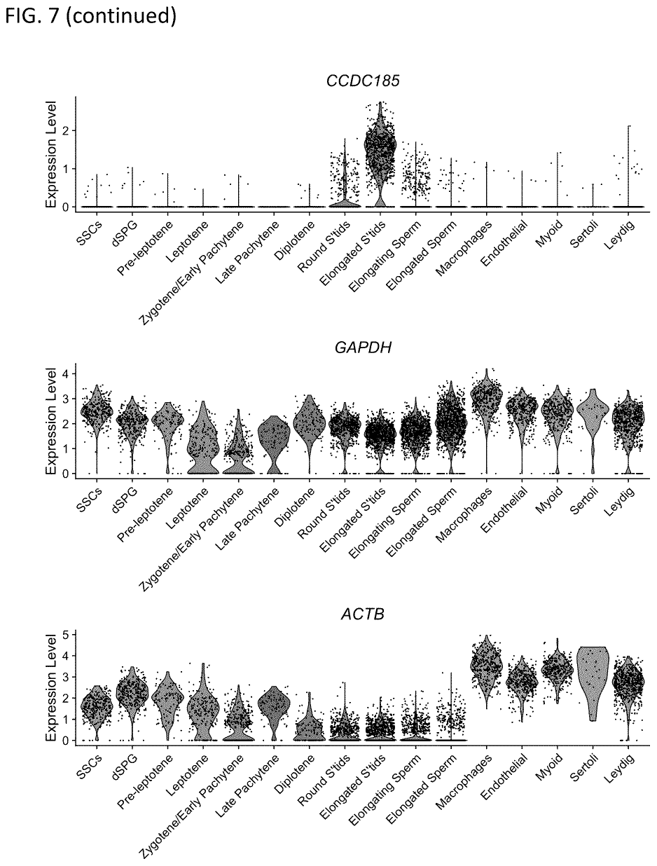

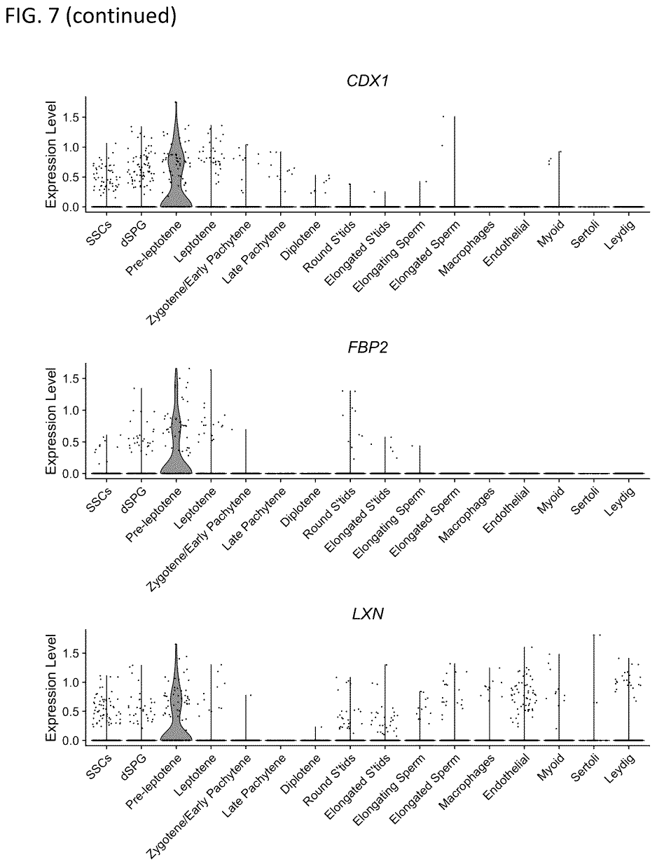

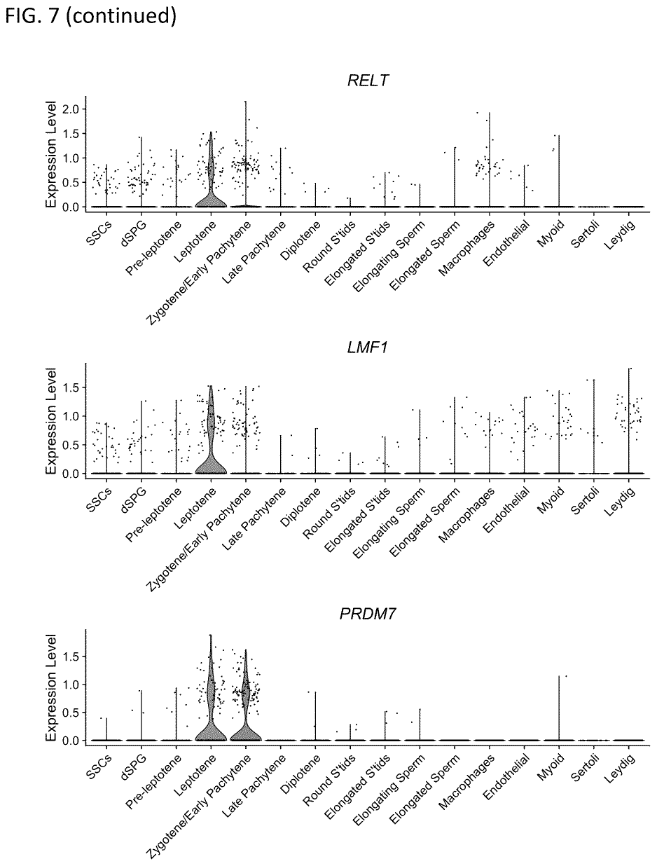

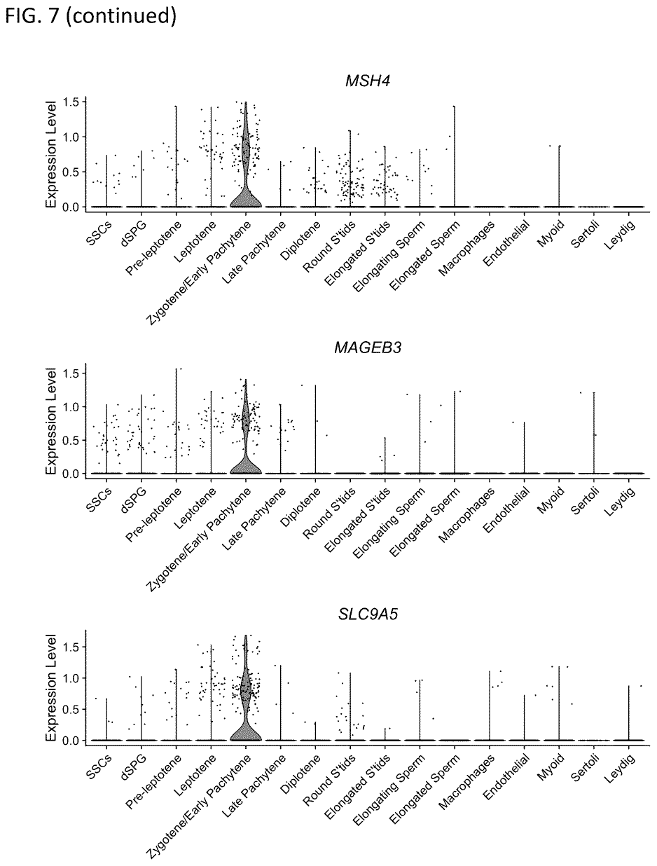

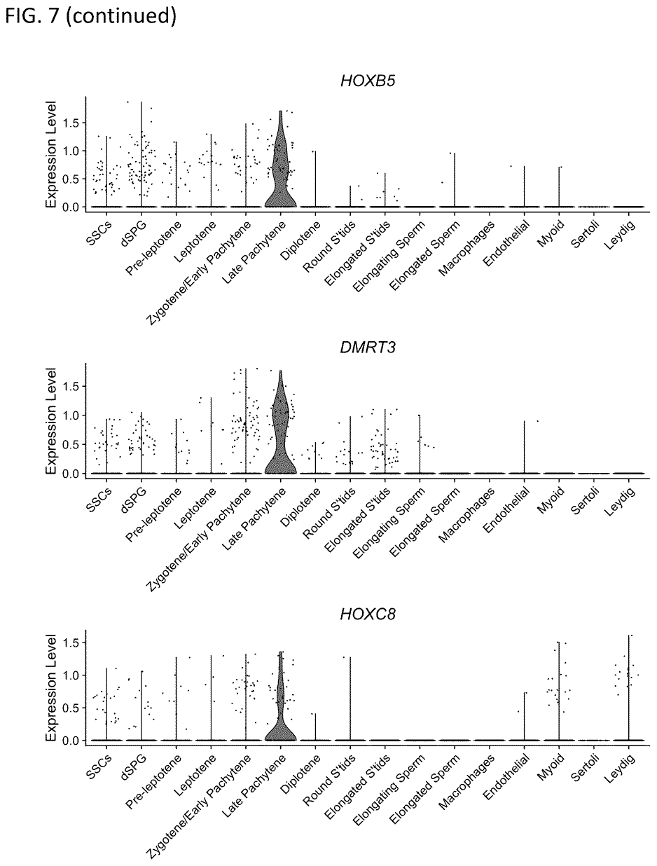

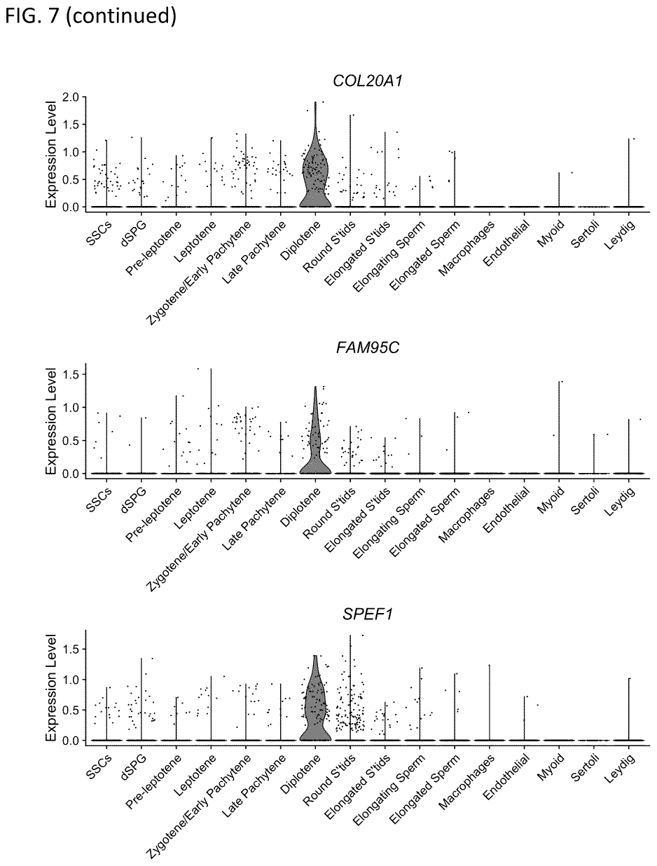

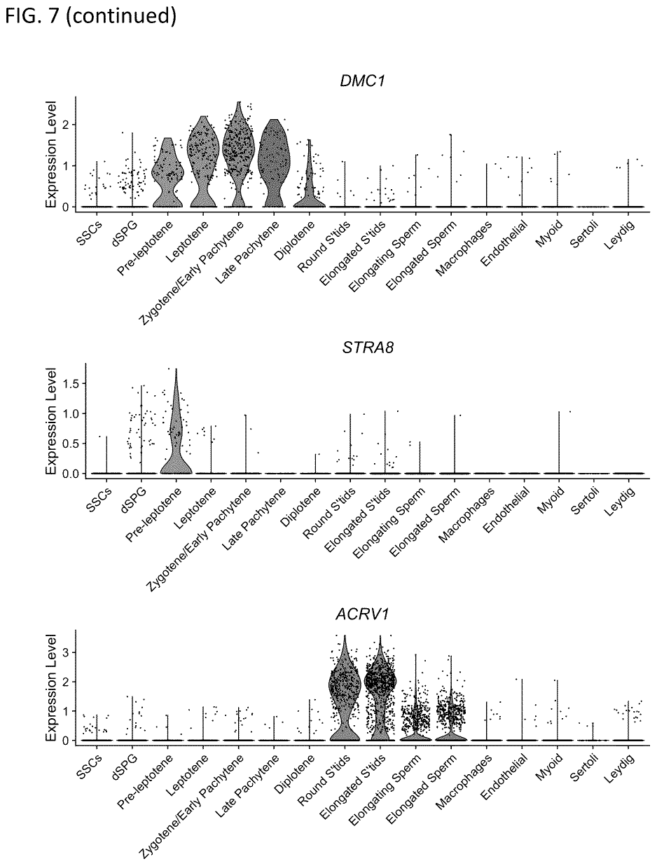

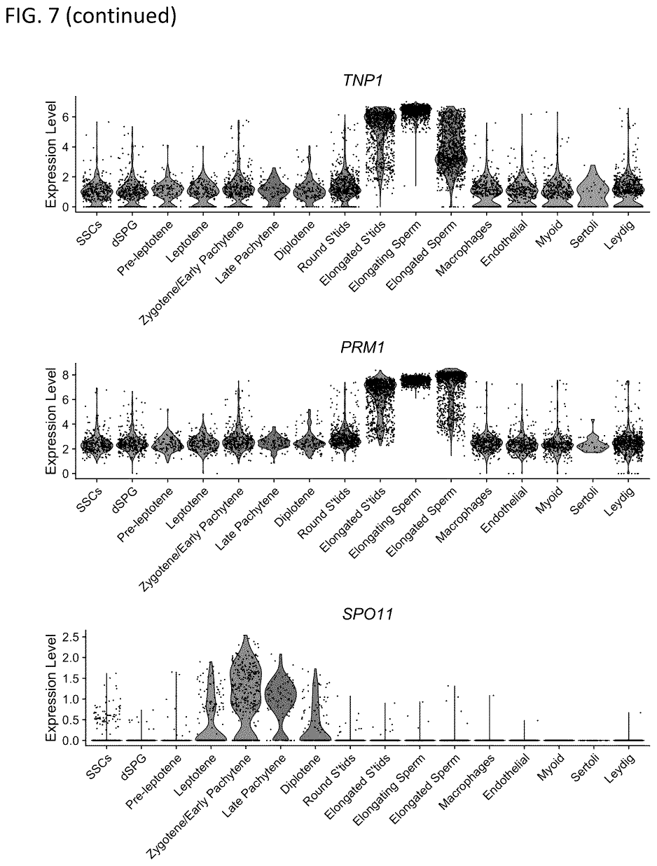

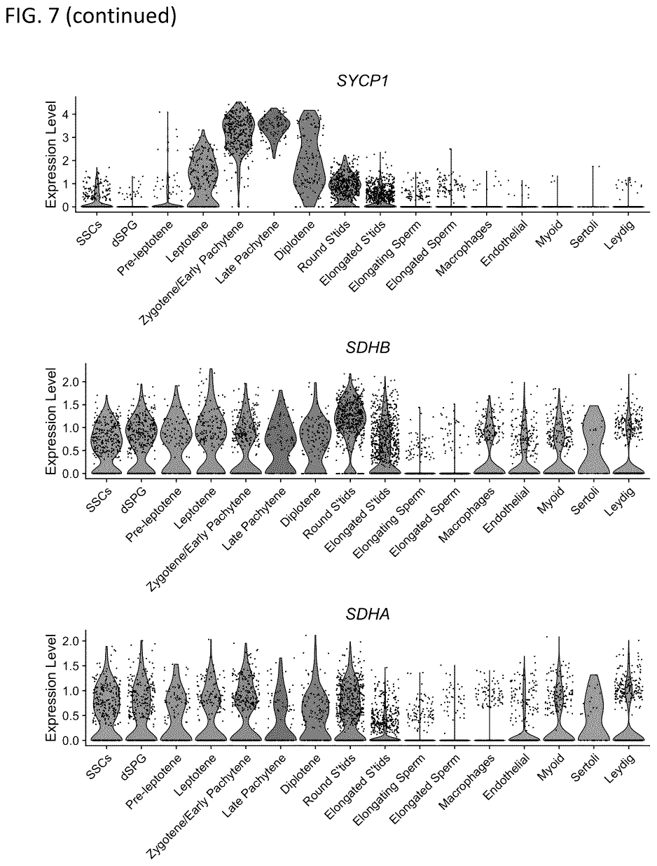

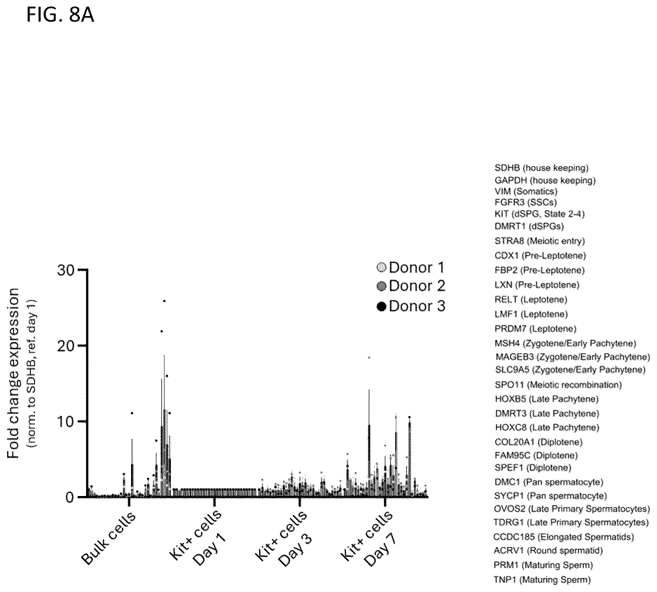

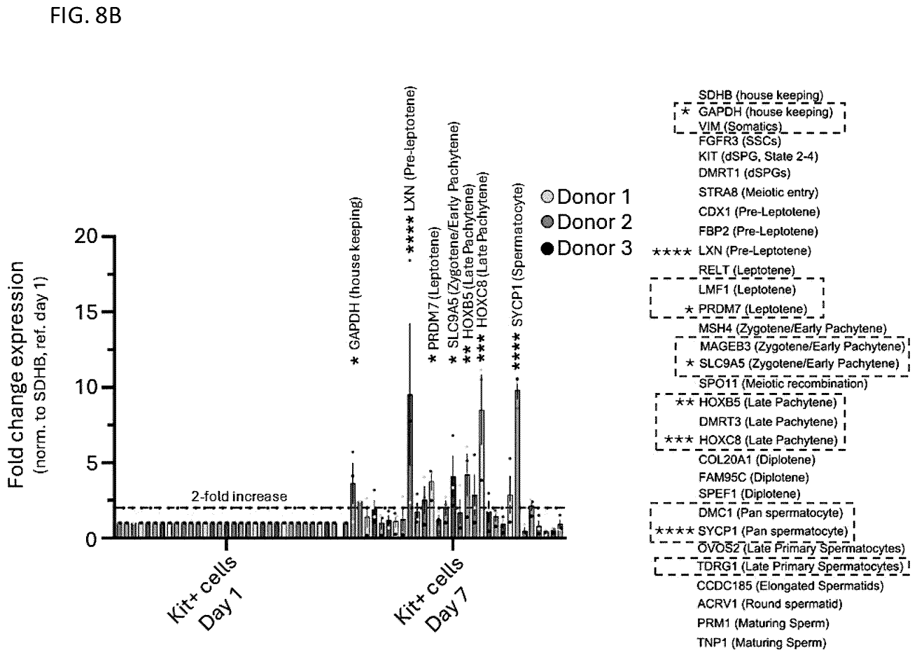

To confirm these results and further characterize meiotic entry and progression of KIT+ differentiating SPGs into meiosis in vitro, RT-qPCR gene expression analysis was performed to identify a set of target genes to allow for interrogation of transcriptional changes during in vitro spermatogenesis. By way of background, FIG. 6 shows the stages and duration of meiosis in differentiating SPGs. Cells isolated from KIT+ MACS isolation (Miltenyi, 130-091-332 as previously described) were counted and plated in ‘Meiotic Entry’ media (MEM-α, 10% KSR, 1% Pen/Strep, BMP2,4,7 (20 ng/ml each), Retinoic Acid (1 uM), Activin A (100 ng/ml) in suspension for 7 days. 500k cells were removed from the cultured population, pelleted with centrifugation (300 g, 5 minutes) and frozen in liquid nitrogen on day 1 (day of MACS isolation), day 3 and day 7. Cell pellets were held at −80° C. until RNA isolation. RNA was isolated using a commercially available RNA isolation kit (Zymo Research, R1055), residual genomic DNA was degraded (ThermoFisher, AM1907) and cDNA synthesis was performed (Invitrogen, 11-754-050), all according to manufacturer's instructions. Publicly available single cell RNA sequencing data set (Guo. et al, 2020) was used to identify differentially expressed genes across all stages of spermatogenesis, included meiotic entry and progression (FIG. 7) Using these data we designed and validated primers (Table 1) to allow the quantitative evaluation of cultured cells over the course of 7 days (FIG. 8 and FIG. 8).

TABLE 1
Primers used to determine transcriptional
changes during in vitro spermatogenesis.

| Gene | Cell Type/ | |
|---|---|---|
| Target | Stage | Primer Sequence |
| KIT | Differen- | F_GCACAATGGCACGGTTGAATGT |
| | tiating | (SEQ ID NO: 1) |
| | spermatogonia | R_AAAGGAGTGAACAGGGTGTGGG |
| | | (SEQ ID NO: 2) |
| DMRT1 | Differen- | F_TGGTCAGACAGGAAACCAGTGG |
| | tiating | (SEQ ID NO: 3) |
| | spermatogonia | R_GGGAGGCGGGTAGTAAGAATGC |
| | | (SEQ ID NO: 4) |
| FGFR3 | Spermatogonia | F_CAAGGTGTACAGTGACGCA |
| | Stem Cells | (SEQ ID NO: 5) |
| | | R_AAGGAGAGAACCTCTAGCTCC |
| | | (SEQ ID NO: 6) |
| VIM | Somatic Cells | F_ATGTTGACAATGCGTCTCTGGC |
| | | (SEQ ID NO: 7) |
| | | R_CTGTTCCTGAATCTGAGCCTGC |
| | | (SEQ ID NO: 8) |
| Tex19 | Early Primary | F_GTCAGCATGCGGTATGAGGAAG |
| | Spermatocytes | (SEQ ID NO: 9) |
| | | R_CCAGTTGTCTTCTTCCCAGTCC |
| | | (SEQ ID NO: 10) |
| SYCP3 | Early Primary | F_TGGGAAGCCGTCTGTGGAAG |
| | Spermatocytes | (SEQ ID NO: 11) |
| | | R_CCTACGTTTCTCAATGACTGC |
| | | AGTCT |
| | | (SEQ ID NO: 12) |
| OVOS2 | Late Primary | F_CGTCACCTCAAGTGCCACAAC |
| | Spermatocytes | (SEQ ID NO: 13) |
| | | R_TTCTTCAGGTGGGACTCCAGAG |
| | | (SEQ ID NO: 14) |
| TDRG1 | Late Primary | F_CCATTCTCCCATCCCAGGAAGG |
| | Spermatocytes | (SEQ ID NO: 15) |
| | | R_TGCTGGAGGTTTCAGAGATGGG |
| | | (SEQ ID NO: 16) |
| DNAJB7 | Elongated | F_GAAGTAGCTGAGGCATACGAGG |
| | Spermatids | TATTATC |
| | | (SEQ ID NO: 17) |
| | | R_TGGAATGTGAAGCCGTACTCACA |
| | | (SEQ ID NO: 18) |
| CCDC185 | Elongated | F_TCAGGGTAGAAAAGGCACAGGG |
| | Spermatids | (SEQ ID NO: 19) |
| | | R_CCTCTTGAACTTTTGCGAGGGC |
| | | (SEQ ID NO: 20) |
| GAPDH | House | F_AAGGGTCATCATCTCTGCCC |
| | keeping | (SEQ ID NO: 21) |
| | | R_CATGGACTGTGGTCATGAGT |
| | | (SEQ ID NO: 22) |
| ACTB-2 | House | F_GCCGCCAGCTCACCAT |
| | keeping | (SEQ ID NO: 23) |
| | | R_TCGTCGCCCACATAGGAATC |
| | | (SEQ ID NO: 24) |
| CDX1 | pre-leptotene | F_GGTAAGACTCGGACCAAGGACAAG |
| | primary | (SEQ ID NO: 25) |
| | spermatocytes | R_TCCGCCGGATTGTGATGTAACG |
| | | (SEQ ID NO: 26) |
| FBP2 | pre-leptotene | F_GTTAACGTGACGGGAGATGAGGT |
| | primary | (SEQ ID NO: 27) |
| | spermatocytes | R_CTTCTGAGACCAGGACGCAGG |
| | | (SEQ ID NO: 28) |
| LXN | pre-leptotene | F_TGAAGGAACCGCTAGAAGCACA |
| | primary | (SEQ ID NO: 29) |
| | spermatocytes | R_CACAGGCAACCCAGGCTAAATG |
| | | (SEQ ID NO: 30) |
| RELT | leptotene | F_TCAGGGCGAGATCACCATCTTG |
| | primary | (SEQ ID NO: 31) |
| | spermatocytes | R_GACACCATTGAACTTGTCCGCTG |
| | | (SEQ ID NO: 32) |
| LMF1 | leptotene | F_AGACCTCACCTGCATGGACTTC |
| | primary | (SEQ ID NO: 33) |
| | spermatocytes | R_AGCTCGATGAAGTGGTTGCTGA |
| | | (SEQ ID NO: 34) |
| PRDM7 | leptotene | F_GGAGACTGGGAGAAAACTCGCT |
| | primary | (SEQ ID NO: 35) |
| | spermatocytes | R_GGTGACACATGAAAGCTGGTCG |
| | | (SEQ ID NO: 36) |
| MSH4 | Zygotene/ | F_CAGGGTAGTGAACAGACAGCCA |
| | Early | (SEQ ID NO: 37) |
| | Pachytene | R_AAGTCGTCTACTCCCTCCAGGA |
| | spermatocytes | (SEQ ID NO: 38) |
| MAGEB3 | Zygotene/ | F_CTCACAGGACCCAAACATCCCT |
| | Early | (SEQ ID NO: 39) |
| | Pachytene | R_CAAGAACCTCACTTCGCTGCTG |
| | spermatocytes | (SEQ ID NO: 40) |
| SLC9A5 | Zygotene/ | F_TAACCGTGGAGTCTGAGGAGGA |
| | Early | (SEQ ID NO: 41) |
| | Pachytene | R_CCCTCTTGGAGAACCTCACTGG |
| | spermatocytes | (SEQ ID NO: 42) |
| HOXB5 | Late | F_GCTTCGGAGATGGAGTCCCTTT |
| | Pachytene | (SEQ ID NO: 43) |
| | spermatocytes | R_GACCAAGCCCCTAGGAGACTTG |
| | | (SEQ ID NO: 44) |
| DMRT3 | Late | F_CAGTGACAAAGACACTGACCAGAGG |
| | Pachytene | (SEQ ID NO: 45) |
| | spermatocytes | R_TCCTCCACGGACACTATCTCAGG |
| | | (SEQ ID NO: 46) |
| HOXC8 | Late | F_ATCCCGACTGTAAATCCTCCGC |
| | Pachytene | (SEQ ID NO: 47) |
| | spermatocytes | R_GAGGCTGGGAGACGAGTTTTGA |
| | | (SEQ ID NO: 48) |
| COL20A1 | Diplotene | F_GGAAGAGAGCAAGTTCAAGCAAGC |
| | Spermatocytes | (SEQ ID NO: 49) |
| | | R_ACTCTCTCCACTTCATCTGCAGC |
| | | (SEQ ID NO: 50) |
| FAM95C | Diplotene | F_CAGGTCCATAAAGCTCGGGTGA |
| | Spermatocytes | (SEQ ID NO: 51) |
| | | R_TGCCTTAGGTCCAGCTGTACAC |
| | | (SEQ ID NO: 52) |
| SPEF1 | Diplotene | F_TAGCGATGGAGTCCTTGTTGCA |
| | Spermatocytes | (SEQ ID NO: 53) |
| | | R_TGTTCAGATGACCCCAGTTGCT |
| | | (SEQ ID NO: 54) |
| DMC1 | Pan | F_GTCTCCAGGCTCCGTGTTCTAG |
| | Spermatocyte | (SEQ ID NO: 55) |
| | | R_CCCACAGTCTCCCCGAGATTTT |
| | | (SEQ ID NO: 56) |
| STRA8 | Meiotic | F_GGCATGCAAGCAGCTTAGAGG |
| | Entry | (SEQ ID NO: 57) |
| | | R_ACCAAGGGGAGGAACCATTCTG |
| | | (SEQ ID NO: 58) |
| ACRV1 | Round | F_CAAGGGTGTGAGAACATGTGCC |
| | Spermatid | (SEQ ID NO: 59) |
| | | R_TGAGTCAAAACAAGCAAGGGCC |
| | | (SEQ ID NO: 60) |
| TNP1 | Maturing | F_ATGGCATGAGGAGGAGCAAGAG |
| | Sperm | (SEQ ID NO: 61) |
| | | R_TCATCGCCCCGTTTCCTACTTT |
| | | (SEQ ID NO: 62) |
| PRM1 | Maturing | F_CACATCCACCAAACTCCTGCCT |
| | Sperm | (SEQ ID NO: 63) |
| | | R_TATTGACAGGCGGCATTGTTCCTT |
| | | (SEQ ID NO: 64) |
| SPO11 | Meiotic | F_GCTGTTGCTGTGCCATCGAATA |
| | Recombination | (SEQ ID NO: 65) |
| | | R_GTTGTCATCTAGGAGCCGCTGA |
| | | (SEQ ID NO: 66) |
| SYCP1 | Pan | F_GCCCTTTGCATTGTTCGTACCAC |
| | Spermatocyte | (SEQ ID NO: 67) |
| | | R_AAGAAAGTGGAATCGCCTCCCA |
| | | (SEQ ID NO: 68) |
| SDHB | House | F_GCATCTGTGGCTCTTGTGCAAT |
| | Keeping | (SEQ ID NO: 69) |
| | | R_ATACTGCTGCTTGCCTTCCTGA |
| | | (SEQ ID NO: 70) |
| SDHA | House | F_TGCCAGGGAAGACTACAAGGTG |
| | Keeping | (SEQ ID NO: 71) |
| | | R_AGTGCTCCTCAAAGGGCTTCTT |
| | | (SEQ ID NO: 72) |

Additionally, meiotic chromosome spreads were performed to determine the stage of meiosis at which the primary spermatocytes are in after this process. The protocol used for the chromosomal spread was as follows:

### Meiotic Spread:

1. Wells in a 96-well plate were coated with poly-L-lysine before or early the day of chromosome spread.
   Added 0.01% poly-L-lysine solution at 1 ml/25 cm\hat{ }2 and rocked gently to ensure even coating of the culture surface.
   After 5 min, removed solution and thoroughly rinsed the well with ultrapure ddH2O.
   Air dried for at least 2 h before using.
2. HEB buffer was prepared with fresh DTT and PMSF, kept on ice.
3. Sucrose solution (100 mM) was prepared and kept on ice.
4. Fixative solution was prepared and kept at room temperature protected from light.
5. Fixative solution was added to the wells for chromosome spread for pre-treatment, enough to cover the whole bottom, left at room temperature in the chemical hood.
6. Cells were collected and counted for experiment, spun down at 300×g for 5 min.
7. Cells were resuspended in 1e6/1 ml of HEB buffer and keep on ice for 30 min.
8. After HEB treatment, nuclei were spun down at 200×g for 5 min, with slow deceleration.
9. Nuclei were resuspended in 1e7/ml of 100 mM sucrose and kept on ice.
10. Fixative solution was removed from wells and just enough was left to cover the bottom of the well, then, 8 μl of nuclei/sucrose solution was added to the fixative drop and the reaction was mixed by pipetting a few times.
11. The plate with nuclei was kept in a humidity chamber for 4 h, the spread was allowed to dry slowly but not completely.
12. Wash solution was prepared, and each well was rinsed twice, each time for 2 min.
13. The wells were then air dried for 15 min at room temperature before proceeding with IF staining as described herein below, imaging or the plates were stored at-80C for future use.

### IF Staining and Imaging:

14. EdU Click-iT™ reaction cocktails were prepared as instructed by the manual (C10337). Spreads were rinsed with PBS, incubated with 80 ul/well reaction cocktails for 30 min in dark.
15. After incubation, EdU reaction cocktails were removed, and the wells were washed with PBS for 3 times.
16. Wells were blocked in PBS super block solution (Catalog No. PI37515) with 5% donkey serum for 45 min at room temperature.
17. Overnight incubation with primary antibodies: mouse anti-γH2AX (1:800; Millipore sigma 05636) and goat anti-SYCP3 (1:400; Millipore sigma HPA039635).
18. The spreads were washed 3 times with PBS and subsequently incubated for 1 h with secondary antibodies: Alexa Fluor 594 Rabbit and Alexa Fluor plus 647 Mouse (A-21207 and A-32787TR, Invitrogen) at 1:500 dilution as well as Hoechst (Invitrogen 33342) at 1:1000 dilution.
19. The spreads were washed 3 times with PBS and kept in PBS for subsequent imaging with ImageXpress Micro-confocal (Molecular Devices).

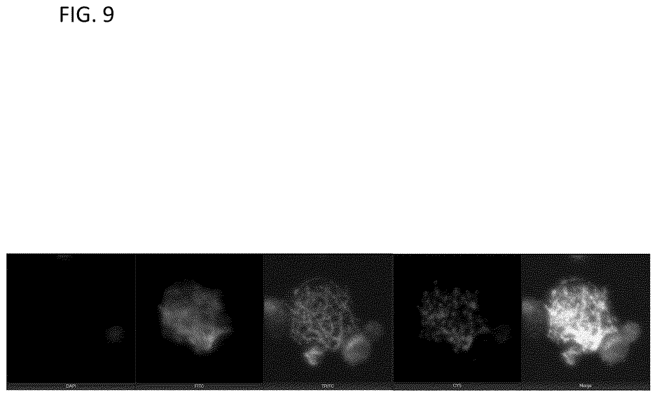

As seen in FIG. 9, the meiotic spreads show that the in vitro cultured Kit+ cells resemble zygotene spermatocytes. Un-synapsed homologous chromosomes were marked by strip pattern of SYCP3 and clear dotted pattern of H2AX.

Based on the collective results, the inventors did not identify media conditions that can induce production of secondary spermatocytes from the pre-leptotene primary spermatocytes. However, during the extensive experimentation, the inventors surprisingly observed that round spermatids were nevertheless being produced in the ME media. Further, the round spermatids and the zygotene cells were obtained in base media even when no secondary spermatocytes were observed. This can be seen by removing all round spermatids and observing that new round spermatids appeared after a few days, even without defined ligands.

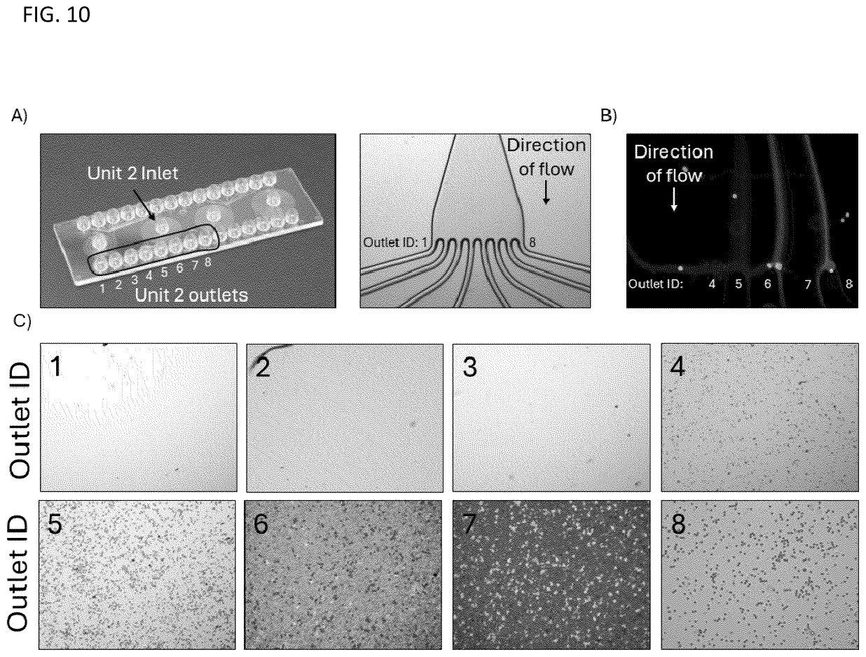

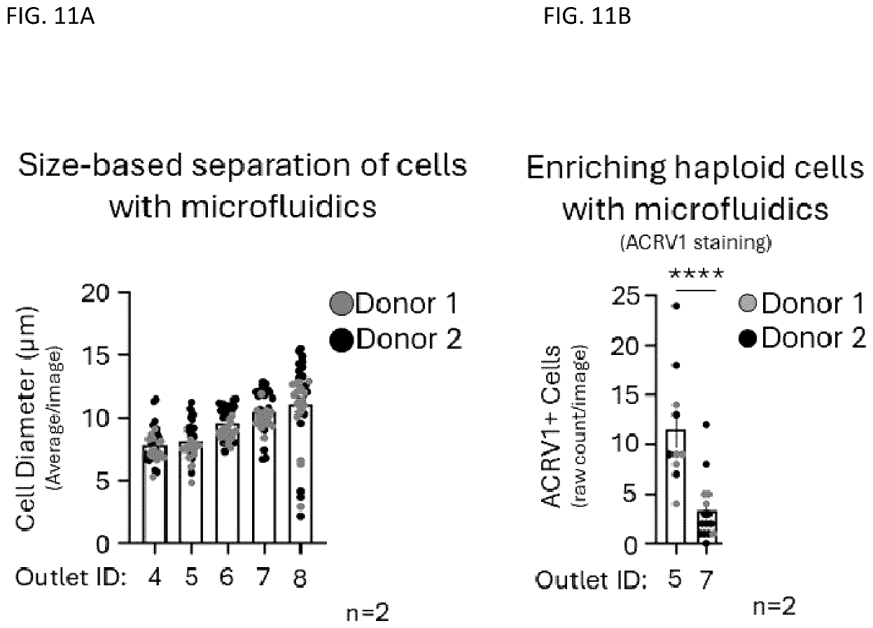

Round spermatids were removed using a spiral microfluidic chip. In short, 6 μm (blue), 10 μm (green) and 15 μm (red) fluorescent microspheres were formulated in 1×PBS at 0.333×106 particles/mL each (1×106/mL final concentration) and loaded into a microfluidic syringe pump (New Era Pump System Inc, Part #1000-US) (FIG. 10). The particle-containing syringe was connected to the inlet of the 8-outlet spiral channel particle sorter (Microfluidic ChipShop, Item #382, sorting unit 2. FIG. 10, panels A and B) and ran at a rate of 1.5 mL/minute, allowing the size-based separation of the fluorescent particles. Each outlet (1-8) was collected, and the harvested fluorescent particles were imaged using fluorescent microscopy (ThermoFisher EVOS M7000, cat #AMF7000) (FIG. 10, Panel C). Cell suspension from human testicular tissue was suspended in 1×PBS at a concentration of 1×106/mL and loaded into a 10 ml syringe and ran through an 8-outlet spiral channel sorter (Microfluidic ChipShop, Item #382) at 1.5 mL/minute. To collect cells and avoid clogging, outlets 2-8 were connected to an 8-channel syringe pump (New Era Pump System Inc, Part #NE-1800-US) which withdrew at a rate of 0.1875 mL/minute, pulling equally on all outlets. Collected outlets were plated in 96-well imaging dishes and immediately imaged with brightfield microscopy to measure cell diameter (FIG. 11). Cells from outlets 5 and 7 were then fixed and stained with a haploid round spermatid marker, ACRV1 (Lifetech, Cat: 14040-1-AP at 1:400, FIG. 11). Haploid enrichment was quantified by counting total number of ACRV1+ cells in outlets 5 and 7.

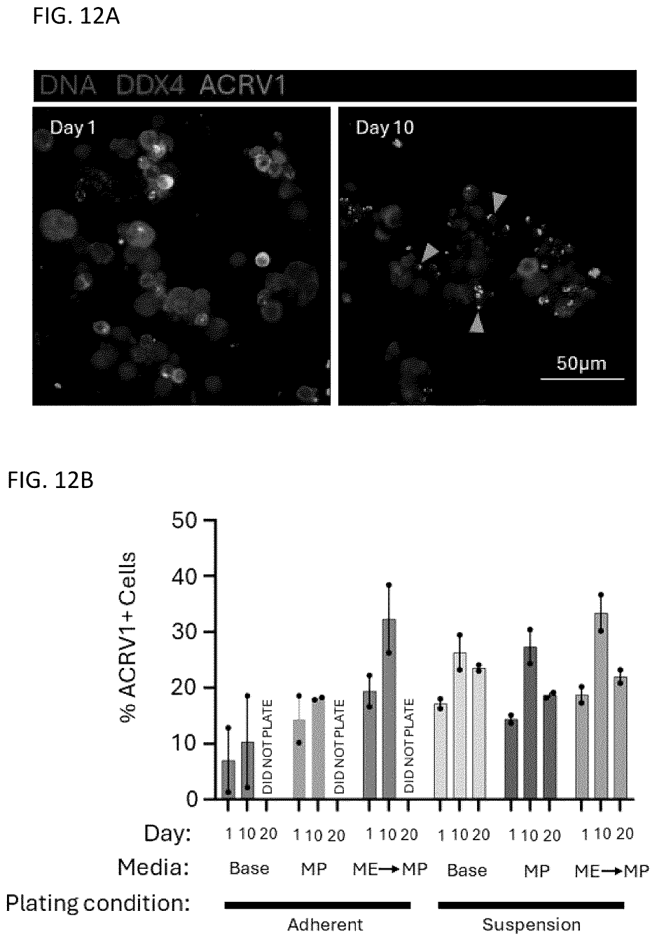

Bulk (un-enriched) cells were isolated from fresh human testicular tissue and plated in the base media (MEM-α, 10% KSR, 1% Pen/Strep, 1× Pluronic acid—see Table 2 for vendor and catalog numbers), ‘Meiotic Entry’ (ME) media (Base media, BMP2,4,7, Retonic Acid, Activin A—see Table 2 for vendor, catalog numbers and concentrations) for 10 days or ME media for 3 days and then ‘Meiotic progression’ (MP) media (FSH, BPE, Testosterone—see Table 2 for vendor, catalog numbers and concentrations) for 7 days. All conditions were plated as suspension or adherent conditions (adding solubilized laminin to the culture media—see Table 2 for vendor, catalog and concentration). Standard methods were used to fix and stain with DDX4 (Abcam, 27591), a germ cell marker, and ACRV1 (Lifetech, 14040-1-AP), a round spermatid marker, on days 1 (24 hr after plating) day 10, and day 20. Fluorescent microscopy was used to visualize (FIG. 12) and quantify (FIG. 12) ACRV1+ cells at each time point. The increased ACRV1+ cells in these data demonstrate the in vitro production of round spermatids over the course of 10 days.

TABLE 2
Materials and reagents

| Material | Vendor | Catalog | Concentration/Dilution | |
|---|---|---|---|---|
| MEM-α | Gibco | 12567-056 | NA | |
| KSR | Gibco | 10828-028 | 10% | |
| Pen/Strep | Gen Clone | 25-512 | 1% | |
| BMP2 | R&D Systems | 355-BEC-010/CF | 20 | ng/mL |
| BMP4 | R&D Systems | 314-BPE-010/ | 20 | ng/mL |
| | | 314-BPE-050 | | |
| BMP7 | R&D Systems | 354-BP-010/CF | 20 | ng/mL |
| Retinoic Acid | Sigma Aldrich | R2625-100MG | 1 | μM |
| Activin A | R&D Systems | 338-GMP-010 | 100 | ng/mL |
| Follicle stimulating | Sigma Aldrich | F4021 | 200 | ng/mL |
| hormone (FSH) | | | | |
| Bovine Pituitary | Corning | 354123 | 50 | μg/mL |
| Extract | | | | |
| Testosterone | Sigma | PHR227 | 10 | μM |

| | | | |
|---|---|---|---|
| Mouse anti-DDX4 | Abcam | Ab27591 | 1:400 |
| antibody | | | |
| Rabbit anti-ACRV1 | Lifetech | 14040-1-AP | 1:200 |
| antibody | | | |
| Donkey anti- | ThermoFisher | A21202 | 1:800 |
| mouse488 | | | |
| Donkey anti-rabbit | ThermoFisher | A21207 | 1:800 |
| 594 | | | |
| Hoechst 33342 | Invitrogen | H3570 | 1:10,000 |
| iMatrix-511 | Advanced biomatrix | 5344 | 1:2,500 |
| (Laminin) | | | |

Accordingly, the differentiating SPGs used by the inventors, the pre-leptotene primary spermatocytes, both, or other yet to be identified precursor cells, when introduced into the ME culture media, were capable of spontaneously generating round spermatids through a yet to be discovered cell differentiation pathway. Further, it was observed that the round spermatids were produced in ME media even without the use of ligands. This suggests that the cells can spontaneously enter meiosis.

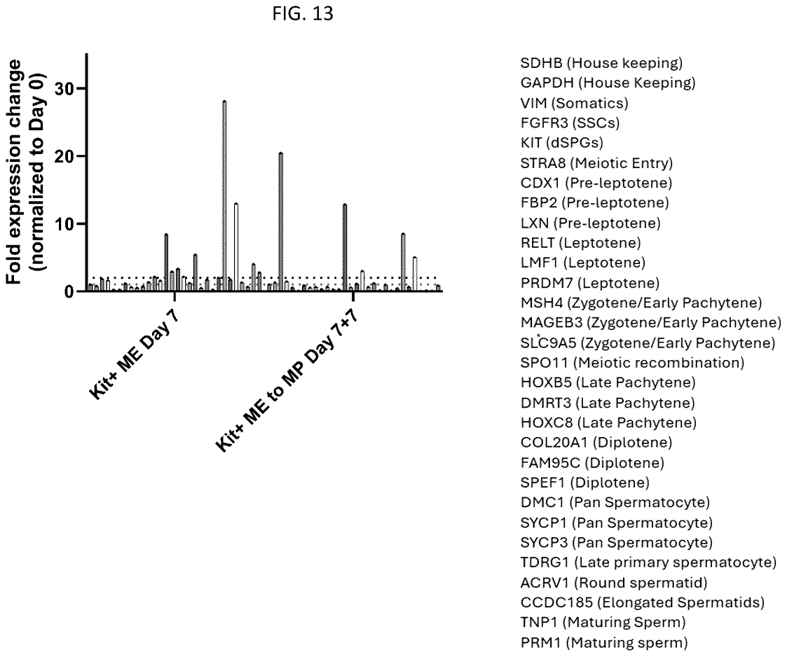

Importantly, cells grown in ME media then switched to MP media showed little to no generation of round spermatids. Cells were digested and sorted against KIT using MACS isolation techniques as previously described. Cells were harvested prior to culturing as a ‘Day 0’ control. Cells were then cultured in Meiotic Entry (ME) media for 7 days, and then resuspended in Meiotic Progression (MP) media for an additional 7 days (day 14). Cells were collected on days 7 and 14 for RNA isolation and RT-qPCR analyses. These data demonstrate little to no expression genes associated with advanced stages of spermiogenesis in the ME to MP condition using ACRV1 (round spermatid), CCDC185 (elongated spermatid), TNP1 (maturing sperm) or PRM1 (Maturing sperm) as markers (FIG. 13).

### Example 2. Culture Conditions for Development of Human SSCs or dSPGs to Flagellar Spermatids In Vitro

In this study, the inventors systematically evaluated a range of media conditions to enable, for the first time, the in vitro development of SSCs or human differentiating spermatogonia (dSPGs) through meiotic entry, meiotic progression to spermatocytes, meiotic completion to haploid round spermatids, and further development into elongated spermatids (see FIG. 1 and FIG. 2 for an overview of spermatogenesis). Prior to this invention, successful recapitulation of these key stages of human spermatogenesis in vitro had not been achieved, as the literature describes only limited or partial differentiation of human germ cells outside the body, with complete meiotic progression and spermatid formation previously demonstrated only in rodent models (see, e.g., Sato et al., Nature 2011; Zhou et al., Cell Stem Cell 2016).

SSCs can be obtained as described in PCT Application No PCT/US2023/069593, the disclosure of which is incorporated herein in its entirety. Differentiating spermatogonia were isolated from human testicular tissue obtained from cadaveric donors, as described in Example 1. Briefly, the tissue was processed to remove the tunica and expose the seminiferous tubules, which were then gently dissociated using a combination of collagenase IV and TrypLE enzymatic digestion. The resulting single-cell suspension was enriched for dSPGs using CD117 (KIT) magnetic-activated cell sorting (MACS).

The isolated dSPGs were subjected to a series of culture conditions designed to identify those that would support the full developmental sequence from dSPGs to round and elongated spermatids. The inventors tested a wide array of media foundations, including Minimum Essential Medium Alpha (αMEM), Advanced Protein-free Eukaryotic cell Line medium 2 (APEL2), Medium 199 (M199), and Dulbecco's Modified Eagle Medium/Nutrient Mixture F-12 (DMEM/F12), each supplemented with various factors such as specific growth factors, hormones, metabolic modulators, and pathway inhibitors. These included bone morphogenetic protein (BMP) family members, retinoic acid, lactate, conditioned media, 2-mercaptoethanol, activin A, follicle-stimulating hormone (FSH), testosterone, bovine pituitary extract (BPE), and WNT pathway modulators. The compositions of the principal media tested are shown in Tables 18-22.

Media #1 (Table 3) served as the basic medium, consisting of αMEM (or APEL2 or DMEM/F12) supplemented with 10% Knockout Serum Replacement (KSR) and 1% penicillin-streptomycin, and did not include additional hormones or specialized supplements. This provided a baseline for comparison with more complex or supplemented conditions.

TABLE 3

| Cell culture | | | |
|---|---|---|---|
| media #1 | Concentration | Vendor | Catalog |
| Alpha-MEM, APEL2, | 89% | Gibco | 12571048 |
| or DMEM/F12 | | | |
| Knockout Serum | 10% | Gibco | 10828028 |
| Replacement (KSR) | | | |
| Pen/Strep | 1% | Gibco | 15070063 |

Media #2 (Table 4) is a nutrient-rich medium based on the same αMEM/KSR/penicillin-streptomycin foundation as the base medium, but further supplemented with follicle-stimulating hormone (FSH, 20 ng/ml), a high concentration of testosterone (10 uM), and bovine pituitary extract (BPE, 50 ug/mL), and may use αMEM, APEL2, DMEM/F12, or M199 as the basal medium. The addition of these hormones and pituitary factors was intended to provide a supportive, nutrient-rich environment to serve as the foundation for identifying and optimizing the conditions required for meiotic progression and completion.

TABLE 4

| Cell culture media #2 | Concentration | | Vendor | Catalog |
|---|---|---|---|---|
| αMEM, APEL2, M199, | 89% | | Gibco | 12571048 |
| or DMEM/F12 | | | | |
| Knockout Serum | 10% | | Gibco | 10828028 |
| Replacement (KSR) | | | | |
| Pen/Strep | 1% | | Gibco | 15070063 |
| Follicle Stimulating | 20 | ng/mL | Sigma | F4021-10UG |
| Hormone (FSH) | | | Aldrich | |
| Testosterone 10 uM | 10 | uM | | |
| Bovine Pituitary Extract (BPE) | 50 | ug/mL | Corning | 354123 |

Media #3 (Table 5) is a nutrient-restricted medium, in which most of the nutrient-rich base was replaced with Earle's Balanced Salt Solution (EBSS, 90%), with only 10% αMEM and lower concentrations of KSR (1%). Like media #2, it was supplemented with FSH, testosterone, and BPE, but the overall nutrient content was substantially reduced, allowing assessment of how nutrient restriction influences meiotic entry and progression.

TABLE 5

| Cell culture media #3 | Concentration | | Vendor | Catalog |
|---|---|---|---|---|
| EBSS | 90% | | | |
| Alpha-MEM | 10% | | Gibco | 12571048 |
| Knockout Serum | 1% | | Gibco | 10828028 |
| Replacment (KSR) | | | | |
| Pen/Strep | 1% | | Gibco | 15070063 |
| Follicle Stimulating | 20 | ng/mL | Sigma | F4021-10UG |
| Hormone (FSH) | | | Aldrich | |
| Testosterone 10 uM | 10 | uM | | |
| Bovine Pituitary Extract | 50 | ug/mL | Corning | 354123 |

Media #4 (Table 6) is a highly nutrient-rich and complex formulation, containing αMEM/KSR as a base and a wide array of additional supplements, including 2-mercaptoethanol, glucose, lactic acid, pyruvic acid, bovine serum albumin, dihydrotestosterone (DHT), L-glutamine, fetal bovine serum, BPE, FSH, d-biotin, β-estradiol, melatonin, ascorbic acid, MEM vitamin solution, N-2 supplement, non-essential amino acids, and TGFB. This medium was used to test the effects of various rich media conditions on spermatogenesis, allowing for the evaluation of how extensive supplementation influences the development and differentiation of human germ cells in vitro.

TABLE 6

| Cell culture | | | | |
|---|---|---|---|---|
| media #4 | Concentration | | Vendor | Catalog |
| 2-Mercaptoethanol | 50 | uM | MilliporeSigma | M3148-25ML |
| D-(+)-Glucose | 6 | mg/mL | Thermo Fisher | A16828.36 |
| DL-Lactic Acid | 1 | ul/mL | Thermo Fisher | 412965000 |
| Pyruvic Acid | 200 | ug/mL | Thermo Fisher | A13875.22 |
| BSA | 5 | mg/mL | Sigma Aldrich | 10735086001 |
| DHT | 10 | uM | Sigma | D-073-1ML |
| L-Glutamine | 2 | mM | Thermo Fisher | 25030081 |
| FBS | 10 | ul/mL | Sigma Aldrich | F2379-250MG |
| KSR | 100 | ul/mL | Thermo Fisher | 12618013 |
| BPE | 50 | ng/ml | Corning | 354123 |
| FSH | 200 | ng/mL | Sigma Aldrich | F4021-10UG |
| d-Biotin | 10 | ug/mL | Sigma Aldrich | B4639-100MG |
| B-Estradiol | 30 | ng/mL | Selleckchem | S1709 |
| Melatonin | 1 | uM | Sigma Aldrich | M5250-250MG |
| Ascorbic Acid | 1 | mM | Matrix | 92375 |
| | | | Scientific | |
| MEM Vitamin | 1× | | Sigma Aldrich | M6895-100ML |
| N-2 | 1× | | R&D Systems | AR-009 |
| NEAA | 1× | | Sigma Aldrich | M7145-100ML |
| TGFb | 10 | ng/mL | STEMCELL | 78067 |

Technology

Media #5 (Table 7) is a nutrient-restricted medium formulated with 89% EBSS and 10% αMEM, supplemented with 1% KSR and 1% penicillin-streptomycin, and further enriched with BMP2 (20 ng/mL), BMP4 (20 ng/ml), BMP7 (20 ng/ml), retinoic acid (5 uM), and activin A (100 ng/ml). This medium is notable for its combination of morphogens and differentiation factors, and for its use of a nutrient-restricted base similar to media #3, but without FSH, testosterone, or BPE.

TABLE 7

| Cell culture | | | | |
|---|---|---|---|---|
| media #5 | Concentration | | Vendor | Catalog |
| EBSS | 89% | | Gibco | 14155063 |
| Alpha-MEM, | 10% | | Gibco | 12571048 |
| APEL2, or | | | | |
| DMEM/F12 | | | | |
| KSR | 1% | | Gibco | 10828028 |
| Pen/Strep | 1% | | Gibco | 15070063 |
| BMP2 | 20 | ng/mL | R&D Systems | 355-BEC-010/CF |
| BMP4 | 20 | ng/mL | R&D Systems | 314-BPE-050 |
| BMP7 | 20 | ng/mL | R&D Systems | 354-BP-10/CF |
| Retinoic Acid | 5 | uM | Sigma Aldrich | R2625-100MG |
| Activin A | 100 | ng/mL | R&D Systems | 338-GMP-010 |

In summary, media #1, #2, #3, #4, and #5 each utilize a base of αMEM, Knockout Serum Replacement (KSR), and penicillin-streptomycin, but differ substantially in their nutrient richness and the specific reagents added. Media #1 serves as a basic medium, containing only the core components without additional hormones or supplements, and provides a baseline for comparison. Media #2 is nutrient rich, containing a high proportion of αMEM and further supplemented with follicle-stimulating hormone (FSH), testosterone, and bovine pituitary extract (BPE), and serves as the foundation for identifying optimal conditions for meiotic progression and completion. Media #3 is nutrient restricted, with the majority of the medium composed of Earle's Balanced Salt Solution (EBSS) and only a small fraction of αMEM, yet it also includes FSH, testosterone, and BPE, creating a low-nutrient environment with targeted hormonal support. Media #4 is also nutrient rich but is distinguished by its extensive supplementation with a wide array of metabolic, hormonal, and antioxidant factors, allowing for the evaluation of how complex, highly supplemented conditions affect spermatogenesis. Media #5 is nutrient restricted, formulated primarily with EBSS and a small amount of αMEM, and is notable for the addition of multiple bone morphogenetic proteins (BMP2, BMP4, BMP7), retinoic acid, and activin A, without the inclusion of FSH, testosterone, or BPE. These differences in nutrient content and supplemental factors allowed for a systematic evaluation of how nutrient availability and specific signaling molecules influence human spermatogonial differentiation and meiotic development, with media #2 and #5 ultimately being used in later stages of the experimental workflow.

In addition to the different media formulations, a range of other experimental variables was systematically tested to optimize conditions for spermatogenesis (Tables 8-10). A wide range of reagents were tested in combination with the various media formulations, including insulin, IGF-1, WNT pathway modulators (e.g., IWR-endo-1), BAX inhibitor peptide, and others, at various concentrations. Plate modifications were also explored, including the use of various substrates and feeder cell layers, such as pre-plated mouse embryonic fibroblasts (MEFs), Matrigel, and Geltrex, to assess the impact of cell adhesion and microenvironmental support on germ cell development. The duration of culture in each medium/reagent was varied, with time points ranging from one to several days, to determine the optimal exposure periods for each stage of differentiation. Sequential use of multiple media during the culture process was also investigated, with protocols involving initial culture in one medium followed by transfer to a different medium to possibly mimic dynamic changes in the in vivo environment and to promote specific developmental transitions. Additional variables included the use of suspension versus adherent culture formats, and the presence or absence of specific growth factors or inhibitors at different stages. Collectively, these variables were systematically manipulated to identify the most effective combination of conditions for supporting human spermatogonial meiotic entry, progression, and completion to spermatids in vitro.

Based on all these experiments, it was discovered that distinct and sequentially tailored culture conditions were required to guide human germ cells through the major transitions of spermatogenesis in vitro. The experimental data and results for each stage, including meiotic entry, meiotic progression, and spermatid formation, are described in detail in Examples 3-5 herein below.

### Example 3. Entry of Human SSCs and dSPGs into Meiosis In Vitro

The meiotic entry experiments were designed to systematically evaluate a wide range of compounds and culture conditions for their ability to induce human differentiating spermatogonia (dSPGs) to enter meiosis. Entry into meiosis is recognized in the art as a major developmental milestone in spermatogenesis, representing the transition from mitotic proliferation to the initiation of meiotic prophase I and the commitment to gamete formation. In these experiments, dSPGs were typically cultured for one to seven days in the test conditions, with some protocols involving sequential use of different media or additives over this period. The primary readout was the fraction of cells that became EdU-positive and expressed early meiotic markers, indicating successful entry into meiosis.

Medium #5 (Table 7), a nutrient-restricted medium, was of particular interest as it supported a modest 20% of dSPGs entering meiosis when compared to other media formulations. A wide range of media and compounds were tested in combination with medium #5, including insulin, IGF-1, metabolic modulators (such as 2-deoxyglucose and oxythiamine), WNT pathway modulators (including WNT1, WNT3A, WNT-11, laduviglusib, IWR-endo-1, DKK1, DKK2), growth factors (EGF, MDK, CNTF, NRG1, HGF), and apoptosis inhibitors (BAX inhibitor peptide, BCL2). A sampling of these conditions is shown in Table 8.

Most of these individual compounds or combinations, when added to medium #5, resulted in only a modest fraction of the cells entering early meiosis, with typical rates ranging from 15% to 29% EdU-positive early meiotic cells. For example, the use of IGF-1, 2DG, oxythiamine, and various WNT ligands or inhibitors produced early meiotic entry rates in this modest range, and none of these conditions alone were sufficient to drive robust meiotic entry.

Significant improvements were observed in the final set of conditions, corresponding to the last five rows of Table 8. In these protocols, cells were cultured in medium #5 supplemented with both insulin (50 ug/mL) and IWR-endo-1 (3 uM), with careful control of the timing and sequence of media changes. The duration of culture in these optimized protocols was typically four days, with some variations involving one to three days in the supplemented medium #5 followed by transfer to another nutrient-rich medium (media #2) for the remaining days to test the effect of duration of culture in the amended media #5 (media #6).

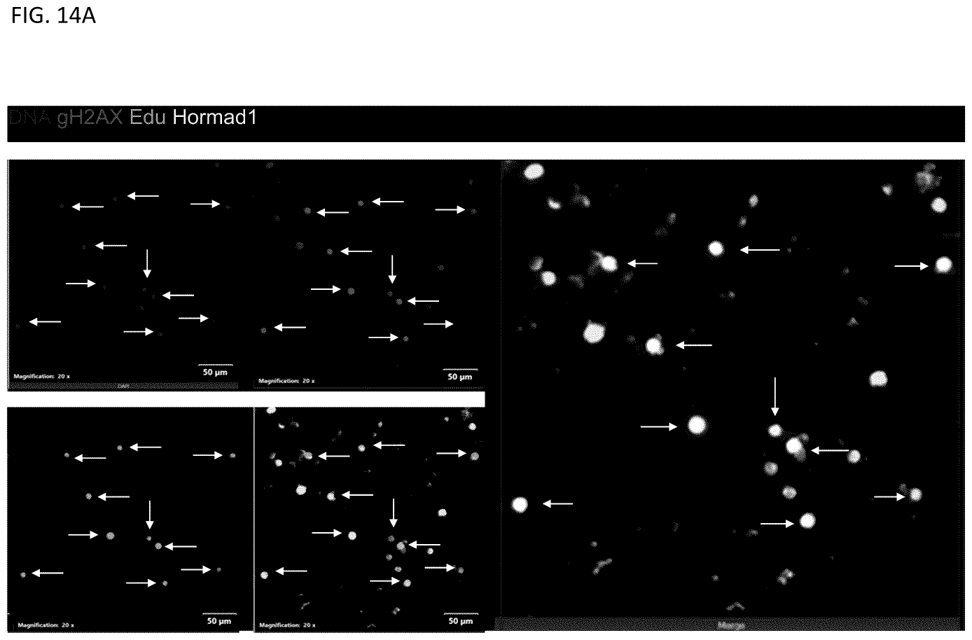

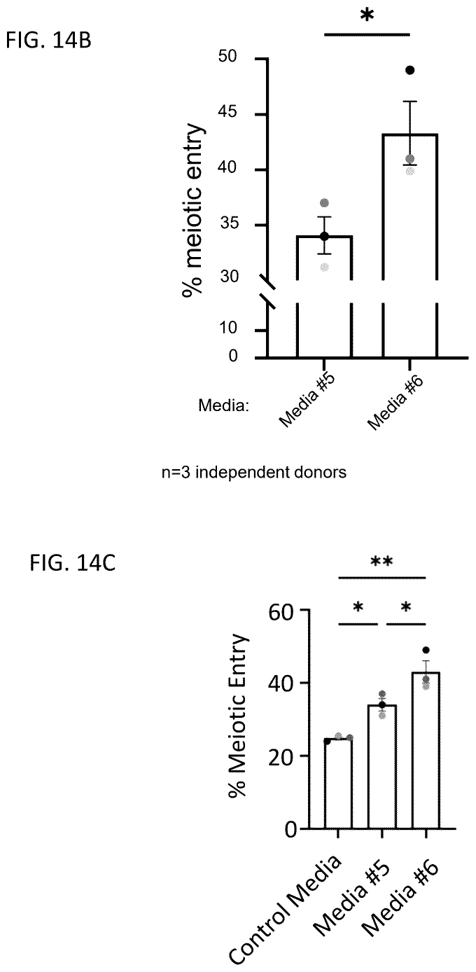

These optimized conditions resulted in a marked increase in meiotic entry efficiency, with the fraction of EdU-positive early meiotic cells rising to about 40% and up to approximately 60% (see FIGS. 14A to 14C). The highest efficiency, about 60% of cells entering early meiosis, was achieved when dSPGs were cultured for four days in medium #5 (Table 7) supplemented with insulin and IWR-endo-1 (referred to hereinafter as medium #6), although substantial meiotic entry was also observed with shorter culture periods in medium #6. This protocol consistently produced the highest proportion of cells expressing early meiotic markers, nearly triple the efficiency of medium #5 alone. Medium #6 is shown in Table 9.

TABLE 8

| | | | | | | | | Fraction |
|---|---|---|---|---|---|---|---|---|
| | | | | | | | | (%) |
| | | | | | | | | of |
| | | | | | Time | | | Edu + |
| | | | | | points | | | Early- |
| Media | | Concentrations | | Media | tested | Edu | Plate | stage |
| Foundation | Reagent | tested | | conditions | (days) | Concentration | modification | Meiosis |
| Alpha- | Insulin | 50 | ug/mL | 5 | 4 | 5 uM | NA | 24 |
| MEM | | | | | | | | |
| Alpha- | IGF-1 high | 1 | ug/mL | 5 | 4 | 5 uM | NA | 15 |
| MEM | | | | | | | | |
| Alpha- | 2DG | 1 | mM | 5 | 4 | 5 uM | NA | 24 |
| MEM | | | | | | | | |
| Alpha- | 2DG | 5 | mM | 5 | 4 | 5 uM | NA | 17 |
| MEM | | | | | | | | |
| Alpha- | Oxythiamine | 25 uM, | | 5 | 4 | 5 uM | NA | 26, 15 |
| MEM | | 100 uM | | | | | | |
| Alpha- | GSK | 5 | uM | 5 | 4 | 5 uM | NA | 18 |
| MEM | | | | | | | | |
| Alpha- | BAX inhibitor | 100 | uM | 5 | 4 | 5 uM | NA | 20 |
| MEM | peptide | | | | | | | |
| Alpha- | SNAP | 250 | uM | 5 | 4 | 5 uM | NA | 19 |
| MEM | | | | | | | | |
| Alpha- | BCL2 | 1 | ug/mL | 5 | 4 | 5 uM | NA | 22 |
| MEM | | | | | | | | |
| Alpha- | RA + AM580 | 5 uM + 5 uM | | 5 | 4 | 5 uM | NA | 19 |
| MEM | | | | | | | | |
| Alpha- | WNT1 | 200 | ng/mL | 5 | 4 | 5 uM | NA | 21 |
| MEM | | | | | | | | |
| Alpha- | WNT3A | 200 | ng/mL | 5 | 4 | 5 uM | NA | 19 |
| MEM | | | | | | | | |
| Alpha- | WNT-11 | 200 | ng/mL | 5 | 4 | 5 uM | NA | 17 |
| MEM | | | | | | | | |
| Alpha- | Laduviglusib | 3 | uM | 5 | 4 | 5 uM | NA | 19 |
| MEM | | | | | | | | |
| Alpha- | Laduviglusib | 10 | uM | 5 | 4 | 5 uM | NA | 19 |
| MEM | | | | | | | | |
| Alpha- | IWR-endo-1 | 3 | uM | 5 | 4 | 5 uM | NA | 29 |
| MEM | | | | | | | | |
| Alpha- | IWR-endo-1 | 10 | uM | 5 | 4 | 5 uM | NA | 20 |
| MEM | | | | | | | | |
| Alpha- | DKK1 | L280 20 uM | | 5 | 4 | 5 uM | NA | 24 |
| MEM | | | | | | | | |
| Alpha- | DKK2 | 200 | ng/mL | 5 | 4 | 5 uM | NA | 26 |
| MEM | | | | | | | | |
| Alpha- | EGF | 200 | ng/mL | 5 | 4 | 5 uM | NA | 21 |
| MEM | | | | | | | | |
| Alpha- | MDK | 100 | ng/ml | 5 | 4 | 5 uM | NA | 24 |
| MEM | | | | | | | | |
| Alpha- | CNTF | 100 | ng/ml | 5 | 4 | 5 uM | NA | 18 |
| MEM | | | | | | | | |
| Alpha- | NRG1 | 100 | ng/ml | 5 | 4 | 5 uM | NA | 16 |
| MEM | | | | | | | | |
| Alpha- | HGF | 100 | ng/ml | 5 | 4 | 5 uM | NA | 27 |
| MEM | | | | | | | | |
| Alpha- | WNT1, WNT3A, | 100 | ng/ml | 5 | 4 | 5 uM | NA | 18 |
| MEM | WNT-11, | | | | | | | |
| | Laduviglusib | | | | | | | |

| | | | | | | | |
|---|---|---|---|---|---|---|---|
| Alpha- | Laduviglusib + | 10 uM + | 5 | 4 | 5 uM | NA | 18 |
| MEM | IWR-endo-1 | 10 uM | | | | | |
| Alpha- | IGF-1, | 1 ug/mL, | 5 | 4 | 5 uM | NA | 23 |
| MEM | Laduviglusib Low | 3 uM | | | | | |
| Alpha- | IGF-1, IWR- | 1 ug/mL, | 5 | 4 | 5 uM | NA | 19 |
| MEM | endo-1 | 3 uM | | | | | |
| Alpha- | NA | NA | 5 | 4 | 5 uM | NA | 20 |
| MEM | | | | | | | |
| Alpha- | Insulin + IWR- | 50 ug/mL + | 5 | 4 | 5 uM | NA | 56 |
| MEM | endo-1 | 3 uM | | | | | |
| Alpha- | Insulin + IWR- | 50 ug/mL + | 5 (3 | 4 | 10 nM | NA | 41 |
| MEM | endo-1, Days | 3 uM | day), 2 | | | | |
| | 1, 2 and 3 | | (1 day) | | | | |
| Alpha- | Insulin + IWR- | 50 ug/mL + | 5 (2 | 4 | 10 nM | NA | 42 |
| MEM | endo-1, Days | 3 uM | day), 2 | | | | |
| | 1, 2 only | | (2 | | | | |
| | | | days) | | | | |
| Alpha- | Insulin + IWR- | 50 ug/mL + | 5 (1 | 4 | 10 nM | NA | 43 |
| MEM | endo-1, Day 1 | 3 uM | day), 2 | | | | |
| | only | | (3 | | | | |
| | | | days) | | | | |

TABLE 9

| Cell culture | | | | |
|---|---|---|---|---|
| media #6 | Concentration | | Vendor | Catalog |
| EBSS | 89% | | Gibco | 14155063 |
| Alpha-MEM | 10% | | Gibco | 12571048 |
| KSR | 1% | | Gibco | 10828028 |
| Pen/Strep | 1% | | Gibco | 15070063 |
| BMP2 | 20 | ng/mL | R&D Systems | 355-BEC-010/CF |
| BMP4 | 20 | ng/mL | R&D Systems | 314-BPE-050 |
| BMP7 | 20 | ng/mL | R&D Systems | 354-BP-10/CF |
| Retinoic Acid | 5 | uM | Sigma Aldrich | R2625-100MG |
| Activin A | 100 | ng/mL | R&D Systems | 338-GMP-010 |
| Insulin | 50 | ug/mL | | |
| IWR-endo-1 | 3 | uM | | |

However, it was also discovered that, although the cells entered meiosis and became primary spermatocytes, they consistently arrested at the early stages of meiotic prophase, specifically at the leptotene and zygotene stages, before reaching pachytene. This early meiotic arrest was marked by the expression of canonical markers such as HORMAD1 and γH2AX, indicating that homologous chromosome pairing and the onset of recombination had begun, but full synapsis and progression through later prophase I had not occurred.

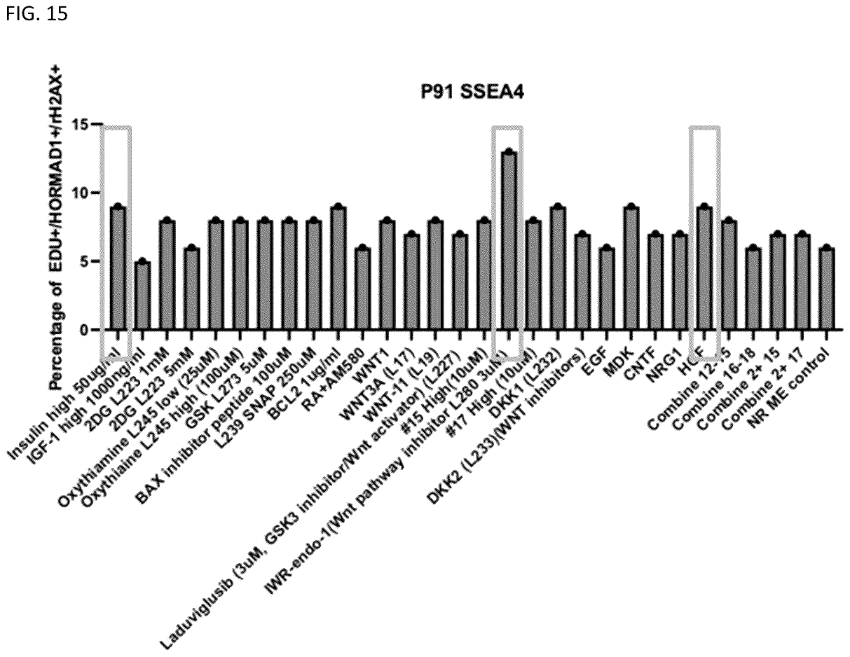

Similar experiments were also performed using isolated or cultured SSCs, and the inventors discovered that SSCs can also enter meiosis when cultured in culture conditions similar to those of dSPGs (FIG. 15).

### Example 4. Progression of Human Spermatocytes Through Meiosis to Meiotic Completion and Generation of Spermatids In Vitro

Recognizing the developmental block described in Example 3, new culture conditions were developed to promote further meiotic progression of human spermatocytes. The majority of conditions tested for meiotic progression, including a wide variety of base media, metabolic modulators, growth factors, and plate modifications such as Matrigel or Geltrex, did not result in successful progression of human primary spermatocytes beyond the early stages of meiotic prophase (Table 10). In these unsuccessful conditions, even with the addition of compounds such as okadaic acid, glucose, lactate, alpha-ketoglutarate, retinoic acid, VEGFB, IGF2, IL13, TGFB, BMP4, and others, the cells consistently failed to progress past the leptotene or zygotene stages to the pachytene stage, and no EdU+pachytene cells were observed.

TABLE 10

| | | | | | | | | | Observation |
|---|---|---|---|---|---|---|---|---|---|
| | | | | | Time | | | | of |
| | | | | | points | | | | Edu + |
| Media | | Concentrations | | Media | tested | | | Plate | Pachytene |
| Foundation | Reagent | tested | | conditions | (days) | Edu Con. | | modification | Cells? |
| Alpha- | NA | NA | | 1 | 1, 5, 10 | | | NA | No |
| MEM | | | | | | | | | |
| Alpha- | NA | NA | | 2 | 5, 11 | | | NA | No |
| MEM | | | | | | | | | |
| Alpha- | NA | NA | | 3 | 5, 11 | | | NA | No |
| MEM | | | | | | | | | |
| Alpha- | NA | NA | | 4 | 12 | | | NA | No |
| MEM | | | | | | | | | |
| Alpha- | Okadaic | 1 | uM | 1, 2 | 1, 5, 10 | 5 | uM | NA | No |
| MEM | acid | | | | | | | | |
| Alpha- | Glucose | 2 mM, 7 mM | | 2, 3 | 5, 11 | 5 | uM | NA | No |
| MEM | | | | | | | | | |
| Alpha- | Lactate | 0.1 mM, | | 2, 3 | 5, 11 | 5 | uM | NA | No |
| MEM | | 5 mM | | | | | | | |
| Alpha- | Alpha | 0.1 mM, | | 2, 3 | 5, 11 | 5 | uM | NA | No |
| MEM | Ketogluterate | 5 mM | | | | | | | |
| Alpha- | MDK | | | | | | | NA | No |
| MEM | | | | | | | | | |
| Alpha- | aMSH | 10 | nM | 2, 3 | 5, 11 | 5 | uM | NA | No |
| MEM | | | | | | | | | |
| Alpha- | Mel2 | 10 | nM | 2, 3 | 5, 11 | 5 | uM | NA | No |
| MEM | | | | | | | | | |
| Alpha- | Retinoic | 1 | uM | 1, 2 | | 5 | uM | NA | No |
| MEM | Acid | | | | | | | | |
| Alpha- | VEGFB | 100 ng/mL, | | 2, 4 | 7, 12 | 10 | nM | NA | No |
| MEM | | 10 ng/mL | | | | | | | |
| Alpha- | PGF | | | | | 10 nM, | | NA | No |
| MEM | | | | | | 50 nM | | | |
| Alpha- | IGF2 | 10 | ng/mL | 2, 4 | 7, 12 | 10 nM, | | NA | No |
| MEM | | | | | | 50 nM | | | |
| Alpha- | IL13 | 100 | ng/mL | 1, 2 | 1, 5, 10 | 5 | uM | NA | No |
| MEM | | | | | | | | | |
| Alpha- | GRN | 10 | ng/mL | 2, 4 | 7 | 50 | nM | NA | No |
| MEM | | | | | | | | | |
| Alpha- | TGFb | 10 | ng/mL | 2, 4 | 7 | 10 nM, | | NA | No |
| MEM | | | | | | 50 nM | | | |
| Alpha- | BMP4 | 100 ng/mL, | | 2, 4 | 7 | 10 | nM | NA | No |
| MEM | | 10 ng/mL | | | | | | | |
| Alpha- | YC-1 | 15 | uM | 2 | 12 | 10 | nM | NA | No |
| MEM | | | | | | | | | |
| Alpha- | CoCl2 | 150 | uM | 2 | 12 | 10 | nM | NA | No |
| MEM | | | | | | | | | |
| Alpha- | Insulin | 50 | ug/mL | 2 | 0, 10 | 0, 10 | nM | NA | No |
| MEM | | | | | | | | | |
| Alpha- | HGF | 1 | ug/mL | 2 | 0, 10 | 0, 10 | nM | NA | No |
| MEM | | | | | | | | | |
| Alpha- | NA | NA | | 2 | 0, 10 | 0, 10 | | NA | No |
| MEM | | | | | | | | | |
| Alpha- | NA | NA | | 2 | 4, 8 | 0, 50, | | Matrigel, | No |
| MEM | | | | | | 500 nM | | Geltrex | |
| APEL2 | NA | NA | | 2 | 4, 8 | 0, 50, | | Matrigel, | No |
| | | | | | | 500 nM | | Geltrex | |
| Alpha- | NA | NA | | 2 | 1, 5, 10 | 0 | | Matrigel | No |
| MEM | | | | | | | | | |
| Alpha- | Insulin + | 50 ug/mL + | | 2 + 5 | 1, 5, 10 | 0 | | Matrigel | No |
| MEM | HGF | 200 ng/mL | | | | | | | |
| DMEM/F12 | NA | NA | | 2 | 1, 5, 10 | 0 | | Matrigel | No |
| DMEM/F12 | Insulin + | 50 ug/mL + | | 2 + 5 | 1, 5, 10 | 0 | | Matrigel | No |
| | HGF | 200 ng/mL | | | | | | | |
| APEL2 | NA | NA | | 2 | 1, 5, 10 | 0 | | Matrigel | No |
| APEL2 | Insulin + | 50 ug/mL + | | 2 + 5 | 1, 5, 10 | 0 | | Matrigel | No |
| | HGF | 200 ng/mL | | | | | | | |
| Alpha- | NA | NA | | 1, 2 | 8 | 5 | uM | Matrigel | No |
| MEM | | | | | | | | | |
| DMEM/F12 | NA | NA | | 1, 2 | 8 | 5 | uM | Matrigel | No |
| APEL2 | NA | NA | | 1, 2 | 8 | 5 | uM | Matrigel | No |
| APEL2 | HGF | 1 | ug/mL | 2 | 8 | 5 | uM | Matrigel | No |
| APEL2 | Retinoic | 5 | uM | 2 | 8 | 5 | uM | Matrigel | No |
| | Acid | | | | | | | | |
| Alpha- | 3BP | 100 uM, | | 2 | 8 | 5 | uM | Matrigel | No |
| MEM | | 500 uM | | | | | | | |
| Alpha- | 2DG | 1 mM, 5 mM | | 2 | 8 | 5 | uM | Matrigel | No |
| MEM | | | | | | | | | |
| Alpha- | Oxythiamine | 100 uM, | | 2 | 8 | 5 | uM | Matrigel | No |
| MEM | | 500 uM | | | | | | | |
| Alpha- | GSK3484862 + | 5 uM + 5 uM | | 1 | 5, 11 | 5 | uM | NA | No |
| MEM | Retinoic | | | | | | | | |
| | Acid | | | | | | | | |
| APEL2 | GSK3484862 + | 5 uM + 5 uM | | 1 | 5, 11 | 5 | uM | NA | No |
| | Retinoic | | | | | | | | |
| | Acid | | | | | | | | |
| Alpha- | Insulin + | 50 ug/mL + | | 5 | 5, 11 | 5 | uM | NA | No |
| MEM | BCL2 | 1 ug/mL | | | | | | | |
| APEL2 | Insulin + | 50 ug/mL + | | 5 | 5, 11 | 5 | uM | NA | No |
| | BCL2 | 1 ug/mL | | | | | | | |
| Alpha- | Insulin + | 50 ug/mL + | | 5 | 5, 11 | 5 | uM | Matrigel | No |
| MEM | BCL2 | 1 ug/mL | | | | | | | |
| APEL2 | Insulin + | 50 ug/mL + | | 5 | 5, 11 | 5 | uM | Matrigel | No |
| | BCL2 | 1 ug/mL | | | | | | | |
| Alpha- | Retinoic | 5 uM + 25 uM | | 1 | 5, 11 | 5 | uM | NA | No |
| MEM | Acid + | | | | | | | | |
| | DHC | | | | | | | | |
| Alpha- | Retinoic | 5 uM + 25 uM | | 1 | 5, 11 | 5 | uM | NA | No |
| MEM | Acid + | | | | | | | | |
| | DHC | | | | | | | | |
| APEL2 | Retinoic | 5 uM + 25 uM | | 1 | 5, 11 | 5 | uM | NA | No |
| | Acid + | | | | | | | | |
| | DHC | | | | | | | | |
| APEL2 | Retinoic | 5 uM + 25 uM | | 1 | 5, 11 | 5 | uM | NA | No |
| | Acid + | | | | | | | | |
| | DHC | | | | | | | | |
| M199 | NA | NA | | 2 | 5, 8 | 5 | uM | NA | No |
| Alpha- | ZnCl2 | 1 | nM | 2 | 5, 8 | 5 | uM | NA | No |
| MEM | | | | | | | | | |

| | | | | | | | | |
|---|---|---|---|---|---|---|---|---|
| Alpha- | ZnCl2 + | 0.5 nM + | 2 | 5, 8 | 5 | uM | NA | No |
| MEM | BLC2 | 2 ug/mL | | | | | | |
| Alpha- | NA | NA | 2 | 5, 10 | 0, 10 | nM | Mouse | Yes |
| MEM | | | | | | | Embryonic | |
| | | | | | | | Fibroblasts | |
| APEL2 | NA | NA | 2 | 5, 10 | 0, 10 | nM | Mouse | Yes |
| | | | | | | | Embryonic | |
| | | | | | | | Fibroblasts | |
| Alpha- | Insulin, | 50 ug/mL + | 2 | 5, 10 | 0, 10 | nM | Mouse | Yes |
| MEM | HGF, DHT | 1 ug/mL + | | | | | Embryonic | |
| | | 1 uM | | | | | Fibroblasts | |

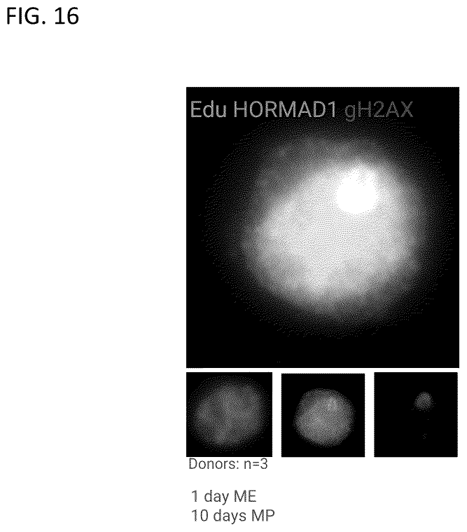

Of all the tested conditions, only the last three conditions in Table 10 were successful in supporting meiotic progression to the pachytene stage (FIG. 16) and beyond. In all three of these successful conditions, the use of media #2, with αMEM or APEL2 as the media foundation, and culturing on mouse embryonic fibroblast (MEF) feeder layers, were both essential. Media #2 is a nutrient-rich medium characterized by αMEM or APEL2 supplemented with KSR, FSH, a high concentration of testosterone, and bovine pituitary extract (Table 4), providing a supportive environment for meiotic progression. The use of other supports such as Matrigel was not successful. Additionally, the medium could include additional factors such as insulin, HGF, and DHT, or use APEL2 as the media foundation, but these were not essential for progression. Importantly, none of the three successful conditions included the other reagents that were tested in combination with the nutrient-rich media elsewhere in the data set, such as metabolic modulators or additional growth factors, which were found to be incompatible with meiotic progression in this context.

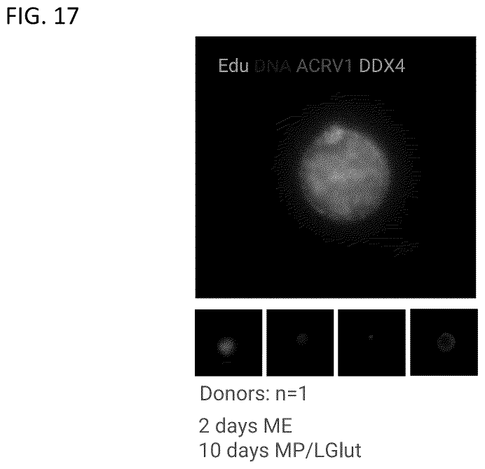

It was further discovered that supplementing media #2 with L-glutamine at varying concentrations (1 mM, 2 mM, 3 mM, or 4 mM) or with Glutamax (4 mM) for 5 to 10 days was capable of supporting not only meiotic progression but also completion of meiosis and the formation of haploid spermatids (FIG. 17). The addition of an agonist of a c-KIT receptor, such as KIT ligand (KITLG), further improved the efficiency of meiotic progression and completion. In all cases, cultures were performed on MEF feeder layers, which were found to be essential for both meiotic progression and completion. In contrast, when other media or additional reagents were used in place of media #2, or when MEF feeder cells were omitted, no progression to haploid spermatids was observed. This underscores the importance of the supportive role of MEF feeder cells and the use of L-glutamine or Glutamax in enabling the completion of meiosis and the formation of haploid spermatids from human germ cells in vitro. Media #2 supplemented with L-glutamine or Glutamax is referred to hereinafter as Media #7 (Table 11), and media #2 supplemented with L-glutamine or Glutamax and KITLG is referred to as Media #8 (Table 12).

No further differentiation was observed after the formation of round spermatids described in this example.

TABLE 11

| Cell culture media #7 | Concentration | | Vendor | Catalog |
|---|---|---|---|---|
| αMEM, APEL2, | 89% | | Gibco | 12571048 |
| M199, or DMEM/F12 | | | | |
| Knockout Serum | 10% | | Gibco | 10828028 |
| Replacement (KSR) | | | | |
| Pen/Strep | 1% | | Gibco | 15070063 |
| Follicle Stimulating | 20 | ng/mL | Sigma | F4021-10UG |
| Hormone (FSH) | | | Aldrich | |
| Testosterone 10 uM | 10 | uM | | |
| Bovine Pituitary | 50 | ug/mL | Corning | 354123 |
| Extract (BPE) | | | | |

| | |
|---|---|
| L-Glutamine | 1 mM, 2 mM, 3 mM, |
| or Glutamax | 4 mM L-Glutamine; or |
| | 4 mM Glutamax |

TABLE 12

| Cell culture media #8 | Concentration | | Vendor | Catalog |
|---|---|---|---|---|
| αMEM, APEL2, | 89% | | Gibco | 12571048 |
| M199, or DMEM/F12 | | | | |
| Knockout Serum | 10% | | Gibco | 10828028 |
| Replacement (KSR) | | | | |
| Pen/Strep | 1% | | Gibco | 15070063 |
| Follicle Stimulating | 20 | ng/mL | Sigma | F4021-10UG |
| Hormone (FSH) | | | Aldrich | |
| Testosterone 10 uM | 10 | uM | | |
| Bovine Pituitary | 50 | ug/mL | Corning | 354123 |
| Extract (BPE) | | | | |

| | |
|---|---|
| L-Glutamine | 1 mM, 2 mM, 3 mM, |
| or Glutamax | 4 mM L-Glutamine; or |
| | 4 mM Glutamax |
| KITLG | 1 ng/mL |

### Example 5. Spermiogenesis from Human Round Spermatids In Vitro

As explained herein above in Example 4, no further differentiation was observed after the formation of round spermatids. Accordingly, a series of experiments were conducted to evaluate conditions for supporting successful spermiogenesis in vitro using round spermatids. The round spermatids used in these experiments were obtained either directly from human testicular tissue or are generated in vitro as described in Example 4. While not all tested conditions are shown, the inventors systematically explored a variety of media formulations and culture environments.

After extensive experimentation following the methods described in Examples 2-4, the inventors discovered that a nutrient-rich medium, designated as SP1 medium or media #9 (Table 13), comprising the αMEM, Knockout Serum Replacement (KSR), penicillin-streptomycin of base media #1, supplemented with 2-mercaptoethanol, a high concentration of testosterone, and retinoic acid, was able to support the generation of a small proportion, about 4.5%, of elongated spermatids from round spermatids when cultured for 3-6 days.

TABLE 13

| Cell culture media #9 | Concentration | Vendor | Catalog |
|---|---|---|---|
| αMEM, APEL2, M199, | 89% | Gibco | 12571048 |
| or DMEM/F12 | | | |
| Knockout Serum | 10% | Gibco | 10828028 |
| Replacement (KSR) | | | |
| Pen/Strep | 1% | Gibco | 15070063 |
| Follicle Stimulating | 20 ng/mL | Sigma | F4021-10UG |
| Hormone (FSH) | | Aldrich | |
| 2-mercaptoethanol | | | |
| testosterone | | | |
| Retinoic acid | | | |

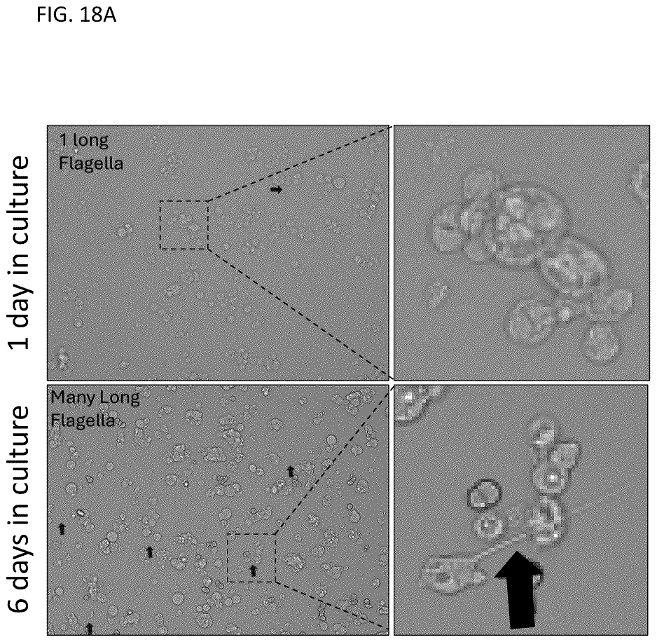

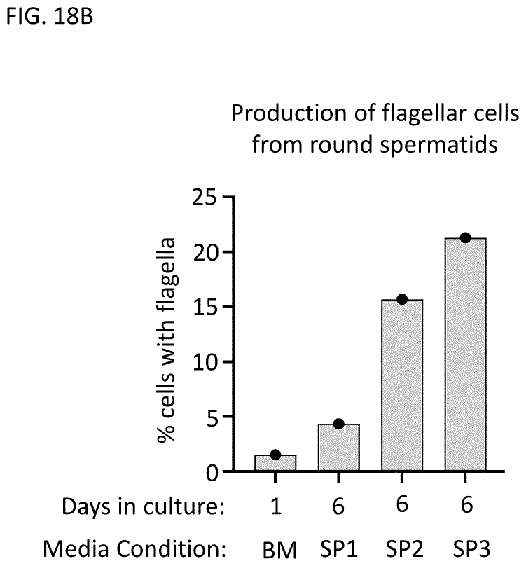

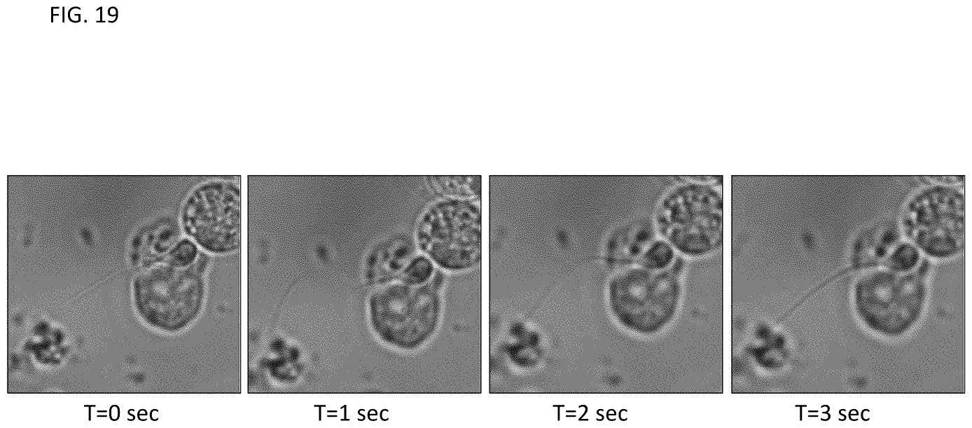

Further optimization revealed that the addition of lactate to the SP medium (referred to hereinafter as SP2 or media #10, Table 14) increased the efficiency of spermiogenesis to 15.7% of elongated spermatids, flagellated spermatids, the spermatozoa (FIG. 19). In another set of experiments, SP1 was supplemented with SP1 medium conditioned with mouse embryonic fibroblast (MEF) cells. SP1 was conditioned by plating 200,000 MEFs in base medium in a 12-well plate. The following day, the medium was changed to SP1 (without retinoic acid). On the day of plating or media change, conditioned medium was harvested from the MEFs, centrifuged at 17,000×g for 10 minutes, diluted 1:3 in SP1, and then retinoic acid was added. When round spermatids were cultured in SP1 medium comprising MEF-conditioned SP1 diluted at 1:3, up to 21% of the round spermatids successfully differentiated into elongated spermatids after 3-6 days of culture (see FIGS. 18A and 18B) and then spermatozoa (FIG. 19). MEF-conditioned SP1 medium is referred to hereinafter as SP3 or media #11 and is shown in Table 15.

TABLE 14

| Cell culture | | | | |
|---|---|---|---|---|
| media #10 | Concentration | | Vendor | Catalog |
| αMEM, APEL2, M199, | 89% | | Gibco | 12571048 |
| or DMEM/F12 | | | | |
| Knockout Serum | 10% | | Gibco | 10828028 |
| Replacement (KSR) | | | | |
| Pen/Strep | 1% | | Gibco | 15070063 |
| Follicle Stimulating | 20 | ng/mL | Sigma | F4021- |
| Hormone (FSH) | | | Aldrich | 10UG |
| 2-mercaptoethanol | 50 | uM | | |
| testosterone | 5 | uM | | |
| Retinoic acid | 5 | uM | | |
| Lactate | 1 | mM | | |

TABLE 15

| Cell culture media #11 | Concentration | | Vendor | Catalog |
|---|---|---|---|---|
| αMEM, APEL2, M199, | 89% | | Gibco | 12571048 |
| or DMEM/F12 | | | | |
| Knockout Serum | 10% | | Gibco | 10828028 |
| Replacement (KSR) | | | | |
| Pen/Strep | 1% | | Gibco | 15070063 |
| Follicle Stimulating | 20 | ng/mL | Sigma | F4021- |
| Hormone (FSH) | | | Aldrich | 10UG |
| 2-mercaptoethanol | 50 | uM | | |
| testosterone | 5 | uM | | |
| Retinoic acid | 5 | uM | | |

| | |
|---|---|
| MEF-conditioned media | 1:3 in SP1 |
| | by volume |

To evaluate the motility of the spermatozoa generated in vitro, the inventors conducted experiments using Pentoxyfiline, a methylxanthine derivative commonly employed in reproductive biology to stimulate and assess sperm motility. Pentoxyfiline acts as a phosphodiesterase inhibitor, increasing intracellular cyclic AMP levels and thereby enhancing sperm movement and hyperactivation. It is frequently used in clinical and research settings to assess the functional capacity of spermatozoa, particularly in the context of assisted reproductive technologies.

In this experiment, spermatozoa produced from the in vitro differentiation protocol were exposed to Pentoxyfiline at concentrations of 0.5 mg/ml and 1.5 mg/mL. No pre-incubation was performed; Pentoxyfiline was added directly to the culture, and the cells were observed for motility within minutes of exposure. At a concentration of 0.5 mg/mL, an increase in sperm movement was observed, indicating activation of motility. At the higher concentration of 1.5 mg/mL, pronounced and vigorous tail movement was seen, with some spermatozoa exhibiting rapid, hyperactivated motility.

These results demonstrate that the in vitro-generated spermatozoa are responsive to Pentoxyfiline stimulation and are capable of exhibiting motility, further supporting the functional maturation of the cells produced by the disclosed culture system.

These results demonstrate that both the composition of the culture medium and the use of conditioned media can significantly enhance the efficiency of in vitro spermiogenesis from round spermatids.

### Example 6. Complete In Vitro Differentiation of Human SSCs or dSPGs to Spermatozoa

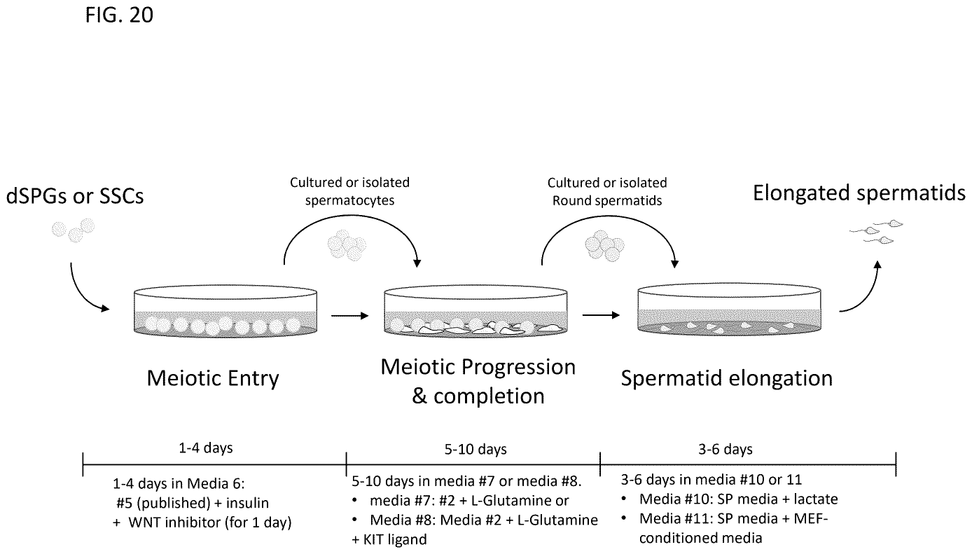

Building on the results described in Examples 2 through 5, the inventors combined the optimized, stage-specific culture conditions into a sequential protocol to achieve the complete in vitro differentiation of human differentiating spermatogonia (dSPGs) to spermatozoa (FIG. 20). Each step in this process was based on the most effective conditions identified for meiotic entry, meiotic progression, meiotic completion, and spermiogenesis.

In this integrated approach, dSPGs were first cultured in a nutrient-restricted meiotic entry medium supplemented with morphogens and pathway modulators, including BMP2, BMP4, BMP7, retinoic acid, activin A, insulin, and a WNT pathway inhibitor, without the use of feeder cells. This step induced efficient entry into meiosis, as evidenced by the appearance of Edu+, HORMAD1+, and γH2AX+ primary spermatocytes.

Following meiotic entry, the cells were transferred to a nutrient-rich progression medium containing αMEM, KSR, FSH, testosterone, and bovine pituitary extract, with the addition of mouse embryonic fibroblast (MEF) feeder cells. This environment enabled progression through the pachytene stage of meiotic prophase I and completion of meiosis, resulting in the formation of haploid round spermatids, as confirmed by the loss of pre-pachytene markers and the appearance of spermatid-specific markers such as ACRV1.

For the final stage, round spermatids were cultured in a nutrient-rich spermiogenesis medium (SP medium) containing αMEM, KSR, penicillin-streptomycin, 2-mercaptoethanol, a high concentration of testosterone, retinoic acid, and, in some conditions, lactate or MEF-conditioned medium. Under these optimized conditions, a significant proportion of round spermatids differentiated into elongated, spermatozoa over 3-6 days, as visualized by immunofluorescence and phase-contrast microscopy.

Importantly, the inventors discovered that culturing cells at 32° C. throughout the culturing process significantly improved spermatogenesis.

What is claimed is:

1. A method of generating human spermatozoa from differentiating spermatogonia (dSPGs) or spermatogonial stem cells (SSCs) in vitro, the method comprising:
a. culturing human dSPGs or SSCs in a first culture medium of a first culture system at a temperature ranging from about 31° C. to about 33° C. for a first culture period sufficient for entry of the human dSPGs or SSCs into meiosis, wherein the first culture medium comprises:
i. a nutrient-rich basal medium and a serum or serum substitute, wherein the nutrient-rich basal medium is present at a sufficiently low concentration to provide a nutrient-restricted medium, wherein the sufficiently low concentration of nutrient-rich basal medium ranges from about 8% to about 12% of the total medium volume and the balance comprising a diluent or buffered salt solution at a concentration ranging from about 88% to about 92%;
ii. retinoic acid at a concentration ranging from about 4 uM to about 6 uM;
iii. BMP2, BMP4 and BMP7 each at a concentration ranging from about 15 ng/ml to about 25 ng/ml;
iv. activin A at a concentration ranging from about 90 ng/ml to about 110 ng/ml;
V. insulin at a concentration ranging from about 40 ug/mL to about 60 ug/mL; and
vi. IWR-endo-1 at a concentration ranging from about 2 uM to about 4 uM; and
b. transferring the cells resulting from culturing in the first culture medium to a second culture medium of a second culture system and culturing the cells using the second culture system at a temperature ranging from about 31° C. to about 33° C. for a second culture period sufficient for the progression of the human spermatocytes arrested before the pachytene stage of meiotic prophase I through completion of meiosis and formation of round spermatids, the second culture system comprising:
i. a second culture medium comprising
1) a nutrient-rich basal medium and a serum or serum substitute, wherein the nutrient-rich basal medium is present at a sufficiently high concentration to provide a nutrient-rich medium, wherein the sufficiently high concentration of nutrient-rich basal medium ranges from about 85% to about 95% of the total medium volume;
2) FSH at a concentration ranging from about 15 ng/ml to about 25 ng/ml;
3) testosterone at a concentration ranging from about 9 uM to about 11 uM;
4) bovine pituitary extract (BPE) at a concentration ranging from about 40 ug/mL to about 60 ug/mL;
5) L-Glutamine or a L-alanyl-L-glutamine dipeptide at a concentration ranging from about 0.5 mM to about 5 mM; and
6) optionally c-KIT receptor ligand (KITLG) at a concentration ranging from about 0.5 ng/ml to about 1.5 ng/ml; and
ii. MEF feeder cells; and
c. transferring the cells resulting from culturing in the second culture system to a third culture medium of a third culture system and culturing the cells in the third culture system at a temperature ranging from about 31° C. to about 33° C. for a third culture period sufficient for generation of human spermatozoa from the round spermatids, wherein the third culture medium comprises:
i. a nutrient-rich basal medium and a serum or serum substitute, wherein the nutrient-rich basal medium is present at a sufficiently high concentration to provide a nutrient-rich medium, wherein the sufficiently high concentration of nutrient-rich basal medium ranges from about 85% to about 95% of the total medium volume;
ii. 2-mercaptoethanol at a concentration ranging from about 45 uM to about 55 uM;
iii. testosterone at a concentration ranging from about 3 uM to about 7 uM
iv. retinoic acid at a concentration ranging from about 4 uM to about 6 uM; and
v. conditioned media comprising MEF-conditioned third culture medium, lactate at a concentration ranging from about 0.8 mM to about 1.2 mM, or a combination thereof;
wherein entry of the human dSPGs and SSCs into meiosis is evidenced by EdU incorporation indicating DNA replication and the appearance of spermatocytes arrested before the pachytene stage of meiosis I as characterized by the presence, absence, or localization of HORMAD1, γH2AX, DMC1, RAD51, STRA8, or any combination thereof; wherein progression of the human spermatocytes arrested before the pachytene stage of meiotic prophase I through completion of meiosis and formation of round spermatids is evidenced b by the appearance of cells expressing ACRV1; and wherein generation of human spermatozoa from the round spermatids is evidenced by the appearance of flagellar cells.

2. The method of claim 1, wherein the first culture medium comprises αMEM basal medium at a concentration ranging from about 8% to about 12%.

3. The method of claim 1, wherein the first culture medium comprises KSR serum substitute at a concentration ranging from about 0.1% to about 2%.

4. The method of claim 1, wherein the first culture medium comprises αMEM basal medium at a concentration ranging from about 8% to about 12%, EBSS at a concentration ranging from about 88% to about 92%, KSR serum substitute at a concentration ranging from about 0.1% to about 2%, BMP2, BMP4, and BMP7 each at a concentration ranging from about 15 ng/ml to about 25 ng/ml, retinoic acid at a concentration ranging from about 4 uM to about 6 uM, activin A at a concentration ranging from about 90 ng/ml to about 110 ng/ml, insulin at a concentration ranging from about 40 ug/mL to about 60 ug/mL, and IWR-endo-1 at a concentration ranging from about 2 uM to about 4 uM.

5. The method of claim 1, wherein entry of the human dSPGs and SSCs into meiosis is further evidenced by the appearance of spermatocytes arrested before the pachytene stage of meiosis I characterized by EdU incorporation indicating DNA replication; the presence of HORMAD1 on unsynapsed chromosome axes and presence of γH2AX diffusely throughout the nucleus, prior to its restriction to the sex body; absence, reduction, or relocalization of HORMAD1 and γH2AX from autosomes; or any combination thereof.

6. The method of claim 1, wherein the first culture period is about 1 day to about 4 days.

7. The method of claim 1, wherein the second culture medium comprises αMEM at a concentration ranging from about 85% to about 95%.

8. The method of claim 1, wherein the second culture medium comprises KSR at a concentration ranging from about 8% to about 12%.

9. The method of claim 1, wherein the second culture period ranges from about 8 days to about 12 days.

10. The method of claim 1, wherein the third culture medium comprises MEF-conditioned third culture medium at a rate of one part MEF-conditioned third cell culture medium to three parts third culture medium.

11. The method of claim 1, wherein the third culture period is about 5 to about 8 days.
